SEQUENCE LISTING (108 sequences)

SEQ ID NO: 1 - RNA, 21 nt, artificial
  sense strand of siRNA targeted to AR
caagggagguuacaccaaauu

SEQ ID NO: 2 - RNA, 21 nt, artificial
  antisense strand of siRNA targeted to AR
uuugguguaaccucccuuguu

SEQ ID NO: 3 - RNA, 21 nt, artificial
  sense strand of siRNA targeted to AR
ggaacucgaucguaucauuuu

SEQ ID NO: 4 - RNA, 21 nt, artificial
  antisense strand of siRNA targeted to AR
aaugauacgaucgaguuccuu

SEQ ID NO: 5 - DNA, 42 bp, artificial
  synthetic shRNA targeted to AR
gatccccgccatactgcatggcagcattcaagagatgctgcc

SEQ ID NO: 6 - DNA, 64 bp, artificial
  synthetic shRNA targeted to AR
agcttttccaaaaagccatactgcatggcagcatctcttgaatgctgccatgcagtatggcggg

SEQ ID NO: 7 - DNA, 64 bp, artificial
  synthetic shRNA targeted to AR
gatccccggaactcgatcgtatcattttcaagagaaatgatacgatcgagttcctttttggaaa

SEQ ID NO: 8 - DNA, 64 bp, artificial
  synthetic shRNA targeted to AR
agcttttccaaaaaggaactcgatcgtatcatttctcttgaaaatgatacgatcgagttccggg

SEQ ID NO: 9 - DNA, 20 bp, artificial
  synthetic oligonucleotide primer
aacagaagtacctgtgcgcc

SEQ ID NO: 10 - DNA, 20 bp, artificial
  synthetic oligonucleotide primer
tgagactccaaacaccctca

SEQ ID NO: 11 - DNA, 20 bp, artificial
  synthetic oligonucleotide primer
tatgacactctgctgcctgc

SEQ ID NO: 12 - DNA, 20 bp, artificial
  synthetic oligonucleotide primer
tcagggtctggtcattttga

SEQ ID NO: 13 - DNA, 20 bp, artificial
  synthetic oligonucleotide primer
ttctgtcagtcccattggtg

SEQ ID NO: 14 - DNA, 20 bp, artificial
  synthetic oligonucleotide primer
tggatggatagctactccgg

SEQ ID NO: 15 - DNA, 24 bp, artificial
  synthetic oligonucleotide primer
gttcattctgaaaaatccttcagc

SEQ ID NO: 16 - DNA, 20 bp, artificial
  synthetic oligonucleotide primer
tggatggatagctactccgg

SEQ ID NO: 17 - DNA, 20 bp, artificial
  synthetic oligonucleotide primer
cccagaagcttcatctccac

SEQ ID NO: 18 - DNA, 24 bp, artificial
  synthetic oligonucleotide primer
ttcagattaccaagtttcttcagc

SEQ ID NO: 19 - DNA, 7756 bp, artificial
  synthetic vector encoding shRNA-resistant, wild-type AR
cccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgcgcgtatcgataaagcttgcatgcctgcaggtcgactctagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatggcccaaccggtgggacatttgagttgcttgcttggcactgtcctctcatgcgttgggtccactcagtagatgcctgttgaattgggtacgcggccagcttctgtggaatgtgtgtcagttagggtgtggaaagtccccaggctccccagcaggcagaagtatgcaaagcatgcatctcaattagtcagcaaccaggtgtggaaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaattccctatagtgagtcgtattaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttc

SEQ ID NO: 20 - DNA, 24 bp, artificial
  synthetic oligonucleotide primer
tgggatatccagccaagctcaagg

SEQ ID NO: 21 - DNA, 22 bp, artificial
  synthetic oligonucleotide primer
gggagtcgacacagggatgcca

SEQ ID NO: 22 - DNA, 38 bp, artificial
  synthetic oligonucleotide primer
ggaagctgcaaggtcttctagaaaagagccgctgaagg

SEQ ID NO: 23 - DNA, 38 bp, artificial
  synthetic oligonucleotide primer
ccttcagcggctcttttctagaagaccttgcagcttcc

SEQ ID NO: 24 - DNA, 56 bp, artificial
  synthetic oligonucleotide primer
ctagaaaagagccgctgaaggatttttcagaatgaacaaattaaaagaatcataag

SEQ ID NO: 25 - DNA, 56 bp, artificial
  synthetic oligonucleotide primer
ctagcttatgattcttttaatttgttcattctgaaaaatccttcagcggctctttt

SEQ ID NO: 26 - DNA, 36 bp, artificial
  synthetic oligonucleotide primer
gaagcagggatgactctagaagcccggaagctgaag

SEQ ID NO: 27 - DNA, 36 bp, artificial
  synthetic oligonucleotide primer
cttcagcttccgggcttctagagtcatccctgcttc

SEQ ID NO: 28 - DNA, 43 bp, artificial
  synthetic oligonucleotide primer
ctaggaggatttttcagaatgaacaaattaaaagaatcataat

SEQ ID NO: 29 - DNA, 43 bp, artificial
  synthetic oligonucleotide primer
ctagattatgattcttttaatttgttcattctgaaaaatcctc

SEQ ID NO: 30 - DNA, 67 bp, artificial
  synthetic oligonucleotide primer
ctaggagctgttgttgtttctgaaagaatcttgagggtgtttggagtctcagaatggcttccttaat

SEQ ID NO: 31 - DNA, 67 bp, artificial
  synthetic oligonucleotide primer
ctagattaaggaagccattctgagactccaaacaccctcaagattctttcagaaacaacaacagctc

SEQ ID NO: 32 - DNA, 28 bp, artificial
  synthetic oligonucleotide primer
ctaggagcaggcagcagagtgtcataat

SEQ ID NO: 33 - DNA, 28 bp, artificial
  synthetic oligonucleotide primer
ctagattatgacactctgctgcctgctc

SEQ ID NO: 34 - DNA, 58 bp, artificial
  synthetic oligonucleotide primer
ctaggagaaaaattccgggttggcaattgcaagcatctcaaaatgaccagaccctgat

SEQ ID NO: 35 - DNA, 58 bp, artificial
  synthetic oligonucleotide primer
ctagatcagggtctggtcattttgagatgcttgcaattgccaacccggaatttttctc

SEQ ID NO: 36 - DNA, 15 bp, homo sapiens
  misc_feature (7)..(9): n is a, c, g, or t
agaacannnagaaca

SEQ ID NO: 37 - DNA, 15 bp, homo sapiens
  misc_feature (7)..(9): n is a, c, g, or t
tgttctnnntgttct

SEQ ID NO: 38 - protein, 15 aa, homo sapiens
MTLGGFFRMNKLKES

SEQ ID NO: 39 - protein, 24 aa, homo sapiens
MTLGAAVVVSERILRVFGVSEWLP

SEQ ID NO: 40 - protein, 10 aa, homo sapiens
MTLGAGSRVS

SEQ ID NO: 41 - protein, 20 aa, homo sapiens
MTLGEKFRVGNCKHLKMTRP

SEQ ID NO: 42 - protein, 28 aa, homo sapiens
MTLGARKLKKLGNLKLQEEGEASSTTSP

SEQ ID NO: 43 - DNA, 80 bp, homo sapiens
catttgtcaattttggcttttgttggtacattagccatttgttggtaatggtagtgtacattacccatatattatatata

SEQ ID NO: 44 - DNA, 79 bp, homo sapiens
catttgtcaattttggcttttgttgccattgcttttggtgttttagacatgagtccttgccggtgcctatgtcatgaag

SEQ ID NO: 45 - DNA, 81 bp, homo sapiens
catttgtccaattttggcttttgttggtacattagccatttgttggtaatggtagtgtacattacccatatattatatata

SEQ ID NO: 46 - DNA, 80 bp, homo sapiens
gtgagttctttagtagtggattttgagattttagtgcagcagccacctgagaagtgtacattacccatatattatatata

SEQ ID NO: 47 - DNA, 20 bp, artificial
  synthetic oligonucleotide primer
aacagaagtacctgtgcgcc

SEQ ID NO: 48 - DNA, 20 bp, artificial
  synthetic oligonucleotide primer
tgagactccaaacaccctca

SEQ ID NO: 49 - DNA, 20 bp, artificial
  synthetic oligonucleotide primer
tatgacactctgctgcctgc

SEQ ID NO: 50 - DNA, 20 bp, artificial
  synthetic oligonucleotide primer
tcagggtctggtcattttga

SEQ ID NO: 51 - DNA, 20 bp, artificial
  synthetic oligonucleotide primer
ttctgtcagtcccattggtg

SEQ ID NO: 52 - DNA, 20 bp, artificial
  synthetic oligonucleotide primer
tggatggatagctactccgg

SEQ ID NO: 53 - DNA, 24 bp, artificial
  synthetic oligonucleotide primer
gttcattctgaaaaatccttcagc

SEQ ID NO: 54 - DNA, 20 bp, artificial
  synthetic oligonucleotide primer
tggatggatagctactccgg

SEQ ID NO: 55 - DNA, 20 bp, artificial
  synthetic oligonucleotide primer
cccagaagcttcatctccac

SEQ ID NO: 56 - DNA, 24 bp, artificial
  synthetic oligonucleotide primer
ttcagattaccaagtttcttcagc

SEQ ID NO: 57 - DNA, 20 bp, artificial
  synthetic oligonucleotide primer
tggatggatagctactccgg

SEQ ID NO: 58 - DNA, 20 bp, artificial
  synthetic oligonucleotide primer
tttaccctgctgagctctcc

SEQ ID NO: 59 - DNA, 20 bp, artificial
  synthetic oligonucleotide primer
gtggaagctgcaaggtcttc

SEQ ID NO: 60 - DNA, 20 bp, artificial
  synthetic oligonucleotide primer
tatttgatagggccttgcca

SEQ ID NO: 61 - DNA, 20 bp, artificial
  synthetic oligonucleotide primer
cctcctctgttccaaacagg

SEQ ID NO: 62 - DNA, 20 bp, artificial
  synthetic oligonucleotide primer
ttctgtcagtcccattggtg

SEQ ID NO: 63 - DNA, 20 bp, artificial
  synthetic oligonucleotide primer
tgtccatcttgtcgtcttcg

SEQ ID NO: 64 - DNA, 20 bp, artificial
  synthetic oligonucleotide primer
tgtgtctagagcatggctgg

SEQ ID NO: 65 - DNA, 20 bp, artificial
  synthetic oligonucleotide primer
gcaattgcaagcatctcaaa

SEQ ID NO: 66 - DNA, 20 bp, artificial
  synthetic oligonucleotide primer
caaccccaacgtcaaagtct

SEQ ID NO: 67 - DNA, 20 bp, artificial
  synthetic oligonucleotide primer
cacctccttgtcaaccctgt

SEQ ID NO: 68 - DNA, 20 bp, artificial
  synthetic oligonucleotide primer
gaaaggatcttgggcacttg

SEQ ID NO: 69 - DNA, 20 bp, artificial
  synthetic oligonucleotide primer
cggcagatacaatgtgatgg

SEQ ID NO: 70 - DNA, 20 bp, artificial
  synthetic oligonucleotide primer
ttccaaacctgctgataggg

SEQ ID NO: 71 - DNA, 20 bp, artificial
  synthetic oligonucleotide primer
ctacccagatcttttgccca

SEQ ID NO: 72 - DNA, 20 bp, artificial
  synthetic oligonucleotide primer
caacccacagaatgagagca

SEQ ID NO: 73 - DNA, 21 bp, artificial
  synthetic oligonucleotide primer
ggccttgtagaatgagtttgg

SEQ ID NO: 74 - DNA, 20 bp, artificial
  synthetic oligonucleotide primer
tgaaaagtctccccagcagt

SEQ ID NO: 75 - DNA, 20 bp, artificial
  synthetic oligonucleotide primer
ttgcttggagagtttcgtcc

SEQ ID NO: 76 - DNA, 20 bp, artificial
  synthetic oligonucleotide primer
gggagacaggaaagaaggaa

SEQ ID NO: 77 - DNA, 20 bp, artificial
  synthetic oligonucleotide primer
ccattgggactgtgctaggt

SEQ ID NO: 78 - DNA, 20 bp, artificial
  synthetic oligonucleotide primer
ctggcttcaccactgactga

SEQ ID NO: 79 - DNA, 20 bp, artificial
  synthetic oligonucleotide primer
tctcccctctcctgtcttca

SEQ ID NO: 80 - DNA, 20 bp, artificial
  synthetic oligonucleotide primer
ggggaacatgcgacctagta

SEQ ID NO: 81 - DNA, 20 bp, artificial
  synthetic oligonucleotide primer
aggggttcatgtgcatgttt

SEQ ID NO: 82 - DNA, 20 bp, artificial
  synthetic oligonucleotide primer
ttgaggttgtagggtgggag

SEQ ID NO: 83 - DNA, 20 bp, artificial
  synthetic oligonucleotide primer
cagtgagcccctttcttctg

SEQ ID NO: 84 - DNA, 20 bp, artificial
  synthetic oligonucleotide primer
ggccaggcaaaagaatatca

SEQ ID NO: 85 - DNA, 20 bp, artificial
  synthetic oligonucleotide primer
ggtttggaggaggaaggaag

SEQ ID NO: 86 - DNA, 20 bp, artificial
  synthetic oligonucleotide primer
gcagatccccaacagtccta

SEQ ID NO: 87 - DNA, 20 bp, artificial
  synthetic oligonucleotide primer
tcctgctttctgccattctt

SEQ ID NO: 88 - DNA, 20 bp, artificial
  synthetic oligonucleotide primer
cttgggacacagtgaaccct

SEQ ID NO: 89 - DNA, 20 bp, artificial
  synthetic oligonucleotide primer
taaatggtctggcaactccc

SEQ ID NO: 90 - DNA, 20 bp, artificial
  synthetic oligonucleotide primer
cttggacacagctccacaga

SEQ ID NO: 91 - DNA, 20 bp, artificial
  synthetic oligonucleotide primer
tgggtgacagaggaagatcc

SEQ ID NO: 92 - DNA, 20 bp, artificial
  synthetic oligonucleotide primer
tgctctcatctgtgtctggc

SEQ ID NO: 93 - DNA, 20 bp, artificial
  synthetic oligonucleotide primer
ctgtgaccagggagaatggt

SEQ ID NO: 94 - DNA, 24 bp, artificial
  synthetic oligonucleotide primer
ttcagattaccaagtttcttcagc

SEQ ID NO: 95 - DNA, 24 bp, artificial
  synthetic oligonucleotide primer
ttgaggacttcagcctttcaccgc

SEQ ID NO: 96 - DNA, 20 bp, artificial
  synthetic oligonucleotide primer
gtgcagcagtcacctgagaa

SEQ ID NO: 97 - DNA, 20 bp, artificial
  synthetic oligonucleotide primer
gggtacgtgtgcacaacttg

SEQ ID NO: 98 - DNA, 20 bp, artificial
  synthetic oligonucleotide primer
agctgcaggtctgttggagt

SEQ ID NO: 99 - DNA, 20 bp, artificial
  synthetic oligonucleotide primer
gccttgaaggtcctttccat

SEQ ID NO: 100 - DNA, 1433 bp, homo sapiens
ggactgaagtttgatatgatgttccgatgtcattggttttgtcgtactatgaccatggttttgtctctatatctagttaccttgtcttgtctcgggagtctttattacgacgcatagatgttggtagactagaaactgtttggactgtttttgttctttaacccttttctaagggataaattgtttaccacgacacttttgaccaatcggtatacatctttcgactttgacctagggaaggaatgtggaatatctttttaattaagttctacctaagttctgaatttacaatctggattttggtattttgggatcttcttttggatccgttatggtaagtactgtatccgtggccgttcctgaagtacagattttgtggttttcgttaccgttgttttcggttttaactgtttaccctagattaatttgatttctcgaagatgtgtcgttttctttgatggtagtctcacttgtccgttggatgtcttaccctcttttaaaaaaggtagatgagtagactgtttcccgtttatgggtcttagatgtttcttgagtttgtttaaatgttctttttttgtttgttggggtagtttttcacccgtctcctataattgtctatgaagagtttttttctgtaaatacgtcggttgtctgtgtacttttttacgagtagtagtgaccggtagtctctttacgtttagttttggtgttactctatggtagagtgtggtcaatcttaccgctagtaatttttcagtcctttgttgtccacgacctttcctacacctctttatccttgtgaaaatgtgacaaccaccctgacatttgatcaagttggtaacaccttcagtcacaccactaaggagtccctagatcttgatctttatggtaacgtgggtcggtagggtaataacccatatatgggtttcctaatatacagtacgacgagatttccatgtacgtgtgtatacaaataacaccgtgataagtgttatcgtttcggaaccttggttgggtttacaggtagttactatctgacctaattcttttacaccgtatatatgtggtaccttatgatacgtcggtattttttactactcaagtacaggaaacatccctgtacctacttcgacctttggtagtaagagtcgtttgatagagttcctgtttttcggtttgtgacgtacaagagtgagtgtccacccttaacttgttactcttgtggacctgtgtccttccccttgtagtgtgtgaccccggacaacaccccaaccccttcccacctccctatcgtaatcctctatatgaattacatttattcctcaattacccacgtcgtgtggttgaaccgtgtacatatgtatacattgtttggacgttcaacacgtgtgcatgggatcttgaatttcatattattttttataaattttttt

SEQ ID NO: 101 - DNA, 1394 bp, homo sapiens
tatactttaagttctgggatacatgtgcagaacgtgcaggtttcttacataggtatactcgtgccatggtggtttgctgcacccatcaacctgccatatacattaagtatttctcctaatgctatctttcccctagccctaccccactccctgacaggccctggtctatgatgttcccctccctgtgtccatgtgttctcattgttcaactgccacttatgagtgagaacatgtggtgtttggttttctgttcttgtgttttagtttgctgaggatgatggtttccagcttcatccatgtccctgcaaaggacatgaactcatcctttttgatggctgcatagtattccatggtgtatatgtgccacgttttctttatccagtatatcattgatgggcattttggttggttccaagtctttgctattgtgaatagtgctgcaataaacatacgtgtgcatttgtctttatagaagaatgatttataatcttttgggtatatacccagtaatgggattgctgagtcaaatgatatttctggttctagatccttaatgaattgccacactgtcttccacaatggttgaactaatttatgctcccaccaacagtgtaaaagcgttcctatttcttcaaatcctcaccagcatctgttgtttcctgactttttaatcgccattctaactggcatgagatggtatctcattgtggttttgatttgcatttctctaatgaccagtgatgatgagctttttttcatgtttgttggcagcataaatgtcttcttttgagaagtgtctgttcatattcttcacccactttttgatggagttatttgttttcttcttgtaaatttgtttaagttccttgtcgattctggatattagctctttgtcagatgaatagattgcaaaaattttctcccattctgtaagttgcctgttccctctgctgatagtttcttctgctgtgcagaagctctttagtttaattagatcccatttgtcaattttggcttttgttgccattgcttctggtgttttagtcatgaagtctctacccatgcctatgtcctggatggtattgccttggttttcttctacagtttttatggttttaggtcttgcatttaagtctttaatccatcttgagttaattttgtataacgtgtaaggaagaggtccactttcagttttctgcatgaggctaacgagttttcccaacaccatttattaaatagggaatcctttccccattgtttgtttttgtcaagtttgtcaaagatcaggtggttgtagatgtgtggtgttatttctgaggcctctgctctgttccacgtgtctatatctctgttttggtaccagtaccatgctgttttgggtactgtaccacttg

SEQ ID NO: 102 - protein, 16 aa, homo sapiens
RKCYEAMTLGARKLKK

SEQ ID NO: 103 - protein, 8 aa, artificial
  synthetic stop codon generating a 1/2/3 AR mutant
CYEAMTLG

SEQ ID NO: 104 - protein, 30 aa, homo sapiens
RKCYEAMTLGAAVVVSERILRVFGVSEWLP

SEQ ID NO: 105 - protein, 16 aa, homo sapiens
RKCYEAMTLGAGSRVS

SEQ ID NO: 106 - protein, 26 aa, homo sapiens
RKCYEAMTLGEKFRVGNCKHLKMTRP

SEQ ID NO: 107 - protein, 21 aa, homo sapiens
RKCYEAMTLGGFFRMNKLKES

SEQ ID NO: 108 - protein, 34 aa, homo sapiens
RKCYEAMTLGARKLKKLGNLKLQEEGEASSTTSP

(19) United States
(12) Patent Application Publication
(10) Pub. No.: US 2013/0130241 A1
(43) Pub. Date: May 23, 2013

(54) ANDROGEN RECEPTOR ISOFORMS AND METHODS

(71) Applicant: Scott M. Dehm, Plymouth, MN (US)

(72) Inventor: Scott M. Dehm, Plymouth, MN (US)

(73) Assignee: Regents of the University of Minnesota, St. Paul, MN (US)

(21) Appl. No.: 13/582,600

(22) PCT Filed: Mar. 8, 2011
(86) PCT No.: PCT/US2011/027545

§ 371 Date: Dec. 10, 2012

Related U.S. Application Data
(60) Provisional application No. 61/311,603, filed on Mar. 8, 2010.

(51) Int. Cl. C12Q 1/68

(52) U.S. Cl. CPC C12Q 1/6876
USPC 435/6.11

(57) ABSTRACT

The invention relates to exploiting differences in androgen receptor gene amplification and expression of androgen receptor isoforms in various cell types such as, for example, prostate tumor cells. In one aspect, the invention provides a method for detecting unbalanced amplification of androgen receptor isoforms. Generally, the method includes receiving a biological sample obtained from a subject, the biological sample comprising cells expressing a plurality of non-wild-type androgen receptors, measuring the copy number of at least a polynucleotide encoding a first non-wild-type androgen receptor and a polynucleotide encoding a second non-wild-type androgen receptor, thereby producing an expression ratio, and identifying the sample as exhibiting unbalanced amplification of androgen receptor if the expression ratio is no less than a predetermined expression ratio. In another aspect, the invention provides a method of analyzing a biological sample from a subject. Generally, the method includes receiving the biological sample, the biological sample comprising cells expressing a plurality of androgen receptor isoforms, measuring expression of at least one androgen receptor isoform, and identifying the sample as exhibiting a predetermined pattern of androgen receptor isoform expression if at least one predetermined pattern of androgen receptor expression is detected.

### PRIORITY INFORMATION

This application claims the benefit of U.S. Provisional Application Ser. No. 61/311,603, filed Mar. 8, 2010, which is incorporated by reference herein.

### GOVERNMENT FUNDING

The present invention was made with government support under Grant No. PC094384, awarded by the Department of Defense Prostate Cancer Research Program. The Government has certain rights in this invention.

### BACKGROUND

The androgen receptor (AR) is a steroid hormone receptor transcription factor that mediates target gene activation in response to the androgens testosterone and dihydrotestosterone (DHT). The androgen receptor has an overall modular organization similar to other steroid receptors (FIG. 1), and is composed of an N-terminal domain (NTD) harboring androgen receptor transcriptional activation function (AF)-1, a central 2-zinc finger DNA binding domain (DBD), a short hinge region, and a COOH-terminal domain (CTD), which contains both the androgen receptor ligand-binding domain (LBD) and AF-2 co-activator binding surface (Bain et al., 2006 Annu Rev Physiol 69). The large and unordered androgen receptor NTD has been recalcitrant to structural determination. Conversely, the three-dimensional structures of the androgen receptor DBD and CTD modules have been solved, and are highly similar to the DBD and CTD modules of other steroid receptors (He et al., 2004 Mol Cell 16:425-38; Matias et al., 2000 J Biol Chem 275:26164-71). Mechanistically, transcriptional activation by the androgen receptor relies on engagement of co-regulator proteins with functional domains in both the androgen receptor NTD and CTD. To date, over 150 discrete androgen receptor-associated co-regulatory proteins have been described (Heemers and Tindall, 2007 Endocr Rev 28:778-808). Some of these co-regulators, such as SRC-1, SRC-2, and SRC-3, have been shown to engage with androgen receptor AF-2, but can also bind in an alternative fashion to the androgen receptor NTD (Bevan et al., 1999 Mol Cell Biol 19:8383-92). Such findings have stirred debate as to whether the androgen receptor AF-2 domain is the primary mediator of transcriptional activation, or whether androgen receptor AF-1 plays a dominant role. Regardless of these mechanistic considerations, the large, multi-protein complexes nucleated by the androgen receptor serve to recruit and activate the basal transcriptional apparatus, which results in a finely-tuned level of target gene transcription (Naar et al., 2001 Annu Rev Biochem 70:475-501).

Prostate cancer (PCa) is an androgen and androgen receptor-dependent disease. Medical/chemical castration and androgen receptor antagonism with anti-androgens such as bicalutamide are treatment modalities for recurrent or metastatic prostate cancer. The length of time the disease is controlled by these so-called androgen depletion therapies varies, with median ranges from 18-33 months (Scherr et al., 2003 Urology 61:14-24). Prostate cancer that has relapsed post-androgen depletion is referred to as androgen-refractory, androgen-independent, or more appropriately androgen depletion-independent (ADI) by virtue of a acquired ability to grow despite a castrate level of androgens (Roy-Burman et al., 2005 Cancer Biol Ther 4:4-5). It is now apparent that adaptive mechanisms employed by the androgen receptor may be involved in this disease progression (Grossmann et al., 2001 J Nat Cancer Inst 93:1687-97; Litvinov et al., 2003 J Clin Endocrinol Metab 88:2972-82). Indeed, most ADI tumors retain androgen receptor expression and also express and secrete prostate specific antigen (PSA), a well-characterized androgen receptor-regulated gene (van der Kwast et al., 1991 Int J Cancer 48:189-93; van der Kwast and Tetu, 1996 Eur Urol 30:265-8; Hobisch et al., 1995 Cancer Res 55:3068-72; Hobisch et al., 1996 Prostate 28:129-35; Sadi et al., 1991 Cancer 67:3057-64; Tilley et al., 1994 Cancer Res 54:4096-102). In a model system, androgen-dependent prostate cancer xenografts exhibit loss of androgen receptor nuclear staining and function upon castration of male hosts, but soon progress to an ADI stage through aberrant re-acquisition of nuclear androgen receptor expression and activity (Zhang et al. 2003 Cancer Res 2003; 63:4552-60). In addition, disruption of androgen receptor function inhibits proliferation and PSA expression in both androgen-dependent and ADI prostate cancer cells (Zegarra-Moro et al., 2002 Cancer Research 62:1008-13; Liao et al., 2005 Mol Cancer Ther 4:505-15; Agoulnik et al., 2005 Cancer Res 65:7959-67; Haag et al., 2005 J Steroid Biochem Mol Biol 24:24; Wu et al., 1994 Int J Cancer 57:406-12).

### SUMMARY OF THE INVENTION

In one aspect, the invention provides a method for detecting unbalanced amplification of a polynucleotide sequence that encodes an androgen receptor. Generally, the method includes receiving a biological sample obtained from a subject, the biological sample comprising cells expressing a plurality of non-wild-type androgen receptors, measuring the copy number of at least a polynucleotide encoding a first non-wild-type androgen receptor and a polynucleotide encoding a second non-wild-type, androgen receptor, thereby producing an expression ratio, and identifying the sample as exhibiting unbalanced amplification of androgen receptor if the expression ratio is no less than a predetermined expression ratio.

In some embodiments, the first non-wild-type androgen receptor can include Exon 3. In some embodiments, the second non-wild-type androgen receptor can include Exon 8.

In some embodiments, the expression ratio is no less than 1.5:1. In some embodiments, the method further includes identifying the subject as at risk for androgen depletion-independent prostate cancer. In some embodiments, the method can include either initiating or modifying treatment of the subject based on detecting unbalanced amplification of a polynucleotide that encodes an androgen receptor. In some of these embodiments, initiating or modifying treatment can include administering to the subject at least one pharmaceutical composition effective for treating androgen depletion-independent prostate cancer.

In another aspect, the invention provides a method of analyzing a biological sample from a subject. Generally, the method includes receiving the biological sample, the biological sample comprising cells expressing a plurality of androgen receptor isoforms, measuring expression of at least one androgen receptor isoform, and identifying the sample if the measured androgen receptor isoform expression comprises at least one of the following: wild-type isoform is no more than a predetermined percentage of total androgen receptor isoform expression, isoform 1/2/3/2b is expressed as a greater percentage of total androgen receptor isoform expression than is observed in a normal control, or isoform 1/2/3/CE3 is expressed as a greater percentage of total androgen receptor isoform expression than is observed in a normal control.

In some embodiments, the method further includes identifying the subject as at risk for androgen depletion-independent prostate cancer. In some embodiments, the method can include either initiating or modifying treatment of the subject based on detecting unbalanced amplification of a polynucleotide that encodes an androgen receptor. In some of these embodiments, initiating or modifying treatment can include administering to the subject at least one pharmaceutical composition effective for treating androgen depletion-independent prostate cancer.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a schematic of an androgen receptor (AR) dimer bound to an androgen response element (ARE), SEQ ID NOs: 36 and 37. The modular domain organization of the androgen receptor is indicated.

FIG. 2 is a scale diagram of the ˜180 kb AR locus. Exon 1 encodes the androgen receptor NTD/AF-1 domain, Exons 2 and 3 each encode one zinc finger of the two zinc finger androgen receptor DBD, and Exons 4-8 encode the androgen receptor LBD/AF-2 module. The locations of novel exons in the androgen receptor locus, termed Exon 2b, CE1, CE2, and CE3 are shown. Splicing of these exons after AR Exon 3 gives rise to androgen receptor isoforms containing the NTD/AF-1 and DBD modules, but lacking the LBD/AF-2 module.

FIG. 3. A) Schematic of androgen receptor mRNA isoforms expressed in 22Rv1 cells. DBD*, Exon 3-duplicated AR. B) 22Rv1 cells were electroporated with siRNAs targeted to Exons 1, 2, or 4. androgen receptor and ERK-2 levels were determined. C) Rabbit antisera were raised against an Exon 2b-derived peptide. Equal amounts of 22Rv1 lysates were immunoprecipitated with crude antisera, or antibody purified on an immobilized Exon2b-peptide column, non-specific rabbit IgG, or a rabbit polyclonal antibody specific for the androgen receptor NTD. Western blots were performed with a mouse monoclonal antibody specific for the androgen receptor NTD.

FIG. 4. Expression constructs encoding androgen receptor isoforms (1/2/312b, SEQ ID NO: 38; 1/2/3/CE1, SEQ ID NO: 39; 1/2/3/CE2, SEQ ID NO: 40; 1/2/3/CE3, SEQ ID NO: 41, and wild type AR, SEQ ID NO: 42) shown in (A) were tested in transient transfection experiments in AR-null DU145 cells (B). Data represent the mean+/−S.E.M. from 3 independent experiments, each performed in duplicate.

FIG. 5. (A) Bidirectional lentivirus expression system. 22Rv1 cells were transduced with the indicated lentiviruses. (B) ADI growth was determined by crystal violet staining and (C) androgen receptor expression was assessed by Western blot.

FIG. 6. (A) Locations of PCR amplicons along the AR locus. (B, D) Quantitative genomic PCR was used to determine copy number for amplicons depicted (A). For LuCaP 35 xenografts, 5q22.2 and 21q21.3 amplicons were also quantified to verify an AR-specific increase in copy number. (C,E) Expression of androgen receptor isoforms was assessed by Western blot. (F) Expression of 1/2/3/2b and full-length androgen receptor mRNAs were determined by RT-PCR using primer sets indicated.

FIG. 7. Specificity of androgen receptor isoform-targeted PCR primers. Standard templates representing individual androgen receptor isoforms were amplified with the indicated PCR primer sets.

FIG. 8. Efficient and stable synthesis of alternatively spliced AR mRNA isoforms in CRPCa cells. A, Growth of CWR22Pc and 22Rv1 cells in the presence or absence of androgens. B, Plasmid templates harboring depicted cDNAs were subjected to PCR with indicated primer pairs. Right panels, mRNA from CWR22Pc and 22Rv1 cells was subjected to quantitative RT-PCR using indicated primer sets. Ct values obtained from qRT-PCR reactions were converted to copy number by plotting sample Ct values on Ct vs. copy number standard curves constructed from concurrent qPCR analysis of serial dilutions of plasmid templates. Data represent Mean+/−Standard Error from two independent experiments, each performed in triplicate (n=6). C, AR Western blots of CWR22Pc and 22Rv1 cells following 3 day treatment with 1 nM DHT. ERK-2, loading control. D, AR Western blot of 22Rv1 cells following 10 day culture in steroid depleted medium containing 1 nM DHT or vehicle control. ERK-2, loading control.

FIG. 9. Alternatively spliced AR exons are contained on a rearranged genomic segment in 22Rv1 cells. A, Schematic of the ˜180 kb AR locus at Xp11-12. PCR amplicons used for copy number determination are labeled A-F. B, Genomic DNA from CWR22Pc and 22Rv1 cells was subjected to quantitative PCR using amplicon primer pairs indicated in A. Ct values were converted to copy number by plotting sample Ct values on Ct vs. copy number standard curves constructed from serial dilutions of BPH-1 genomic DNA. Data represent Mean+/−Standard Error from two independent experiments, each performed in triplicate (n=6).

FIG. 10. AR intragenic rearrangements in CRPCa detected by Affymetrix Genome Wide SNP 6.0 Array analysis of metastatic tissues. Top, Exon organization of the AR locus on Xq11-12 and chromosome position (human genome build 19, hg19) is indicated at the top of each panel. All panels shown are individual tissue samples from CRPCa metastases. Blue dots represent probe-level copy number, horizontal red lines represent mean segment copy number, horizontal green dashed lines represent standard deviation, and dashed vertical lines represent segment boundaries defined by the segmentation algorithm. Black horizontal lines with downward-facing arrowheads denote a region of focal copy number alteration similar to 22Rv1 cells.

FIG. 11. Fine mapping of AR intragenic rearrangement segment boundaries in 22Rv1 cells. A, Schematic of the AR locus at Xq11-12. PCR amplicons used for copy number determination are labeled B, C, E, F, and G-S. B, Genomic DNA from 22Rv1 cells was subjected to quantitative PCR using amplicon primer pairs indicated in A. Ct values were converted to copy number by plotting sample Ct values on Ct vs. copy number standard curves constructed from serial dilutions of BPH-1 genomic DNA. C, Schematic of repetitive element organization at the 5′ and 3′ boundaries of the 22Rv1 duplicated AR segment in the reference human genome. Elements were defined by RepeatMasker 3.0 (Tarailo-Graovac and Chen, 2009 Curr Protoc Bioinformatics Chapter 4: Unit 4 10). Black arrows indicate the directional orientation of L1 elements. L1 elements are named based on their evolutionary origin and sequence divergence, with relative ages (oldest to youngest) L1M1>L1MA3>L1PB2>L1PREC2>L1PA7>L1PA5 (Tarailo-Graovac and Chen, 2009 Curr Protoc Bioinformatics Chapter 4: Unit 4 10; Khan et al., 2006 Genome Res 16:78-87).

FIG. 12. Outward-facing PCR to isolate the 22Rv1 AR tandem duplication. A, Schematic of the AR locus at Xq11-12 with locations of primers used for outward-facing long-range PCR. B, Schematic of the AR locus in 22Rv1 cells as revealed by sequencing of cloned long-range PCR products. C, Electropherogram sequence of the AR break fusion junction in 22Rv1 cells, including a novel 27 bp insert (SEQ ID NO: 43). D, Sequence alignments the 3′ breakpoint (SEQ ID NO: 44), the 22Rv1 break fusion junction (SEQ ID NO: 45), and the 5′ breakpoint (SEQ ID NO: 46). Sequence contained in the break fusion junction is shaded in gray. Regions of microhomology are boxed.

FIG. 13. Concurrent emergence of AR intragenic rearrangement, androgen-independent growth, and high-level truncated AR isoform expression during CWR22Pc castration. A, Schematic of the 22Rv1 AR locus and locations of primers used for nested PCR. B, Conventional PCR was performed using Tfwd/Trev primers and 40 ng of input DNA from the indicated cell lines. An aliquot of this reaction was used in a second nested PCR reaction using Ufwd/Trev primers. C, AR Western blot of CWR22Pc castration time-course. CWR22Pc cells were cultured in androgen-depleted medium for the indicated time-points. ERK-2, loading control. D, Nested PCR of CWR22Pc castration time-course. Reactions were performed exactly as described in B.

FIG. 14. AR protein expression patterns in CWR22Pc and 22Rv1 cells. Lysates from CWR22Pc and 22Rv1 cells were analyzed by Western blot analysis using antibodies specific for the AR NTD and AR 1/2/3/CE3 (also known as ARV-7). ERK2, loading control.

FIG. 15. Relative expression changes in detectable AR mRNA isoforms by RT-PCR following treatment of 22Rv1 cells with 1 nM DHT for 0, 24, or 72 h. Values are shown relative to GAPDH and were calculated using the 2^{-ΔΔCt} method. GAPDH threshold cycle of amplification (Ct) values were not altered by androgen treatment. Data represent Mean+/−Standard Error from two independent experiments, each performed in triplicate (n=6).

FIG. 16. Affymetrix Genome Wide SNP 6.0 Array analysis of 58 metastatic tissues from 14 rapid autopsy subjects. A-N, All panels in A are individual metastatic CRPCa tissue samples from Subject 3, panels in B are from Subject 12, panels in C are from Subject 16, panels in D are from Subject 17, panels in E are from Subject 19, panels in F are from Subject 21, panels in G are from Subject 22, panels in H are from Subject 24, panels in I are from Subject 28, panels in J are from Subject 30, panels in K are from Subject 31, panels in L are from Subject 32, panels in M are from Subject 33, and panels in N are from Subject 34. Anatomic sites of metastases for all 58 coded tumor samples are detailed in (Liu et al., 2009 Nat Med 15:559-65) and the GEO website (accession GSE14996). Dots represent probe-level copy number, horizontal solid lines represent mean segment copy number, horizontal dashed lines represent standard deviation, and dashed vertical lines represent segment boundaries defined by the segmentation algorithm.

FIG. 17. Increased Exon 3 segment content vs. Exon 4 segment content in a subset of CRPCa metastases. Affymetrix SNP6.0 array copy number data from datasets GSE14996 (Liu et al., 2009 Nat Med 15:559-65) and GSE18333 (Mao et al., 2010 Cancer Res 70:5207-12) was segmented, and the mean copy number of the segment harboring Exon 3 was divided by the mean copy number of the segment harboring Exon 4. Grey bars denote metastases with at least a 20% increase in Exon 3 vs. Exon 4 segment content. Grey numbers denote patients with at least one tumor displaying a 20% increase in Exon 3 vs. Exon 4 segment content. Fisher's exact test determined that patients with CRPCa are more likely to display increased Exon 3 vs. Exon 4 segment content than patients with primary PCa (5/14 vs. 0/44, P=0.000437).

FIG. 18. Affymetrix Genome Wide SNP 6.0 Array analysis of normal tissue DNA from 6 rapid autopsy subjects. Blue dots represent probe-level copy number, horizontal red lines represent mean segment copy number, horizontal green dashed lines represent standard deviation, and dashed vertical lines represent segment boundaries defined by the segmentation algorithm.

FIG. 19. Confirmation of AR intragenic copy number alterations in a CRPCa rapid autopsy subject by real-time genomic PCR. A, Schematic of the AR locus at Xq11-12. PCR amplicons used for copy number determination are labeled B-E and S. B, Genomic DNA from separate metastases was subjected to quantitative PCR using amplicon primer pairs indicated in A. Ct values were converted to copy number by plotting sample Ct values on Ct vs. copy number standard curves constructed from serial dilutions of BPH-1 genomic DNA. Data represent Mean+/−Standard Error from two independent experiments, each performed in triplicate (n=6). *P<0.05; **P<0.01. Although these data indicate copy number of 5 for amplicon D, and copy number of 4 for amplicons E and S in the temporal subdural metastasis, student's t-tests for differences between D and E or D and S did not reach statistical significance. C, Corresponding plots derived from Affymetrix SNP6.0 analysis. Dots represent probe-level copy number, horizontal solid lines represent mean segment copy number, horizontal dashed lines represent standard deviation, and dashed vertical lines represent segment boundaries defined by the segmentation algorithm. The segmentation algorithm only detected 1 of 2 breakpoints encompassing AR Exons 2b-3 in a pulmonary hilar lymph node metastasis.

FIG. 20. ClustalW pairwise sequence alignment of conserved LINE-1 elements at the 5′ and 3′ AR break junctions. The L1PA7 LINE-1 element (SEQ ID NO: 101) located in the LINE-1 cluster at the 5′ AR break junction and the L1PA5 LINE-1 element (SEQ ID NO: 100) at the 3′ AR break junction were aligned using the ClustalW alignment program of the MacVector software package. The positions of LINE-1 elements were defined by RepeatMasker 3.0 (Tarailo-Graovac and Chen, 2009 Curr Protoc Bioinformatics Chapter 4: Unit 4 10). Black arrows indicate the directional orientation of L1 elements. L1 elements are named based on their evolutionary origin and divergence in sequence from a transposition-competent L1 element, with relative ages (oldest to youngest) L1PA7>L1PA5 (Tarailo-Graovac and Chen, 2009 Curr Protoc Bioinformatics Chapter 4: Unit 4 10; Khan et al., 2006 Genome Res 16:78-87).

FIG. 21. A, Schematic of the 22Rv1 AR locus and locations of primers used for long-range PCR. B, Long-range PCR was performed using Qfwd/Krev and Qfwd/Jrev primers and 200 ng of input DNA from the indicated cell lines. C, Conventional PCR was performed using Tfwd/Trev and Ufwd/Trev primers and 40 ng of input DNA from the indicated cell lines. D, 22Rv1 nested PCR was performed on a 1 μL aliquot from an initial Tfwd/Trev PCR reaction using Ufwd/Trev primers as indicated. Nested PCR displayed detection sensitivity as low as 5 pg DNA per reaction, which is approximately equal to the mass of a single diploid cell genome.

FIG. 22 shows the relationship between coding exons and AR protein domains.

FIG. 23. Organization of the AR locus and relationship between exons and truncated AR isoforms. Alternative splicing via cryptic exon (grey) inclusion or exon skipping gives rise to truncated AR isoforms that lack the AR LBD and contain short unique CTD sequences.

FIG. 24. High-level synthesis of alternatively spliced, truncated AR isoforms in CRPCa 22Rv1 cells. (A) Growth of CWR22Pc and 22Rv1 cells in the presence or absence of androgens. (B) AR isoform-specific primer sets were used in qRT-PCR reactions to derive Ct values, which were converted to copy number by plotting on Ct vs. copy number standard curves constructed with plasmid template standards.

FIG. 25. Intragenic AR tandem duplication in 22Rv1 cells. (A) AR copy number in CWR22Pc and 22Rv1 cells was determined via qPCR. (B) Schematic of AR gene structure in 22Rv1 cells based on high-resolution copy number analysis and break fusion junction cloning. hg19 numbering is used.

FIG. 26. AR gene structure in representative metastases from 4 rapid autopsy subjects. Affymetrix SNP6.0 probe-level copy number data (dots) was used as input in a segmentation algorithm (solid lines, mean segment copy number, dashed lines, standard deviation). Focal copy number increase for AR Exon 3 is indicated by arrowheads.

FIG. 27. Multiplex Ligation-Dependent Probe Assay (MLPA) for high-throughput AR copy number determination in clinical PCa specimens. (A) AR locus schematic with MLPA probe locations. Most loci are interrogated with independent A and B probe sets. (B) Example electropherogram of resolved MLPA amplification products. (C) Electropherogram peak areas are normalized to derive copy number. X-chromosome control probes are used to verify locus-specific copy number changes.

FIG. 28. Absolute quantification RT-PCR with RNA from FFPE tissue.

FIG. 29. a custom-designed Agilent SureSelect AR bait library of ˜1500×120 bp RNA oligonucleotides for AR sequence capture is visualized using the UCSC Genome Browser based on RefSeq, UniProt, GenBank, CCDS, and Comparative Genomics. Three gaps in the library arising due to extended repeats defined by RepeatMasker are shown.

FIG. 30. A model of interactions in AR signaling.

FIG. 31. Localization and activity of AR isoforms. (A) Sequences of unique COOH-terminal extensions 1/2/3/CE1 (SEQ ID NO: 104), 1/2/3/CE2 (SEQ ID NO: 105), 1/2/3/CE3 (SEQ ID NO: 106), 1/2/3/2b (SEQ ID NO: 107), and wild type AR (SEQ ID NO: 108). (B) Fractionation of nuclear (N) and cytoplasmic (C) compartments in electroporated LNCaP cells. (C) Activity of a PSA-LUC reporter construct following AR knock-down and reexpression of siRNA-resistant AR isoforms (denoted AR(sr)) in LNCaP cells.

FIG. 32. (A) Bidirectional lentivirus vector schematic. Expression of shRNA and shRNA-resistant genes are controlled by the histone H1 and EF-1α promoters, respectively. Refer to FIG. 22 for expected isoform targeting by shRNAs. LNCaP and 22Rv1 cells were infected with lentiviral vectors and subjected to (B) Western blot or (C) light and fluorescence microscopy 10 days post-infection.

FIG. 33. CWR22Pc model of PCa progression. (A) Schematic of outward facing PCR assay for detecting the 22Rv1 break fusion junction (B) CWR22Pc cells were cultured for 32 days under castrate conditions. (C) Model of CWR22Pc progression to a CRPCa phenotype during long-term castration.

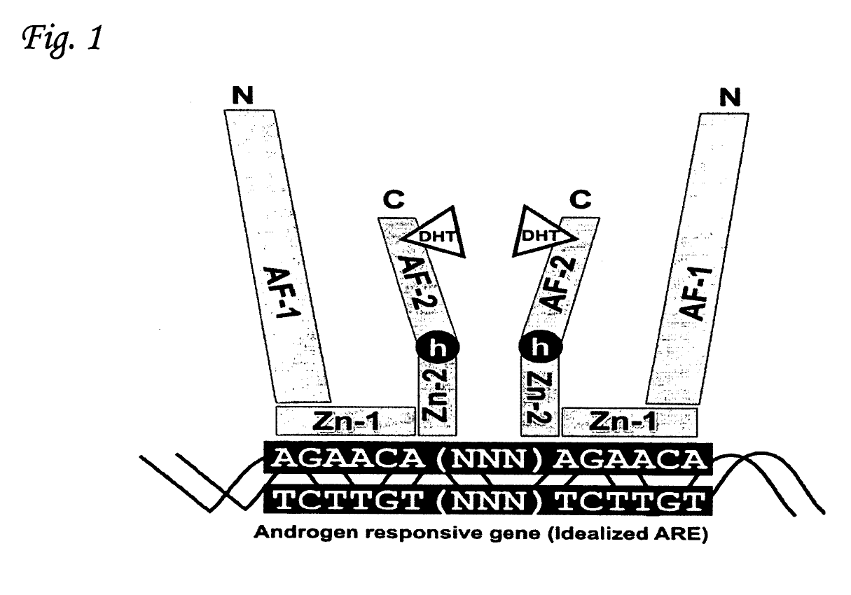

### DETAILED DESCRIPTION OF ILLUSTRATIVE EMBODIMENTS

In various aspects, the present invention provides PCR primers, template standards, and methods for measuring and calculating the copy number of alternatively spliced androgen receptor isoforms. Thus, the invention can permit one to measure and quantify the amounts of wild-type and alternatively spliced androgen receptor isoforms in prostate cancer cell lines and tissues. These measurements can help guide treatment decisions by predicting the likelihood that a particular subject may respond or be resistant to prostate cancer therapies targeted to the androgen receptor. Thus, in some cases, methods described herein may be used to identify subjects under treatment for prostate cancer as at risk for developing androgen depletion-independent prostate cancer. Such an evaluation may indicate that a change in prescribed therapy is appropriate. In some of these instances, the change may involve modifying the subject's treatment regimen to include administration of a pharmaceutical composition effective for treating androgen depletion-independent prostate cancer.

The methods described herein can provide an absolute quantitation strategy to permit copy number calculations for alternatively spliced androgen receptor isoforms. Previous studies by other groups have employed qRT-PCR to derive Ct (threshold cycle of amplification) values for these androgen receptor isoforms, and then used the 2^{-ΔΔCt} method to calculate relative abundance. This approach is incorrect and an inappropriate application of the 2^{-ΔΔCt} formula. In contrast, methods described herein involve using PCR template standards to derive Ct vs. copy number standard curves. We then derive Ct values from an experimental source such as, for example, a cell line, a tissue sample, etc., and extrapolate against this curve to generate copy number calculations. The methods described herein are the only methods that allow one to compare both the absolute and relative levels of alternatively spliced androgen receptor isoforms in prostate cancer.

In the description that follows, the term “and/or” means one or all of the listed elements or a combination of any two or more of the listed elements; the terms “comprises” and variations thereof do not have a limiting meaning where these terms appear in the description and claims; and unless otherwise specified, “a,” “an,” “the,” and “at least one” are used interchangeably and mean one or more than one. Also herein, the recitations of numerical ranges by endpoints include all numbers subsumed within that range (e.g., 1 to 5 includes 1, 1.5, 2, 2.75, 3, 3.80, 4, 5, etc.).

Systemic therapy for prostate cancer involves inhibiting the activity of the androgen receptor. LHRH analogs (e.g., LUPRON, Abbott Laboratories, Abbott Park, Ill.) and anti-androgens (e.g., bicalutamide) are used to inhibit androgen receptor activity, and result in clinical response. A limitation of this so-called androgen depletion therapy is that prostate cancer can eventually recur in a lethal form referred to as castration-resistant prostate cancer. Clinical and experimental research have demonstrated that castration-resistant prostate cancer is still dependent on androgen receptor activity. Thus, new modes of inhibiting the androgen receptor may provide additional therapy options for men with prostate cancer. Two new agents have recently been developed: the CYP17 inhibitor abiraterone acetate (Johnson & Johnson, New Brunswick, N.J.), and the anti-androgen MDV3100 (Medivation, Inc., San Francisco, Calif.). Some men with castration-resistant prostate cancer respond to these agents. In men that respond, therapy resistance can also develop. Alternatively spliced androgen receptor isoforms have been identified as potentially important biomarkers of response to, or development of resistance to, therapy. That is, levels of certain alternatively spliced androgen receptor isoforms may predict response, or portend the development of resistance. Therefore, methods described herein may have utility as a diagnostic and/or therapy monitoring test for certain forms of prostate cancer.

Thus, in one aspect, the invention provides a method that can assist in identifying whether an individual is at risk of androgen depletion-independent prostate cancer. Generally, the method includes obtaining a biological sample from the individual comprising cells expressing a plurality of androgen receptor isoforms, measuring expression of at least one androgen receptor isoform, and identifying the sample as exhibiting a predetermined pattern of androgen receptor isoform expression includes, for example, wild-type isoform expression that is no more than a predetermined percentage of total androgen receptor isoform expression, isoform 112/3/2b expression that is increased compared to a normal control, or isoform 1/2/3/CE3 expression that is increased compared to a normal control. In some embodiments, the predetermined pattern of androgen isoform expression can be indicative of androgen depletion-independent (ADI) prostate cancer.

In another aspect, the invention provides an alternative method that can assist in identifying an individual at risk of androgen depletion-independent prostate cancer. Generally, this method includes evaluating whether cells in a biological sample from the subject exhibit unbalanced amplification of non-wild-type androgen receptor. Such a method includes measuring the copy number of at least a polynucleotide encoding a first non-wild-type androgen receptor and a polynucleotide encoding a second non-wild-type androgen receptor, thereby producing an expression ratio, and identifying the sample as exhibiting an expression ratio of no less than a predetermined expression ratio, thereby detecting unbalanced amplification of the androgen receptor. If the sample exhibits an expression ratio of at least a predetermined expression ratio, and thereby exhibits unbalanced amplification of androgen receptor, then the subject from whom the sample is obtained may be identified as at risk for androgen depletion-independent prostate cancer.

In either aspect, the method can further include reporting the results to a medical profession in, for example, a written, an oral, or a computer readable format.

As used herein, an individual is considered “at risk” for ADI prostate cancer if the individual exhibits androgen receptor isoform expression indicative of ADI prostate cancer regardless of whether the individual exhibits any symptoms or clinical signs of ADI prostate cancer. Thus, the method can provide diagnosis of ADI prostate cancer in advance of the individual exhibiting any symptoms of having ADI prostate cancer. Consequently, performing the method allows one to commence treatment for ADI prostate cancer earlier than if the ADI prostate cancer is detected only once the individual experiences one or more symptoms of ADI prostate cancer.

The predetermined expression ratio that is indicative of a subject at risk for ADI prostate cancer may be any expression ratio acknowledged by those of skill in the art to indicate an elevated risk of ADI. As used herein, an expression ratio is determined with respect to the least expressed non-wild-type androgen receptor and, consequently, is expressed as a x:1, wherein x represents the extent of expression of more expressed non-wild-type androgen receptor in the ratio. Thus, the predetermined expression ratio may be, for example, no less than 1.1:1, no less than 1.2:1, no less than 1.3:1, no less than 1.4:1, no less than 1.5:1, no less than 1.6:1, no less than 1.7:1, no less than 1.8:1, no less than 1.9:1, no less than 2:1, no less than 2.5:1, or no less than 3:1. In some embodiments, the predetermined expression ration may be no greater than 1,000,000:1 such as, for example, no greater than 100:1, no greater than 10: Ranges of predetermined expression ratios include all combinations of any “no less than” endpoint with any “no greater than:” endpoint.

The predetermined wild-type androgen receptor isoform expression as a percentage of total androgen receptor isoform expression that is indicative of a subject at risk for ADI can be any predetermined percentage acknowledged by those of skill in the art to indicate an elevated risk of ADI. In some embodiments, a wild-type androgen receptor isoform expression percentage of no more than 99% of total androgen receptor isoform expression may indicate that a subject is at risk for ADI such as, for example, a wild-type androgen receptor isoform expression percentage of no more than 98%, no more than 97%, no more than 96%, no more than 95%, no more than 94%, no more than 93%, no more than 92%, no more than 91%, no more than 90%, no more than 89%, no more than 88%, no more than 87%, no more than 86%, no more than 85%, no more than 75%, or no more than 50% of total androgen receptor isoform expression. In one particular embodiment, the predetermined wild-type androgen receptor isoform expression percentage that indicates a subject is at risk for ADI is no more than 95% of total androgen receptor isoform expression.

The amount of 1/2/3/2b isoform expression, compared to wild-type control that indicates that a subject is at risk for ADI can be any amount acknowledged by those of skill in the art to indicate an elevated risk of ADI. In some embodiments, such an increase in 1/2/3/2b isoform expression may be an increase so that 1/2/3/2b isoform expression reaches a level that is at least 10% of wild-type AR expression such as, for example, at least 15%, at least 20%, at least 25%, at least 26%, at least 27%, at least 28%, at least 29%, at least 30%, at least 31%, at least 32%, at least 33%, at least 34%, at least 35%, at least 40%, or at least 50% of wild-type AR expression. In one particular embodiment, the increase in 1/2/3/2b isoform expression includes an increase that results in 1/2/3/2b isoform expression reaching a level that is at least 30% of wild-type AR expression.

The amount of 1/2/3/CE3 isoform expression, compared to wild-type control that indicates that a subject is at risk for ADI can be any amount acknowledged by those of skill in the art to indicate an elevated risk of ADI. In some embodiments, such an increase in 1/2/3/CE3 isoform expression may be an increase so that 1/2/3/CE3 isoform expression reaches a level that is at least 10% of wild-type AR expression such as, for example, at least 15%, at least 20%, at least 25%, at least 26%, at least 27%, at least 28%, at least 29%, at least 30%, at least 31%, at least 32%, at least 33%, at least 34%, at least 35%, at least 40%, or at least 50% of wild-type AR expression. In one particular embodiment, the increase in 1/2/3/CE3 isoform expression includes an increase that results in 1/2/3/CE3 isoform expression reaching a level that is at least 30% of wild-type AR expression.

Because ADI prostate cancer cells acquire aberrant nuclear androgen receptor expression and activity and androgen receptor function disruption inhibits proliferation and PSA expression in ADI prostate cancer cells, it seems that the androgen receptor is able to achieve a critical level of activity in ADI prostate cancer cells, allowing their growth and survival despite androgen depletion. Thus, the androgen receptor signaling axis remains a target for therapy of ADI prostate cancer.

Various mechanisms by which prostate cancer cells are able to circumvent androgen depletion, briefly summarized below, may cooperate to elicit a critical level of androgen receptor activity in ADI prostate cancer cells.

AR Mutation

Androgen receptor mutation is a mechanism of therapy resistance operating in a subset of ADI prostate cancer. Although initial estimates suggested androgen receptor mutations occurred in 30-50% of ADI prostate cancer, more recent studies have demonstrated that the rate of androgen receptor mutation in ADI prostate cancer is much lower, at around 10% (Tilley et al., 1990 Cancer Res 50:5382-6; Thompson et al., 2003 Lab Invest 83:1709-13; Haapala et al., 2001 Lab Invest 81:1647-51; Hyytinen et al., 2002 Lab Invest 82:1591-8; Taplin et al., 1995 N Engl J Med 332:1393-8; Taplin et al., 1999 Cancer Res 59:2511-5; Taplin et al., 2003 J Clin Oncol 21:2673-8). Mechanistically, some androgen receptor mutations result in aberrant androgen receptor activation by adrenal androgens, glucocorticoids, progesterone, estradiol, or anti-androgens (Buchanan et al., 2001 Clin Cancer Res 7:1273-81; Zhao et al., 2000 Nat Med 6:703-6; Culig et al., 1993 Mol Endocrinol 7:1541-50). Other androgen receptor mutations result in constitutive, androgen independent activity, or strong activation by castrate levels of testosterone and DHT (Buchanan et al., 2001 Mol Endocrinol 15:46-56; Ceraline et al., 2004 hit J Cancer 108:152-7).

AR Overexpression and Gene Amplification

Androgen receptor overexpression has been observed in 35% of ADI tumors compared with untreated primary prostate tumors or normal prostate tissue (Ceraline et al., 2004 Int J Cancer 108:152-7). A recent gene-expression profiling study with xenograft-based models of prostate cancer progression demonstrated that the only gene significantly up-regulated during progression from androgen-dependent to ADI disease was the androgen receptor itself (Chen et al., 2004 Nat Med 10:33-9). Functionally, an increase in androgen receptor protein levels has been shown to sensitize prostate cancer cells to low levels of androgens as well as convert the androgen receptor antagonist, bicalutamide into an agonist (Chen et al., 2004 Nat Med 10:33-9). One mechanism thought to increase androgen receptor protein expression is amplification of the AR gene, which early studies reported to occur in 20-33% of ADI prostate cancer (Dehm and Tindall, 2005 Expert Rev Anticancer Ther 5:63-74). Recent FISH-based studies, which assessed AR gene amplification in circulating tumor cells from patients with ADI prostate cancer, found evidence of high-level androgen receptor amplification in 38% of samples analyzed (Leversha et al., 2009 Clin Cancer Res 15:2091-7). A similar study of circulating tumor cells reported that all patients with ADI prostate cancer (n=33) had evidence of androgen receptor amplification in at least one of their circulating tumor cells, with considerable heterogeneity between tumor cells originating from a single patient (Attard et al., 2009 Cancer Res 69:2912-8). Together, these studies demonstrate that androgen receptor amplification is a common event in ADI prostate cancer.

Ligand-Independent AR Activation

Other evidence suggests that the androgen receptor allows cells to circumvent androgen depletion by achieving a critical level of activity through ligand-independent mechanisms (Grossmann et al., 2001 J Nat Cancer Inst 93:1687-97; Rahman et al., 2004 Clin Cancer Res 10:2208-19; Feldman and Feldman, 2001 Nature Reviews Cancer 1:34-45; Heinlein and Chang, 2004 Endocr Rev 25:276-308). Ligand-independent androgen receptor activation has been shown to occur through enhanced autocrine growth factor and/or cytokine production (Grossmann et al., 2001 J Nat Cancer Inst 93:1687-97; Rahman et al., 2004 Clin Cancer Res 10:2208-19; Feldman and Feldman, 2001 Nature Reviews Cancer 1:34-45; Heinlein and Chang, 2004 Endocr Rev 25:276-308) and altered expression or activity of androgen receptor-associated co-activator proteins (Grossmann et al., 2001 J Nat Cancer Inst 93:1687-97; Rahman et al., 2004 Clin Cancer Res 10:2208-19; Feldman and Feldman, 2001 Nature Reviews Cancer 1:34-45; Heinlein and Chang, 2004 Endocr Rev 25:276-308). However, the relative contribution of ligand-independent activation to the growth and survival of ADI prostate cancer has not been established in vivo. This may be because testosterone levels in recurrent prostate tumors can persist at a level high enough to weakly activate the androgen receptor (Mohler et al., 2004 Clin Cancer Res 10:440-8). In early-phase clinical trials, second generation non-steroidal anti-androgens such as RD162 and MDV3100 (Tran et al., 2009 Science 324:787-90), and agents that promote a “super-castrated” state such the CYP17 inhibitor abiraterone acetate (Attard et al., 2008 J Clin Oncol 26:4563-71), induced initial serum PSA declines of at least 50% in roughly one-half of patients with ADI prostate cancer. However, the remaining one-half of patients with ADI prostate cancer did not respond to these experimental therapies, supporting the possibility that ligand-independent androgen receptor activation could be important for therapy resistance of ADI prostate cancer in vivo.

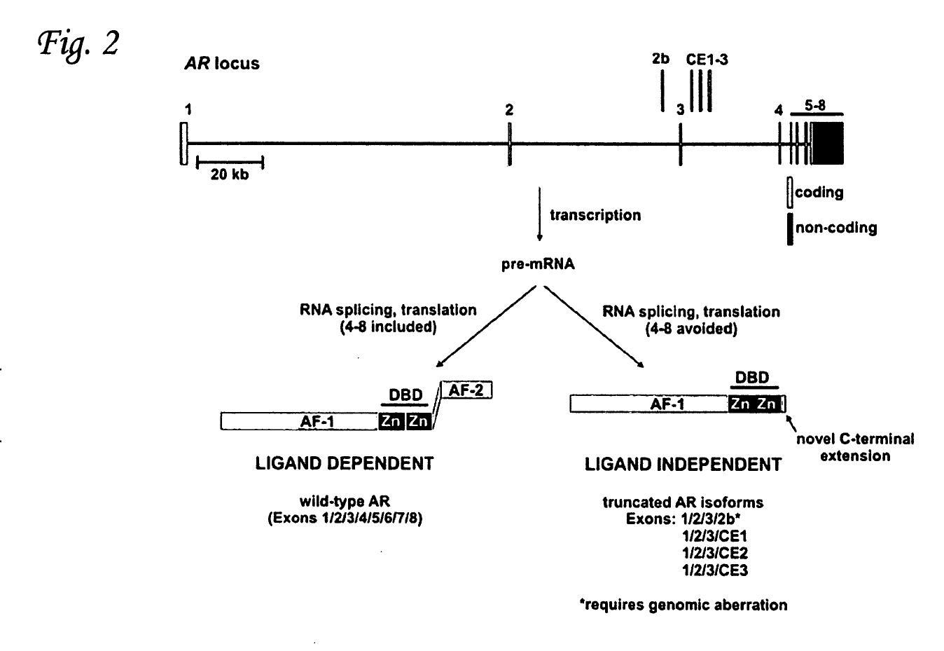

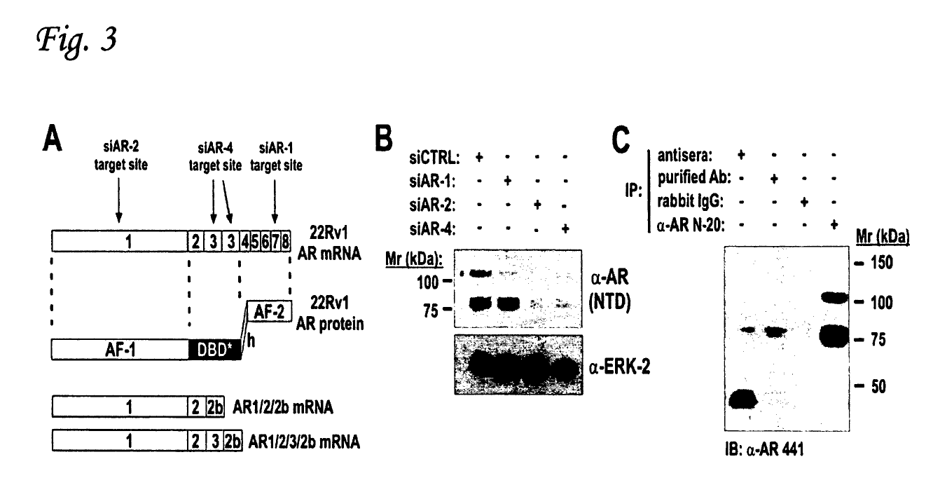

We have investigated mechanisms of prostate cancer therapy resistance in cell- and xenograft-based models of prostate cancer progression with a focus on ligand-independent androgen receptor activation (Dehm and Tindall, 2006 J Biol Chem 281:27882-93; Dehm and Tindall, 2007 Mol Endocrinol 21:2855-63). One model of interest is the ADI 22Rv1 prostate cancer cell line, which was derived from an androgen-dependent CWR22 prostate cancer xenograft that relapsed during androgen depletion (Sramkoski et al., 1999 In Vitro Cell Dev Biol Anim 35:403-9). We have observed that 22Rv1 cells displayed differential sensitivity to siRNAs targeted to AR Exon 1 versus AR Exon 7 (Dehm et al., 2008 Cancer Res 68:5469-77; and FIGS. 2, 3A, and 3B). The Exon 1 and Exon 7 targeted siRNAs were purchased from Dharmacon, Inc. (Lafayette, Colo.). The sequences are as follows:

Exon 1-targeted:siGENOME duplex (2) CA# D-003400-02, AR

Sense:
(SEQ ID NO: 1)
5′-CAAGGGAGGUUACACCAAAUU
Antisense:
(SEQ ID NO: 2)
5′-UUUGGUGUAACCUCCCUUGUU

Exon 7-targeted:siGENOME duplex (1) CA# D-003400-01, AR

Sense
(SEQ ID NO: 3)
5′-GGAACUCGAUCGUAUCAUUUU
Antisense:
(SEQ ID NO: 4)
5′-AAUGAUACGAUCGAGUUCCUU

The siRNA targeted to AR Exon 1, but not AR Exon 7, knocked-down the expression of a ˜80 kDa androgen receptor protein species in 22Rv1 cells, which was previously thought to be a proteolytic product of full-length androgen receptor (Tepper et al., 2002 Cancer Res 62:6606-14; Libertini et al., 2007 Cancer Res 67:9001-5). Importantly, AR Exon 1-targeted siRNAs, but not AR Exon 7-targeted siRNAs, inhibited androgen-independent proliferation of 22Rv1 cells and androgen-independent expression of androgen receptor target genes (Dehm et al., 2008 Cancer Res 68:5469-77). Subsequent 3′ RACE experiments led to our discovery of AR Exon 2b is a novel exon in the androgen receptor locus that has an in-frame stop codon, which can be spliced after either AR Exon 2 or AR Exon 3 and give rise to truncated androgen receptor species that lack the LBD/AF-2 module (FIG. 2). The only way AR Exon 2b could be spliced after AR Exon 3 is as a result of a chromosomal aberration that alters the genomic orientation of these exons, which is the case in the 22Rv1 cell line. In our initial characterization of these novel androgen receptor isoforms, which we termed AR 1/2/2b and AR 1/2/312b, we observed constitutive, ligand-independent activity. Moreover, by employing siRNAs specific for AR Exon 2b, we showed that specific targeting of this novel androgen receptor exon led to selective knock-down of truncated androgen receptor isoforms in 22Rv1 cells, which inhibited androgen-independent, but not androgen-dependent, cell proliferation and expression of androgen receptor target genes (Dehm et al., 2008 Cancer Res 68:5469-77). These data demonstrated that truncating the androgen receptor through alternative splicing of a novel androgen receptor exon was a mechanism of ligand-independent androgen receptor activation that could mediate the ADI phenotype of prostate cancer cells.

In addition to AR Exon 2b, three more AR Exons, termed Cryptic Exons 1-3 (CE1-3), are tightly grouped downstream of AR Exon 3 (FIG. 2). Similar to AR Exon 2b, Exons CE1-3 can be spliced after AR Exon 3, and by virtue of containing premature translation stop codons, give rise to truncated androgen receptor isoforms lacking the LBD/AF-2 module (Guo et al., 2009 Cancer Res 69:2305-13; Hu et al., 2009 Cancer Res 69:16-22; and FIG. 2). As used herein, the Cryptic Exon isoforms are termed AR 1/2/3/CE1, AR 1/2/3/CE2, and AR 1/2/3/CE3 (FIG. 2).

AR 1/2/3/CE3 mRNA is increased in ADI prostate cancer compared to hormone naive prostate cancer, and is predictive of biochemical recurrence following surgery (Hu et al., 2009 Cancer Res 69:16-22). Similar to our findings with AR 1/2/3/2b, AR 1/2/3/CE3 is constitutively active and may induce ADI growth of androgen-dependent LNCaP cells in vitro and in vivo (Guo et al., 2009 Cancer Res 69:2305-13). Polyclonal antibodies that specifically recognized the AR 1/2/3/CE3 isoform have been generated. Moreover, the AR 1/2/3/CE3 isoform is expressed in 22Rv1 cells (Guo et al., 2009 Cancer Res 69:2305-13; Hu et al., 2009 Cancer Res 69:16-22), although immunodepletion studies demonstrated that AR 1/2/3/CE3 was not the predominant truncated androgen receptor isoform in these cells (Hu et al., 2009 Cancer Res 69:16-22). The AR 1/2/3/CE3-specific polyclonal antibodies recognized AR 1/2/3/CE3 in clinical samples of ADI prostate cancer (Guo et al., 2009 Cancer Res 69:2305-13). Surprisingly, AR 1/2/3/CE3 protein expression has been reported in benign prostate tissue and hormone naive prostate cancer.

AR 1/2/3/2b Protein is Expressed in 22Rv1 Cells

Truncated androgen receptor from 22Rv1 cells appears as a doublet in Western blot experiments (FIG. 3B). Because 3′ RACE experiments identified AR 1/2/2b and AR 1/2/3/2b as alternatively spliced androgen receptor isoforms in this cell line, we pursued the hypothesis that these two discrete bands represented AR 1/2/2b and AR 1/2/3/2b (Dehm et al., 2008 Cancer Res 68:5469-77). However, based on the reports by other groups that other truncated androgen receptor isoforms existed (Guo et al., 2009 Cancer Res 69:2305-13; Hu et al., 2009 Cancer Res 69:16-22), and that the AR 1/2/2b isoform did not display transcriptional activity in their assay systems (Guo et al., 2009 Cancer Res 69:2305-13), we pursued more stringent tests to differentiate between AR 1/2/2b and AR 1/2/3/2b in the 22Rv1 cell line. To this end, we tested siRNAs targeted to AR Exon 1, AR Exon 3, or AR Exon 7 (FIG. 3A). As expected, AR Exon 7-targeted siRNA selectively knocked down expression of full-length androgen receptor (FIG. 3B). However, both AR Exon 1-targeted siRNAs and AR Exon 3-targeted siRNAs knocked down expression of all androgen receptor isoforms (FIG. 3B). This suggests that the AR 1/2/2b isoform is not expressed in these cells, because mRNA containing Exons 1/2/2b would be resistant to Exon 3-targeted siRNA.

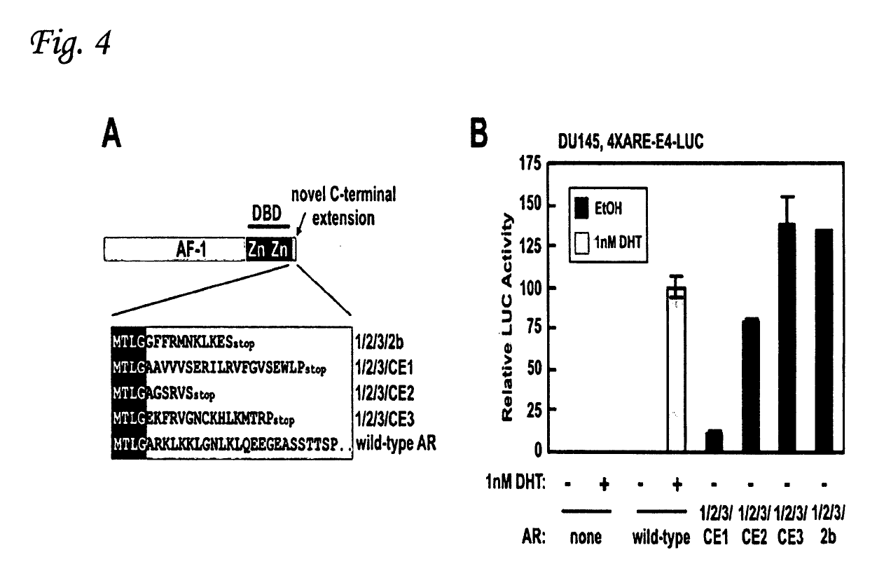

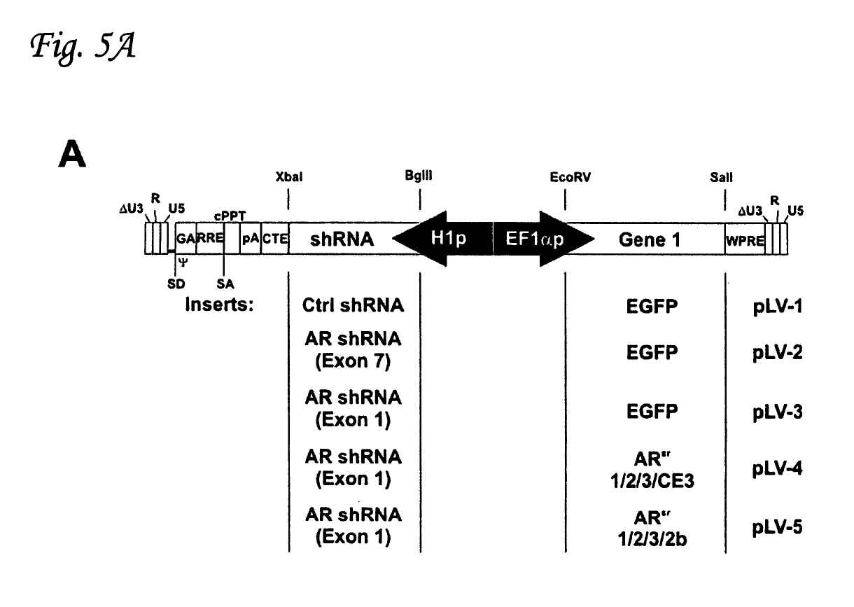

To test whether AR 1/2/2b and/or AR 1/2/3/2b protein is expressed in 22Rv1 cells, we generated a polyclonal antibody specific for these isoforms by immunizing rabbits with a peptide representing the novel C-terminal extension encoded by AR Exon 2b (FIGS. 3C and 4A). AR Exon 2 and AR Exon 3 have the same reading-frame, so the AR Exon 2b-derived sequence is the same for both AR 1/2/2b and AR 1/2/3/2b (Dehm et al., 2008 Cancer Res 68:5469-77). In immunoprecipitation experiments with 22Rv1 lysates, we observed that both crude antisera and antibody purified on an immobilized AR Exon 2b peptide column immunoprecipitated a species that was recognized by a monoclonal antibody specific for the androgen receptor NTD (FIG. 3C). This species migrated with the same mobility as the largest of the 22Rv1 truncated species at roughly ˜80 kDa, which matches the predicted molecular weight of AR 1/2/3/2b, but not AR 1/2/2b (FIG. 3C).

In summary, we found that AR Exon 3-targeted siRNA knocked down the expression of all truncated androgen receptor isoforms in 22Rv1 cells. Furthermore, an antibody raised against AR Exon 2b-derived peptides immunoprecipitated a single androgen receptor isoform of ˜80 kDa, which matches the predicted molecular weight of the AR 1/2/3/2b protein. This suggests that AR 1/2/3/2b, but not AR 1/2/2b, is the predominant androgen receptor protein isoform derived from AR Exon 2b splicing in 22Rv1 cells.

Exon 3-Truncated AR Isoforms have Differential Transcriptional Activities

To compare AR 1/2/3/2b activity to other truncated androgen receptor isoforms and determine whether there are any differences in function that could arise from differences in the C-terminal (FIG. 4A), we generated expression vectors for AR 1/2/3/CE1, AR 1/2/3/CE2, AR 1/2/3/CE3, and AR 1/2/3/2b, and tested their transcriptional activities in AR-null DU145 cells (FIG. 4B). All isoforms displayed constitutive, ligand-independent androgen receptor activity; however, the activities of AR 1/2/3/CE3 and AR 1/2/3/2b were highest. AR 1/2/3/CE1 displayed the lowest level of transcriptional activity in this assay (FIG. 4B). These data suggest that alternative androgen receptor exons not only encode premature translation stop codons, but that they may also encode C-terminal regulatory elements.

### AR 1/2/3/2b and AR 1/2/3/CE3 Can Rescue Growth Inhibition Caused by Knock-Down of Truncated AR

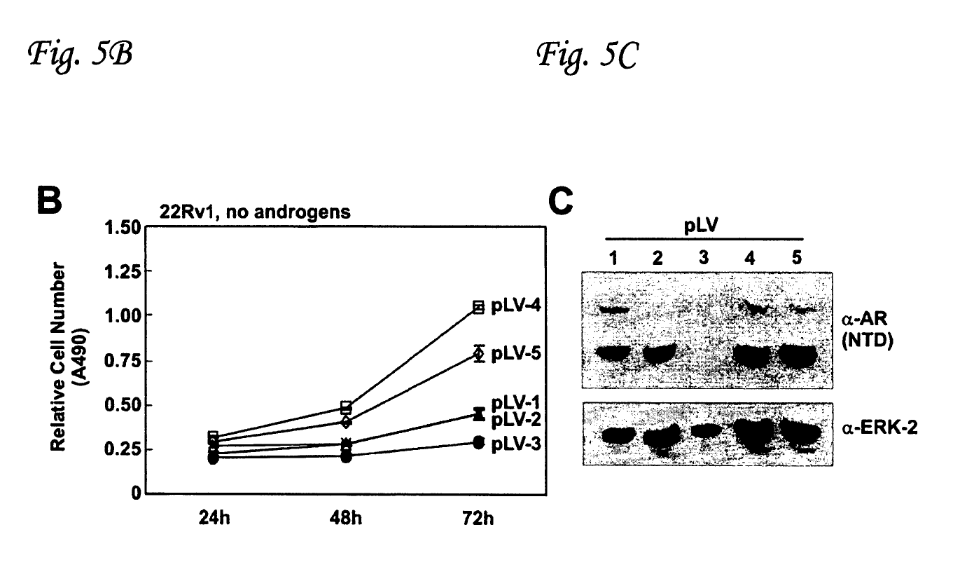

Our reporter assay data conflicts with data from another study, which reported that AR 1/2/3/CE3 was the only truncated androgen receptor isoform with constitutively strong transcriptional activity (Guo et al., 2009 Cancer Res 69:2305-13). This may be the result of differences in experimental set-up, such as cell line, promoter reporter construct, or protein expression level. To address whether AR 1/2/3/CE3 is the only truncated androgen receptor isoform that can promote ADI growth of prostate cancer cells, we developed a bi-directional lentivirus expression system that can accept various shRNA/gene combinations (FIG. 5A) and performed knock-down/rescue experiments in 22Rv1 cells (FIG. 5). In all the prostate cancer cell lines we have tested, transduction efficiency is consistently >98%, and remains at this level over months of in vitro culture. ADI growth of 22Rv1 cells was inhibited by shRNA targeted to AR Exon 1, but not AR Exon 7 (FIG. 5B). Exon 1-targeted shRNA were synthesized, annealed, phosphorylated, and inserted upstream of a histone H1 promoter. Exon 7-targeted shRNA were synthesized, annealed, phosphorylated, and inserted upstream of a histone H1 promoter. The shRNA sequences are shown in Table 1.

TABLE 1

| | | SEQ ID NO |
|---|---|---|
| EXON 1 FWD | gatccccGCCATACTGCATGGCAGCAttcaagagaTGCTGCCA | SEQ ID NO: 5 |
| | TGCAGTATGGCtttttggaaa | |
| EXON 1 REV | agcttttccaaaaaGCCATACTGCATGGCAGCAtctcttgaaTGCTGCCAT | SEQ ID NO: 6 |
| | GCAGTATGGCggg | |
| EXON 7 FWD | gatccccGGAACTCGATCGTATCATTttcaagagaAATGATACGATC | SEQ ID NO: 7 |
| | GAGTTCCtttttggaaa | |
| EXON 7 REV | agcttaccaaaaaGGAACTCGATCGTATCATTtctcttgaaAATGA | SEQ ID NO: 8 |
| | TACGATCGAGTTCCggg | |

Re-expression of shRNA-resistant versions of AR 1/2/3/CE3 and AR 1/2/3/2b both restored, and even enhanced, ADI growth of these cells (FIG. 5B). Interestingly, we observed a slight restoration of full-length androgen receptor expression caused by expression of these truncated androgen receptor isoforms (FIG. 5C). We suspect this is because the endogenous androgen receptor gene is androgen-regulated and the strong activity of the truncated androgen receptor isoforms enhanced endogenous androgen receptor gene expression to a level that challenged the ability of AR-targeted shRNA to maintain efficient knock-down. Nevertheless, these data demonstrate that each of AR 1/2/3/CE3 and AR 112/3/2b can independently promote ADI growth of 22Rv1 cells.

### Unbalanced AR Amplification in ADI Prostate Cancer Cells and Synthesis of Truncated AR Isoforms

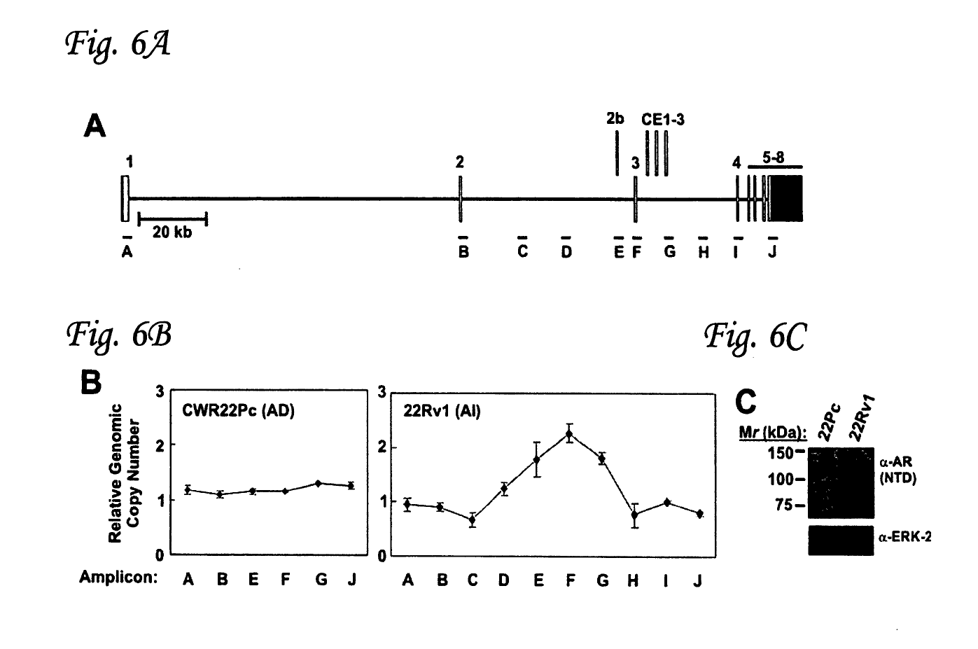

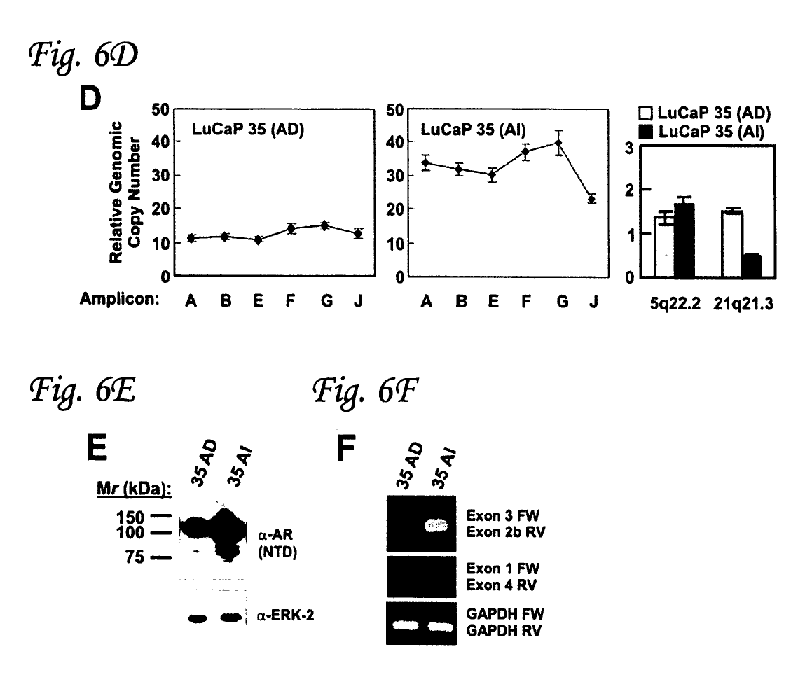

Androgen receptor mRNA in 22Rv1 cells contains a duplication of AR Exon 3, which results in a larger androgen receptor protein consisting of three zinc fingers in its DBD (FIG. 3A). This AR Exon 3-duplicated androgen receptor does not exist in androgen-dependent CWR22Pc cells, which were derived from the parental CWR22 xenograft (FIG. 6B, FIG. 6C), and has not been observed in any other prostate cancer tumors or cell lines (Tepper et al., 2002 Cancer Res 62:6606-14). To investigate the relationship between AR Exon 3-duplicated androgen receptor and the ability to synthesize high levels of truncated androgen receptor proteins, we studied genomic duplication within the AR locus by quantitative real-time genomic PCR. Strikingly, in 22Rv1 cells, we observed focal and selective amplification of a small island in the AR locus encompassing Exons 2b, Exon 3, CE1, CE2, and CE3 (FIG. 6B). This amplification was not observed in the androgen-dependent CWR22Pc cell line (FIG. 6B). By Western blot, we confirmed that this genomic aberration corresponded with high-level synthesis of a larger androgen receptor protein (AR Exon 3-duplicated), and the high-level expression of truncated androgen receptor transcripts in 22Rv1 cells (FIG. 6C). The LuCaP 35 xenograft model of prostate cancer progression expresses a high level of truncated androgen receptor mRNAs and proteins (Dehm et al., 2008 Cancer Res 68:5469-77; Hu et-al., 2009 Cancer Res 69:16-22). We therefore interrogated the AR locus in androgen-dependent (AD) and ADI versions of LuCaP 35 xenografts (FIG. 6D). AD LuCaP 35 xenografts displayed balanced amplification of the AR locus, with a total of 10-12 gene copies. Interestingly, AI LuCaP 35 xenografts, which are relapsed, ADI versions of the parental LuCaP 35 tumor (Corey et al., 2003 Prostate 55:239-46), displayed more pronounced AR amplification, but in an unbalanced fashion. We observed between 30-40 copies of a region spanning Exons 1-CE3, but only 20-23 copies of a region containing AR Exon 8 (FIG. 6D). This unbalanced amplification corresponded with increased androgen receptor protein expression, as well as high-level expression of truncated androgen receptor in AI LuCaP 35 cells (FIG. 6E). Remarkably, via RT-PCR, we also determined that unbalanced amplification corresponded with expression of AR 1/2/3/2b mRNA (FIG. 6F). This indicates that at least one of the >30 copies of the AR locus in AI LuCaP 35 xenografts has the androgen receptor exon configuration required for splicing of AR Exon 2b downstream from AR Exon 3. These data also show that two independent models of ADI prostate cancer have unbalanced AR amplification, with a common breakpoint between AR Exons CE3 and 8 (FIG. 6A).

Thus, in summary, truncated androgen receptor protein isoforms expressed in 22Rv1 cells contain the entire androgen receptor NTD and DBD, which are encoded by Exon 1, Exon 2, and Exon 3, and unique C-terminal ends, which are encoded by novel 3′ exons. AR 1/2/3/2b protein is expressed in 22Rv1 cells, is constitutively active, and can independently support the ADI growth of 22Rv1 prostate cancer cells. Androgen receptor isoforms truncated by splicing of AR Exon 2b, CE1, CE2, or CE3 are all constitutively active, but have varying strengths of transcriptional activity. ADI cells in both the CWR22 and LuCaP 35 models of prostate cancer progression display unbalanced androgen receptor amplification, which correlates with enhanced expression of truncated androgen receptor protein isoforms and the ability to synthesize AR 1/2/3/2b mRNAs. Finally, a common genomic breakpoint exists between AR Exons CE3 and 8 in two models of ADI prostate cancer. Based on these data, unbalanced AR amplification can yield aberrant AR loci, which permits efficient synthesis of truncated, transcriptionally active androgen receptor isoforms that my be involved in mediating the ADI phenotype of lethal prostate cancer.

One can, therefore, examine either unbalanced AR amplification or expression of truncated androgen receptor isoforms to identify individuals at risk for ADI prostate cancer. Identifying a subject as at risk for ADI prostate cancer can provide guidance to initiate or modify treatment of the prostate cancer. For example, in circumstances in which an individual is identified as at risk for ADI prostate cancer prior to any treatment being initiated, treatment regimens that target the androgen receptor may be avoided as less likely to provide effective treatment. In other circumstances, a subject receiving treatment for prostate cancer may be identified as at risk for ADI prostate cancer, indicating that tumor cells may be adapting to become resistant to current therapy. In such circumstances, identifying the subject as at risk for ADI prostate cancer may indicate that modifying the existing therapy is appropriate. In some circumstances, the modification can include administering to the subject a pharmaceutical composition that is effective for treating ADI prostate cancer.

Also, one can further analyze unbalanced AR amplification and expression of truncated androgen receptor isoforms in ADI prostate cancer. To do so, one can determine the locations of AR genomic breakpoints in 22Rv1 cells and AI LuCaP 35 xenografts. Targeted genomic copy number analysis of the AR locus in 22Rv1 cells and LuCaP 35 xenografts indicates that a common genomic breakpoint exists between Exons CE3 and 8 (FIG. 6). If this is a recurrent genetic aberration in ADI prostate cancer, it may provide important prognostic and/or diagnostic information. This is particularly relevant to emerging clinical trials with second generation anti-androgens and CYP17 inhibitors (Attard et al., 2009 Cancer Res 69:2912-8; Tran et al., 2009 Science 324:787-90), where the presence of such breakpoints in circulating tumor cells might serve as a mechanism-based surrogate marker for resistance to these agents.

Copy number analysis in 22Rv1 cells suggests a breakpoint in the AR locus occurs between amplicons G and H (FIGS. 6A and B), which are separated by approximately 15 kb. To narrow down the breakpoint region, one can design PCR primer pairs that will amplify 80 bp-150 bp products every 1 kb between amplicons G and H. One can then use the PCR primers in quantitative PCR reactions with CWR22Pc and 22Rv1 genomic DNA, and calculate copy number by extrapolating against a standard curve generated from normal prostate epithelial cell genomic DNA. This will allow determination of the genomic breakpoint to 1 kb resolution. One can then design nested primers on the duplicated side of the breakpoint and use ligation mediated PCR (LM-PCR; Mueller et al., “Ligation-mediated PCR for genomic sequencing and footprinting,” in Current Protocols in Molecular Biology 2001; Chapter 15: Unit 15.3) to identify genomic DNA sequence adjacent to the duplication. PCR products can be cloned and sequenced, and novel sequences can be mapped to the genome. With AI LuCaP 35 xenografts, one can take the exact same approach, but first one can assess genomic copy number for amplicons H and I (FIG. 6A) to map more precisely this breakpoint and guide decisions regarding the region that is analyze at 1 kb resolution.

The method may yield nucleotide-level information on the breakpoint between amplicons G and J in these models of ADI prostate cancer. In the case of 22Rv1 cells, our preliminary data shows that the 5′ end of the focal DNA duplication in 22Rv1 cells is located between amplicons D and E (FIG. 6B). Therefore, we expect that sequences identified by LM-PCR in 22Rv1 cells will map to this region.

Further, one can determine whether unbalanced AR amplification occurs in ADI versus AD prostate cancer. Our studies with two independent cell-based models of prostate cancer progression suggest that unbalanced AR amplification may be a mechanism that promotes efficient synthesis of truncated androgen receptor proteins in ADI prostate cancer. FISH and CGH-based studies have failed to identify breakpoints within the AR locus. Possible explanations for these failures may include, for example, the size of AR FISH probes (>150 kb) and the inability of CGH to accurately quantify genomic copy number. In contrast, a targeted and quantitative approach may be preferred to assess whether unbalanced AR amplification occurs in human prostate cancer.

Through the University of Minnesota Masonic Cancer Center Tissue Procurement Core Facility, we have access to 103 specimens of prostate cancer at various stages, which contain >50% neoplasia as determined by pathological examination of adjacent tissue slides. Samples of normal prostate tissue may be used as controls. All tissues were processed by flash-freezing in liquid nitrogen.

To quantitatively measure AR gene copy number along the length of the AR gene, one can isolate genomic DNA from prostate cancer samples and perform real-time genomic PCR as shown in FIG. 6. For initial screening of all tissues, one can use primer sets that interrogate amplicons A (Exon 1), B (Exon 2), F (Exon 3), and J (Exon 8) (FIG. 6). In some cases, an F:J copy number ratio>1.5 signifies unbalanced AR amplification. For those samples where unbalanced AR amplification is observed, one can investigate these samples in more detail and map the precise location of their breakpoints exactly as described above. One can also employ appropriate control primers to amplify, for example, 5q22.2 and 21q21.3 to verify AR-specificity of amplification events as shown in FIG. 6D.

In some cases, unbalanced AR amplification can be an adaptive response to androgen depletion (FIGS. 6B and 6D). Therefore, in some cases, one can observe an elevated F:J amplicon ratio in a subset of ADI tumor and xenograft samples, but not androgen-dependent prostate cancer or normal prostate tissue.

One can also measure absolute levels of androgen receptor mRNA isoforms in prostate cancer cell lines, xenografts, and clinical tissues. AR 1/2/3/CE3 mRNA and protein expression has been observed in benign prostate tissue as well as primary androgen-dependent prostate cancer (Guo et al., 2009 Cancer Res 69:2305-13; Hu et al., 2009 Cancer Res 69:16-22), suggesting that in at least some circumstances simple expression of this isoform does not predict responsiveness to androgen depletion. Because androgen receptor proteins dimerize at promoters and enhancers of androgen receptor-responsive target genes, expression of wild-type androgen receptor or less-active truncated androgen receptor proteins such as AR 1/2/3/CE1 (FIG. 4) could offset the strong activity of AR. 1/2/3/CE3 in normal prostate tissue or androgen-dependent prostate cancer. Unbalanced AR amplification in ADI prostate cancer may disrupt the normal ratios of androgen receptor isoform expression, thereby leading to unchecked ligand-independent activity of highly-active truncated androgen receptor isofolins. Therefore, the extent to which unbalanced AR amplification correlates with altered expression patterns of androgen receptor isoforms can provide diagnostic and/or prognostic information.

Previous studies attempted to measure relative expression of androgen receptor mRNA isoforms in prostate cancer cell lines and tissues (Guo et al., 2009 Cancer Res 69:2305-13 Hu et al., 2009 Cancer Res 69:16-22). These studies inappropriately employed the 2^{-ΔΔCt} calculation method, which should only be used for comparing the expression levels of one gene (relative to an internal control) among different samples. This method is inappropriate for quantifying relative expression levels of multiple genes within the same sample because different primer sets may not amplify their targets with the exact same efficiency. Data generated this way can lead to results that are inaccurate by an order of magnitude or more.

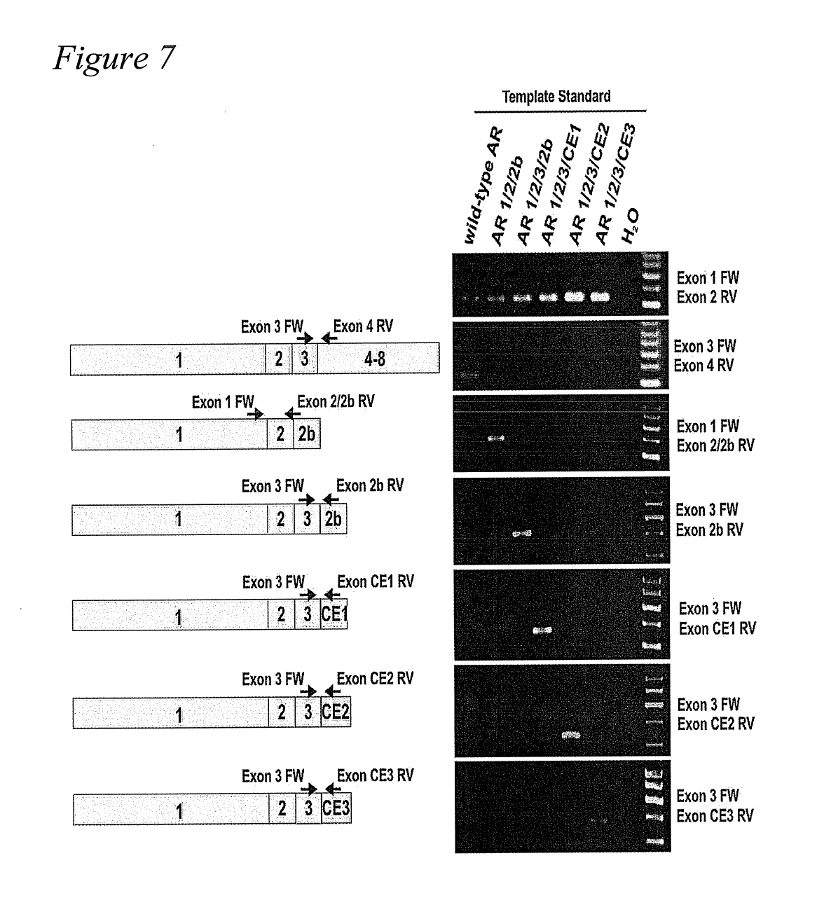

Instead, one can employ quantitative RT-PCR to determine absolute copy number for all known major androgen receptor mRNA species: wild-type androgen receptor (Exons 1-8), AR 1/2/2b, AR 1/2/3/2b, AR 1/2/3/CE1, AR 1/2/3/CE2, AR 1/2/3/CE3 (FIG. 7). One can also include a primer set that will amplify all androgen receptor mRNAs with Exon 1 spliced to Exon 2, which will provide an assessment of the total abundance of androgen receptor mRNAs irrespective of the constitution of their 3′ exons. We have designed the requisite primer pairs that can discriminate between these species (FIG. 7). Discrimination between AR 1/2/2b and AR 1/2/3/2b can be achieved by using a reverse primer that hybridizes to the Exon 2/2b splice junction of AR 1/2/2b mRNA (FIG. 7). Template standards for these isoforms may be used to generate standard curves for calculation of absolute copy number (FIG. 7). Templates are shown in Table 2. Primer sequences are shown in Table 3.

TABLE 2

| Template (plasmid) | FWD primer | REV primer |
|---|---|---|
| 1/2/3/CE1 | qSPLICE 1/2/3/2b F2 | qRT CE1 RV |
| 1/2/3/CE2 | qSPLICE 1/2/3/2b F2 | qRT CE2 RV |
| 1/2/3/CE3 | qSPLICE 1/2/3/2b F2 | qRT CE3 RV |
| 1/2/3/2b | qSPLICE 1/2/3/2b F2 | qRT Ex2b REV Corr |
| 1/2/2b | qSPLICE 1/2/2b F1 | qSPLICE 1/2/2b R |
| Full-length WT | qSPLICE 1/2/3/2b F2 | qRT Ex4 RV |
| Full-length WT | qRT Exon1 FW | qRT Exon2 RV |

TABLE 3

| Primer | Nucleotide sequence | SEQ ID NO |
|---|---|---|
| qSPLICE 1/2/3/2b F2 | 5′-AAC AGA AGT ACC TGT GCG CC-3′ | SEQ ID NO: 9 |
| qRT CE1 RV | 5′-TGA GAC TCC AAA CAC CCT CA-3′ | SEQ ID NO: 10 |
| qRT CE2 RV | 5′-TAT GAC ACT CTG CTG CCT GC-3′ | SEQ ID NO: 11 |
| qRT CE3 RV | 5′-TCA GGG TCT GGT CAT TTT GA-3′ | SEQ ID NO: 12 |
| qRT Exon2b REV | 5′-TTC TGT CAG TCC CAT TGG TG-3′ | SEQ ID NO: 13 |
| Corr | | |
| qSPLICE 1/2/2b F1 | 5′-TGG ATG GAT AGC TAC TCC GG-3′ | SEQ ID NO: 14 |
| qSPLICE 1/2/2b R | 5′-GTT CAT TCT GAA AAA TCC TTC AGC-3′ | SEQ ID NO: 15 |
| qRT Exon1 FW | 5′-TGG ATG GAT AGC TAC TCC GG-3′ | SEQ ID NO: 16 |
| qRT Exon2 RV | 5′-CCC AGA AGC TTC ATC TCC AC-3′ | SEQ ID NO: 17 |
| qRT EX4 RV | 5′-TTC AGA TTA CCA AGT TTC TTC AGC-3′ | SEQ ID NO: 18 |

For human tissue analysis, one can isolate RNA and perform reverse transcription using oligo(dT) priming followed by quantitative real-time PCR. ADI tissue samples, androgen-dependent prostate cancer tissue samples, and normal prostate tissue samples can be analyzed. One-way ANOVA tests of the PCR data can determine whether: (1) the ratio of truncated androgen receptor mRNAs to full-length androgen receptor mRNA changes during prostate cancer progression, (2) the expression levels of individual truncated androgen receptor mRNAs change during prostate cancer progression, (3) the ratio of AR 1/2/3/2b mRNA to total androgen receptor mRNA changes during prostate cancer progression, and (4) the ratio of AR 1/2/3/CE3 mRNA to total androgen receptor mRNA changes during prostate cancer progression.

One can perform the similar analyses using paired AD/AI xenograft models as well as paired AD/AI CWR22Pc/22Rv1 cells. For the CWR22Pc/22Rv1 experiments, one can also quantify androgen receptor isoform expression in cells pre-treated for 30 minutes with actinomycin D, which inhibits transcription and will allow time for degradation of unstable RNAs. Data may be subjected to standard statistical tests such as, for example, paired t-test for comparison of two sample sets or one-way ANOVA test for comparison of more than two sample sets.

In some cases, the level of androgen receptor mRNA determined using Exon 1/2 PCR primer pairs may indicate the total amount of AR mRNA being expressed. The combined levels of wild-type AR, AR 1/2/2b, AR 1/2/3/2b, AR 1/2/3/CE1, AR 1/2/3/CE2, and AR 1/2/3/CE3 may represent >95% of this expression total, with wild-type androgen receptor representing the predominant isoform in normal prostate tissue and androgen-dependent prostate cancer. Truncated androgen receptor isoforms may represent a much larger fraction of the total androgen receptor mRNA expression in ADI prostate cancer, particularly in samples with unbalanced AR amplification.

In some cases, mRNA expression might not correlate with protein expression, as we have demonstrated for AR 1/2/2b (FIG. 3). However, actinomycin D experiments in 22Rv1 cells will differentiate between mRNAs that are stable and those that are rapidly eliminated through mechanisms such as nonsense-mediated RNA decay. Therefore, the actinomycin D experiment may provide insight into any disconnect between mRNA and protein expression. As designed, these experiments may identify whether aberrations in the AR locus lead to altered abundance of androgen receptor mRNAs.

Further, one can determine the biochemical properties of individual truncated androgen receptor isoforms. To do so, one can determine whether individual truncated androgen receptor isoforms can support the ADI growth of prostate cancer cells. Individual truncated androgen receptor isoforms have differing strengths of transcriptional activity. There may also be cell-type specific as well as promoter-specific differences in transcriptional activity of these isoforms (Dehm et al., 2008 Cancer Res 68:5469-77; Guo et al., 2009 Cancer Res 69:2305-13; Hu et al., 2009 Cancer Res 69:16-22). One can systematically test whether individual truncated isoforms have the capacity to support ADI growth of prostate cancer cells.

Bidirectional lentiviral vectors with the AR 1/2/3/2b and AR 1/2/3/CE3 isoforms are capable of rescuing the inhibition of ADI growth of 22Rv1 cells caused by shRNA directed to AR Exon 1 (FIG. 5). In some embodiments, the bidirectional lentiviral vector can be based on the vector described in Amendola et al. (2005), Nature Biotechnology 23:108-116. To create the constructs shown in FIG. 5, we removed the CMV/LNGFR cassette of the Amendola et al. lentiviral vector, and replaced it with the appropriate histone H1 promoter/shRNA cassette (Table 3). We then replaced the GFP cassette in these constructs with the appropriate shRNA-resistant isoforms of the androgen receptor. The shRNA-resistant isoforms of the androgen receptor were constructed as described in EXAMPLE 1, below.

One can construct similar bidirectional lentiviral vectors containing cDNAs encoding shRNA-resistant versions (denoted AR^{sr}) of AR 1/2/3/CE1, AR 1/2/3/CE2, and wild-type AR as described in EXAMPLE 1. Using the 293T cell line, one can produce viruses for LV1-5 (FIG. 5) as well as newly-constructed lentiviral vectors containing AR^{sr}, AR^{sr} 1/2/3/CE1, and AR^{sr} 1/2/3/CE2. One can infect 22Rv1 cells with a range of viral titers obtained from 293T viral supernatant and test androgen receptor expression for each lentivirus-infected sample via Western blot with antibodies targeted to the androgen receptor NTD. Cells expressing levels of shRNA-resistant androgen receptor isoforms that are comparable to endogenous levels of androgen receptor proteins can be used in subsequent experiments.

One can assess the effect of androgen receptor knock-down and isoform re-expression on the in vitro growth of 22Rv1 cells in medium containing androgen-depleted serum. To do so, equal numbers of cells can be seeded and cell numbers can be measured every 48 hours over a 2-week time course by crystal violet staining (FIG. 5). Knock-down of truncated androgen receptor isoforms inhibits cell proliferation, but has no effect on apoptosis (Dehm et al., 2008 Cancer Res 68:5469-77; Guo et al., 2009 Cancer Res 69:2305-13). Therefore, one can test the effect of androgen receptor isoform re-expression on cell proliferation using a BrdU incorporation assay (Roche Diagnostics, Corp.; Indianapolis, Ind.) as previously described (Dehm et al., 2008 Cancer Res 68:5469-77). To test for any effects at the level of apoptosis, one can assess PARP cleavage and Annexin V/propidium iodide staining as previously described (Dehm et al., 2008 Cancer Res 68:5469-77). One can also isolate RNA from cells and interrogate androgen receptor function by monitoring expression of the androgen-induced PSA, hK2, SCAP, and TMPRSS2 (Lin et al., 1999 Cancer Res 59:4180-4) genes, as well as the androgen-repressed maspin tumor suppressor gene (Zhang et al., 1997 Proc Natl Acad Sci USA 94:5673-8) by quantitative real-time RT-PCR.

To complement these in vitro studies, one can also assess the abilities of lentivirus-infected cells to grow as orthotopic xenografts in immunocompromised mice. One can castrate athymic (Foxn1nu) mice 2 weeks prior to tumor cell implantation, which will allow serum androgens to nadir. At this time, the anterior prostate of castrated mice may be exposed by pushing the bladder and seminal vesicles through a transverse abdominal incision. One can use a 30-gauge needle to inject approximately 10⁶ cells into the anterior prostates of, for example, 10 mice per cell line. These animal numbers can yield the requisite statistical power, as demonstrated by previous studies with similar models (Sramkoski et al., 1999 In Vitro Cell Dev Biol Anim 35:403-9; Guo et al., 2009 Cancer Res 69:2305-13; Corey et al., 2003 Prostate 56:110-4). Abdominal incisions can then be closed with vicryl 3-0 sutures. Tumor establishment and growth can be monitored by palpation as described (Corey et al., 2003 Prostate 56:110-4) as well as bi-weekly cheek bleeds and ELISA-based total serum PSA measurement (Beckman Coulter, Inc.; Brea, Calif.). Animals can be sacrificed when tumors from the pLV-1 group (control shRNA/EGFP) are established (within 4-6 weeks; Sramkoski et al., 1999 In Vitro Cell Dev Biol Anim 35:403-9; Guo et al., 2009 Cancer Res 69:2305-13; Corey et al., 2003 Prostate 56:110-4). Upon sacrifice, tumors can be removed, weighed, measured, and frozen. Androgen receptor expression can be assessed by Western blot, and androgen receptor function can be tested by measuring expression of target genes by quantitative RT-PCR.

In some cases, androgen receptor shRNA directed to Exon 1 can inhibit in vitro proliferation and tumor-forming ability in athymic mice. Re-expression of the AR^{sr} 1/2/3/2b and AR^{sr} 1/2/3/CE3 isoforms, which have strong androgen-independent transcriptional activity (FIGS. 4 and 5), may promote high rates of cell proliferation and large tumors in castrated athymic mice. AR^{sr} 1/2/3/CE1 and AR^{sr} 1/2/3/CE2 also may promote proliferation in vitro and in vivo, but to a lesser extent. Re-expression of wild-type AR^{sr} may not have an effect on ADI growth in vitro or in vivo.

In addition, one may test transcriptional activities of individual and paired truncated androgen receptor isoforms to identify the molecular basis for differential transcriptional activity. Androgen receptor proteins dimerize at AREs in promoter and enhancer regions of target genes. The discovery of truncated androgen receptor isoforms containing both the DNA recognition helix and dimerization interface of the DBD suggests many possible combinations of homo- and heterodimers in ADI prostate cancer cells. Thus, one can test the transcriptional outcomes of pair-wise androgen receptor isoform expression.

Each of the five androgen receptor expression vectors used in FIG. 4 can be readily modified to contain either an in-frame N-terminal FLAG tag, or an in-frame N-terminal Myc tag. One can use these expression vectors in transient transfection experiments with the AR-null DU145 cell line, as well as the androgen-dependent LNCaP cell line, which does not express truncated androgen receptor proteins (Guo et al., 2009 Cancer Res 69:2305-13; Hu et al., 2009 Cancer Res 69:16-22). One can assess transcriptional activity through the use of luciferase reporters regulated by AR-responsive MMTV, 4×ARE-E4, or −5746-PSA promoters (FIG. 4). In DU145 transfections, one can assess all possible FLAG/Myc tagged androgen receptor homo- and heterodimer combinations (15 total). In LNCaP cells, one can co-express the four individual truncated androgen receptor isoforms alongside endogenous full-length AR, and also knock-down full-length androgen receptor with Exon 7-directed siRNA (FIG. 3B) and express pairs of FLAG/Myc tagged truncated androgen receptor isoforms (10 total). Cells may be cultured under androgen-free conditions for 48 hour post-transfection, and luciferase activity can be determined. For co-transfections involving full-length AR, luciferase activity can also be determined following 24 hours of treatment with 1 nM DHT. Transfections can be performed at least three times, in duplicate. Androgen receptor isoform expression can be monitored by Western blot using antibodies specific for, for example, the androgen receptor NTD, androgen receptor CTD, FLAG epitope, and/or Myc epitope. To ensure that the androgen receptor isoforms are dimerizing as expected in these experiments, one can perform chromatin immunoprecipitation (ChIP) and re-ChIP with FLAG- and Myc-specific antibodies in conjunction with commercially available PCR primer pairs that are specific for promoter reporter constructs. The protocol for interrogating transcription complexes on transiently transfected promoter-reporters using ChIP has been previously described [60]. One can also perform subcellular localization experiments by fractionating the nuclear and cytoplasmic compartments and assessing the localization of androgen receptor isoforms by Western blot with antibodies specific for, for example, the androgen receptor NTD, androgen receptor CTD, FLAG epitope, or Myc epitope.

In some cases, FLAG/Myc tagged homodimers exhibit transcriptional activities similar to those observed in FIG. 4. The AR 1/2/3/CE1 isoform may have compromised ligand-independent transcriptional activity. Thus, heterodimers involving this isoform may have reduced transcriptional activity.

AR Intragenic Rearrangement and Aberrant AR mRNA Splicing

Alternatively spliced, truncated AR isoforms support constitutive AR-mediated transcription and androgen-independent proliferation of 22Rv1 cells (Dehm et al., 2008 Cancer Res 68:5469-77; Guo et al., 2009 Cancer Res 69:2305-13; Hu et al., 2009 Cancer Res 69:16-22). We therefore examined whether these isoforms were also synthesized in CWR22Pc cells, a human prostate cancer xenograft cell line that is androgen-dependent for growth, which is in contrast to the CWR22-derived CRPCa 22Rv1 cell line.

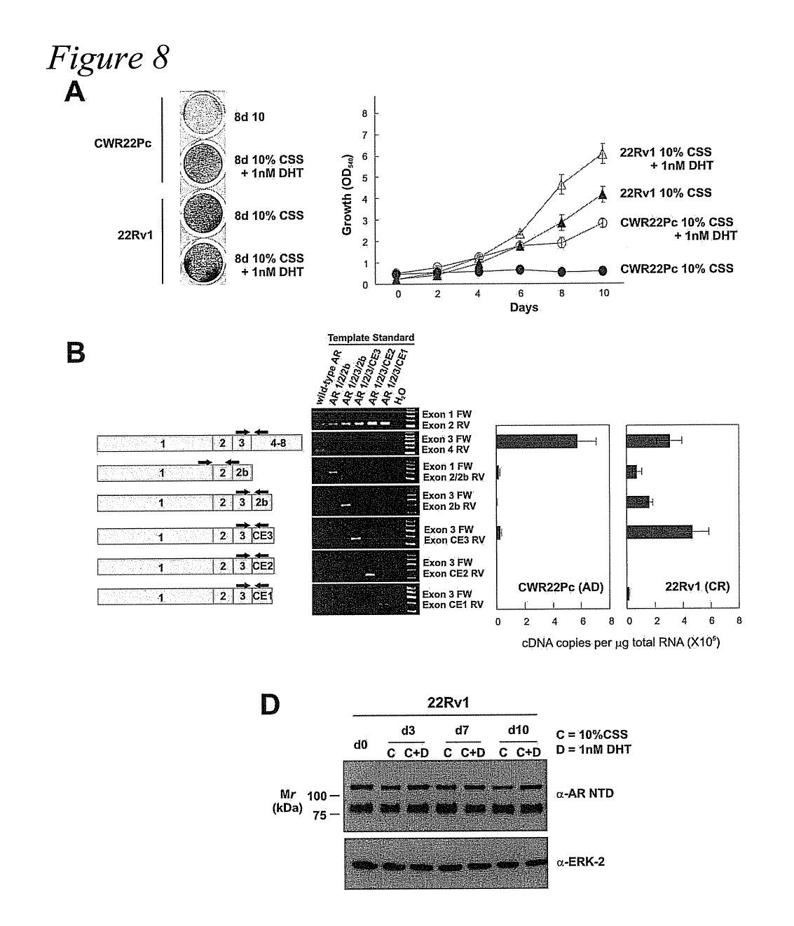

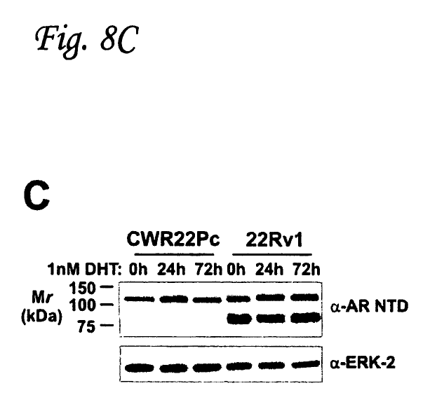

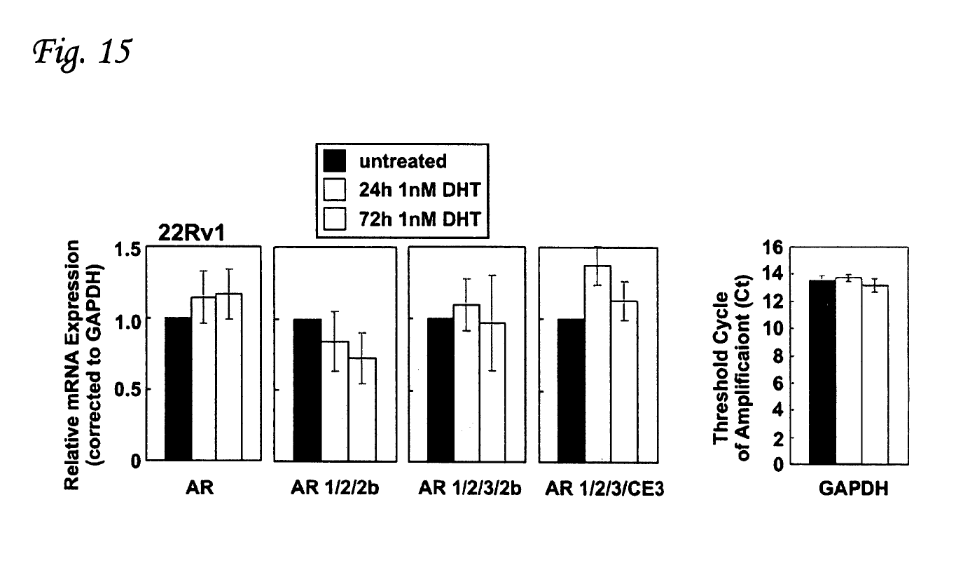

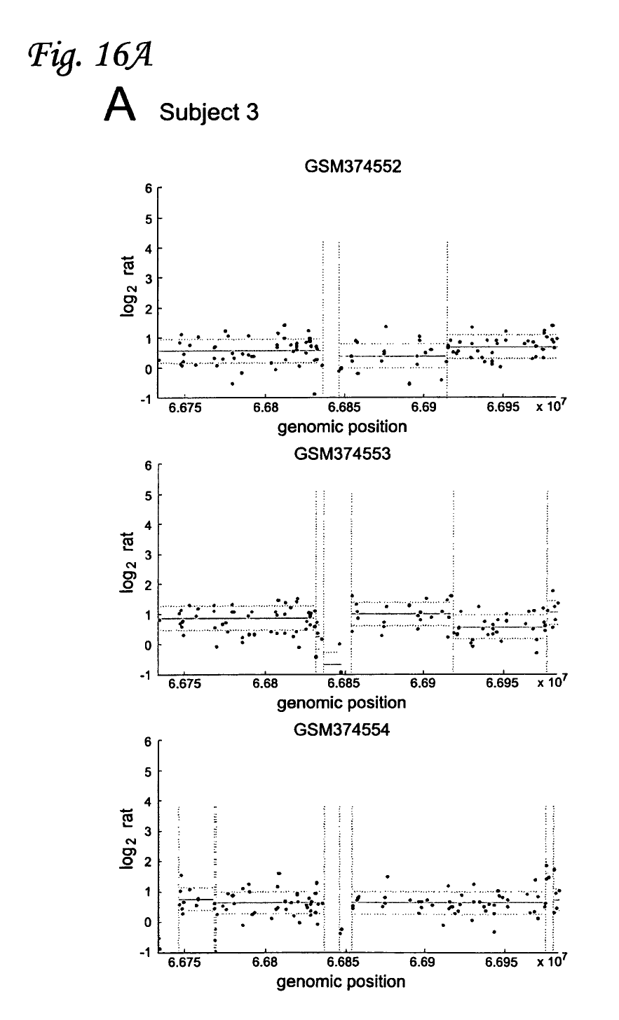

Using different PCR primer sets with different amplification efficiencies to identify the various AR mRNA isoforms precludes the use of the differential threshold cycle (Ct) of amplification (2^{-ΔΔCt}) method for determining relative expression by real-time PCR. We therefore pursued RT-PCR-based absolute quantification (FIG. 8B). Full-length AR expression as well as high-level expression of the AR 1/2/2b, AR 1/2/3/2b, and AR 1/2/3/CE3 isoforms was observed at the mRNA and protein level in 22Rv1 cells (FIG. 8B and FIG. 14). In androgen-dependent CWR22Pc cells, full-length AR expression was predominant, but expression of AR 1/2/3/CE3 mRNA and protein was also detectable (FIG. 8B and FIG. 15). No substantial change in these AR expression patterns was observed following 24 or 72 hours of treatment with androgens (FIG. 8C and FIG. 15). Similarly, AR isoform expression was stable during 10 days of 22Rv1 cell culture in the presence or absence of androgens (FIG. 8D). Together, these data demonstrate that both androgen-dependent CWR22Pc and CRPCa 22Rv1 cells can synthesize truncated AR isoforms, but 22Rv1 cells are able to sustain stable, high-level expression.

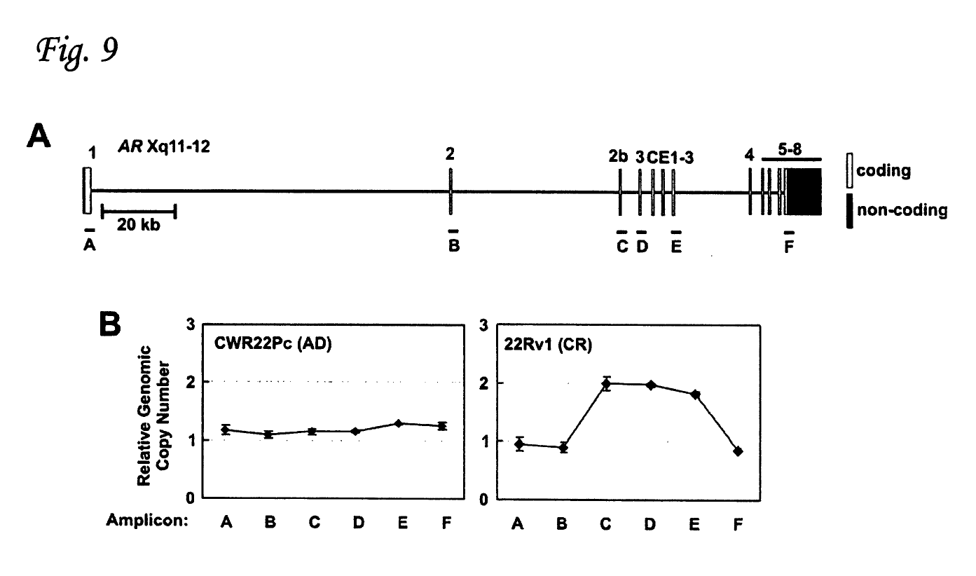

We next interrogated copy number at distributed loci along the length of the AR gene. Strikingly, in castration-resistant 22Rv1 cells, we observed increased copy number of AR Exons 2b, 3, and CE3, suggesting a rearrangement involving this genomic segment (FIG. 9). This aberration was not observed in the androgen-dependent CWR22Pc cell line (FIG. 9). These data therefore suggest that alternative AR isoforms, which support the castration-resistant phenotype of 22Rv1 cells, may arise via enhanced splicing of alternative exons harbored on a rearranged genomic segment in the AR locus.

### AR Intragenic Copy Number Alterations in Metastatic CRPCa Tissues

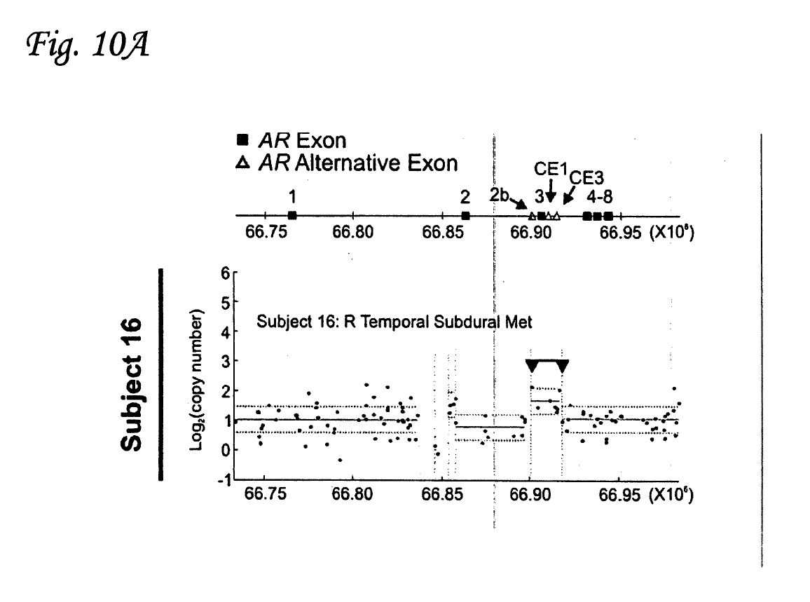

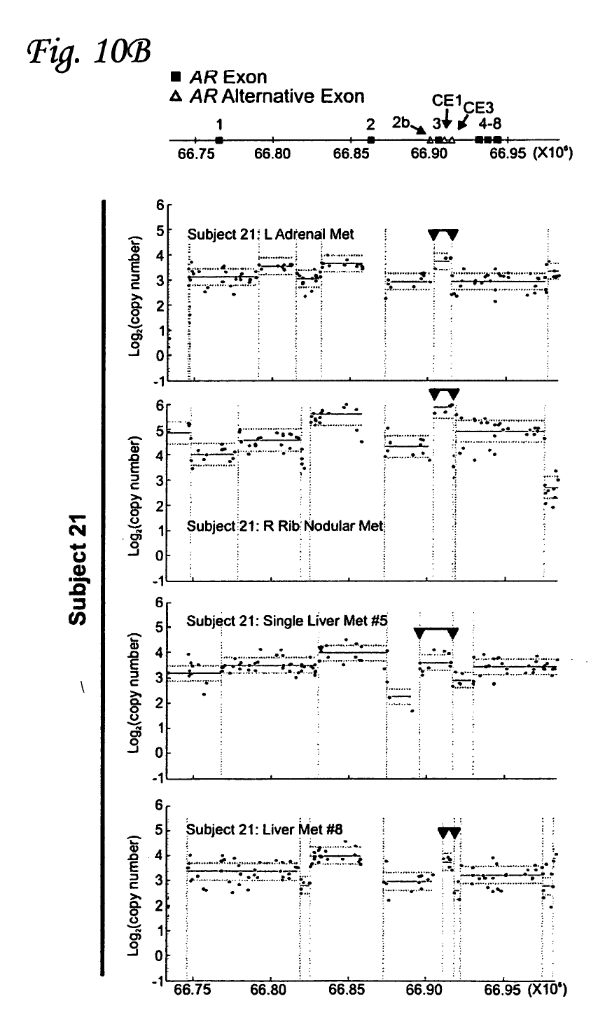

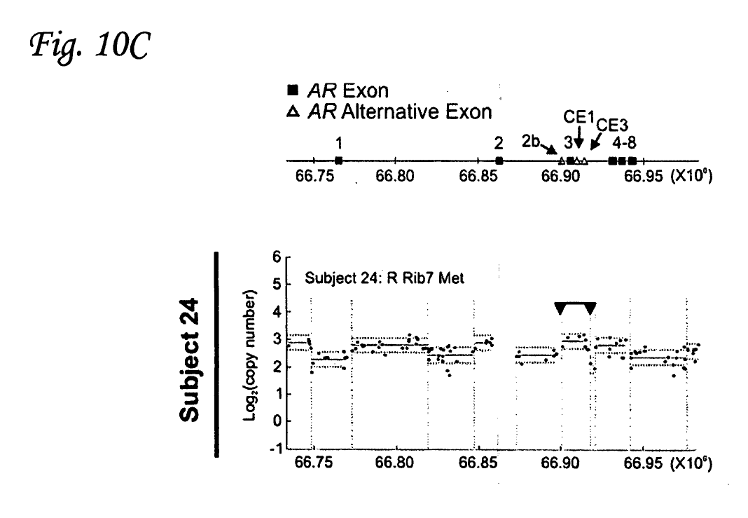

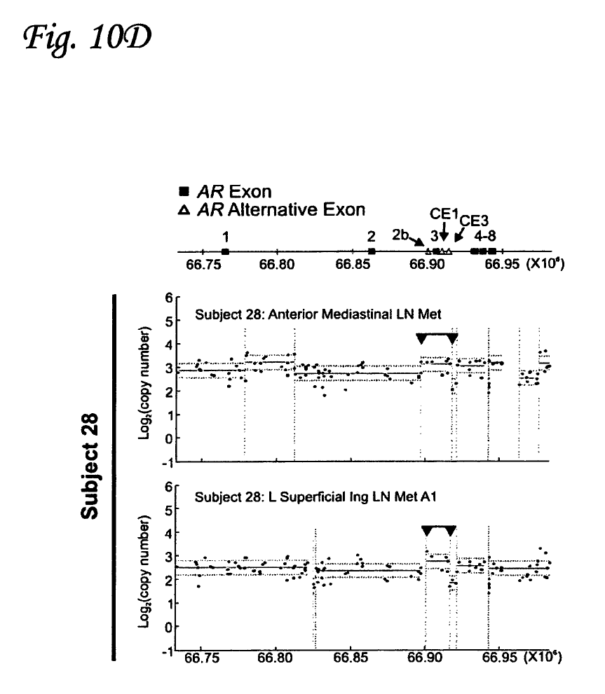

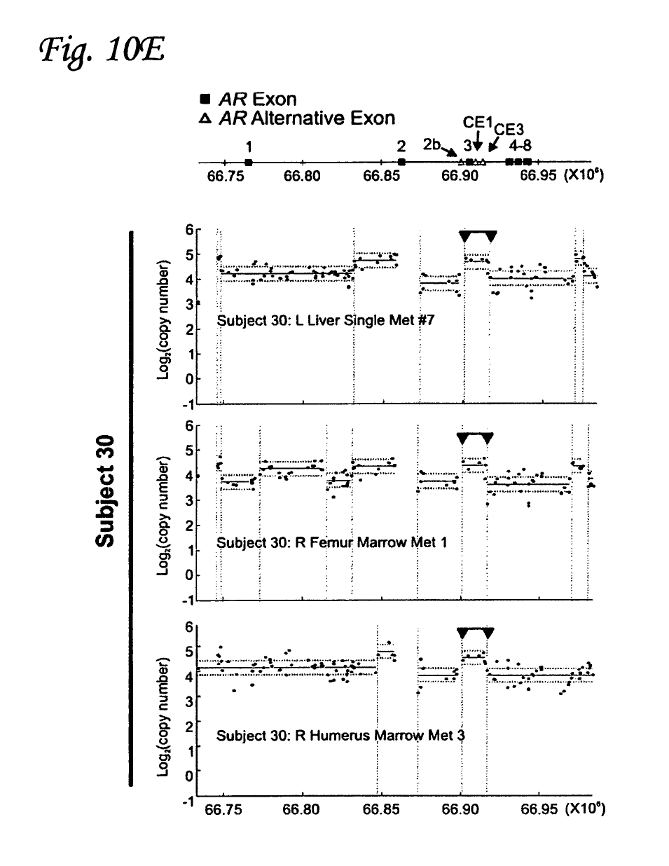

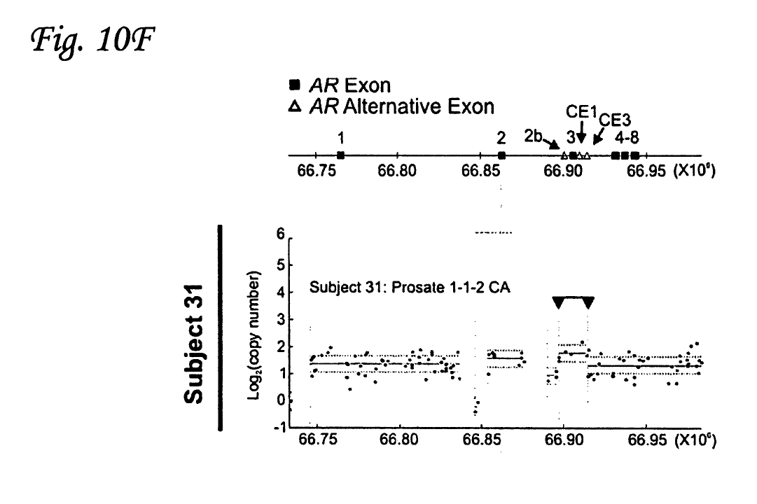

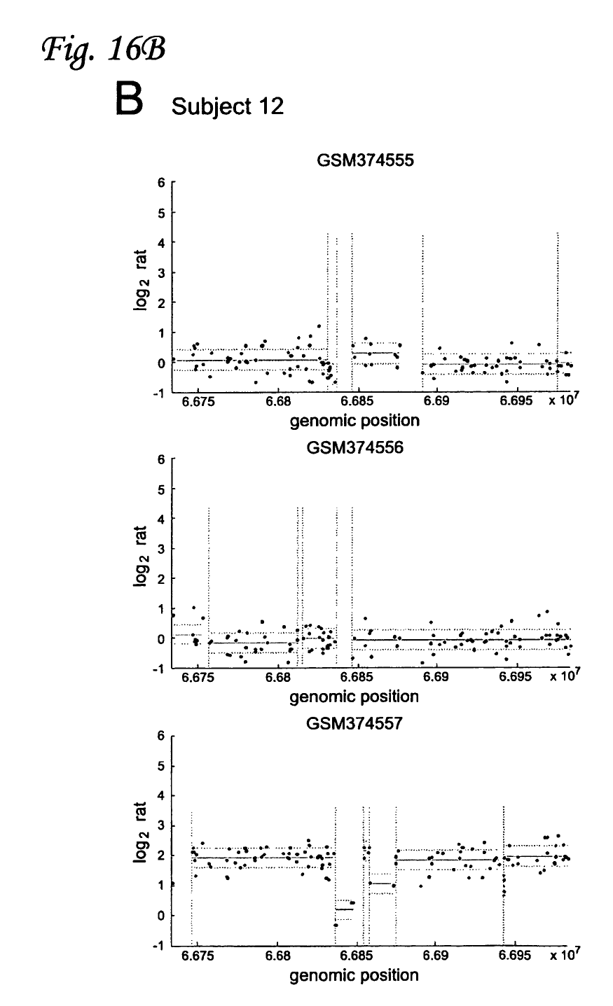

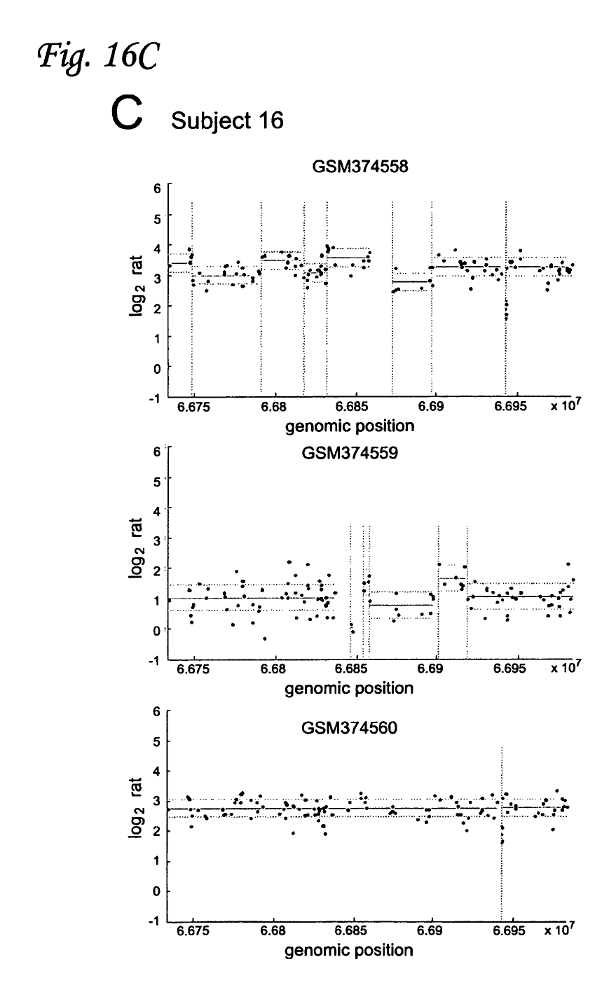

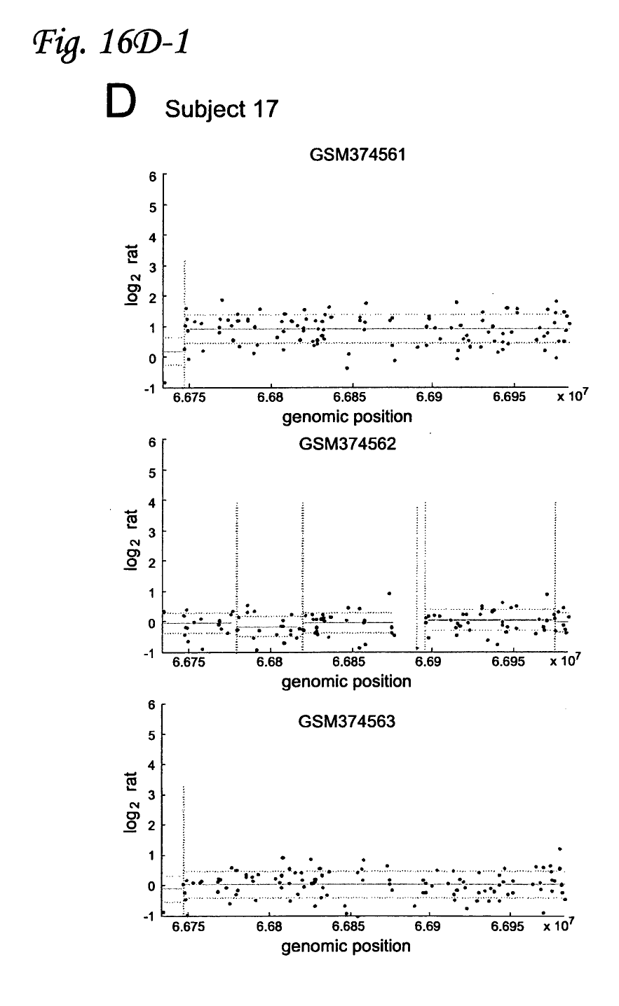

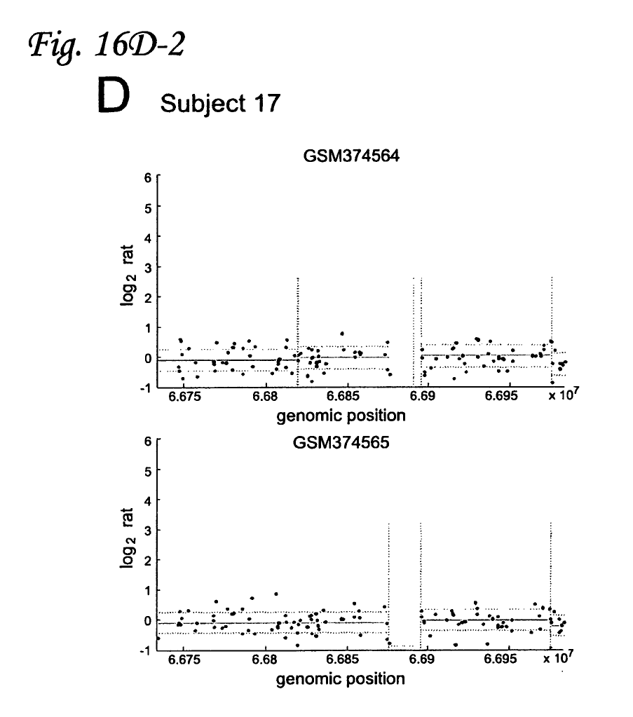

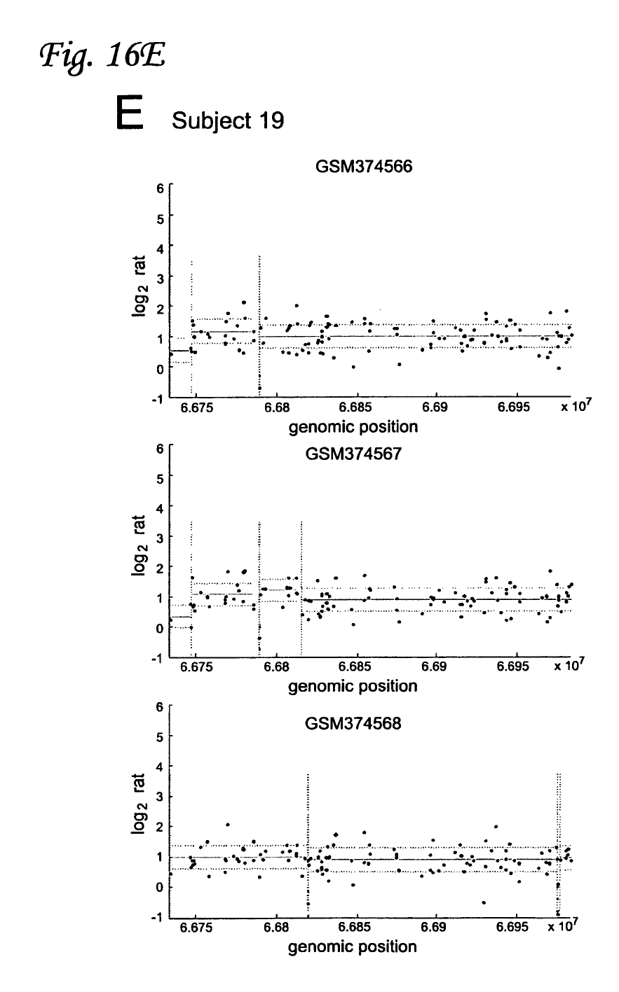

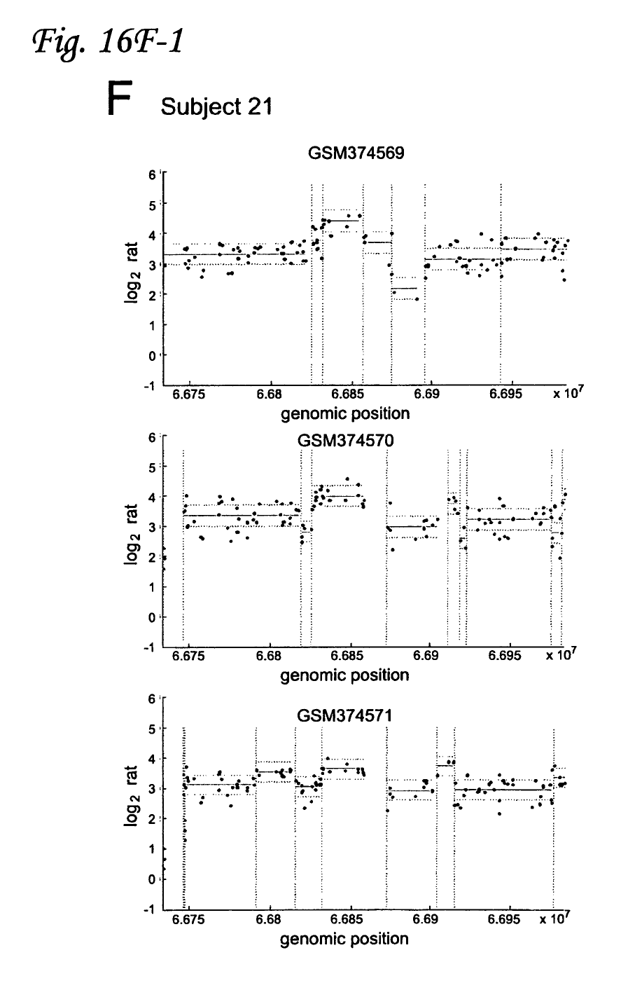

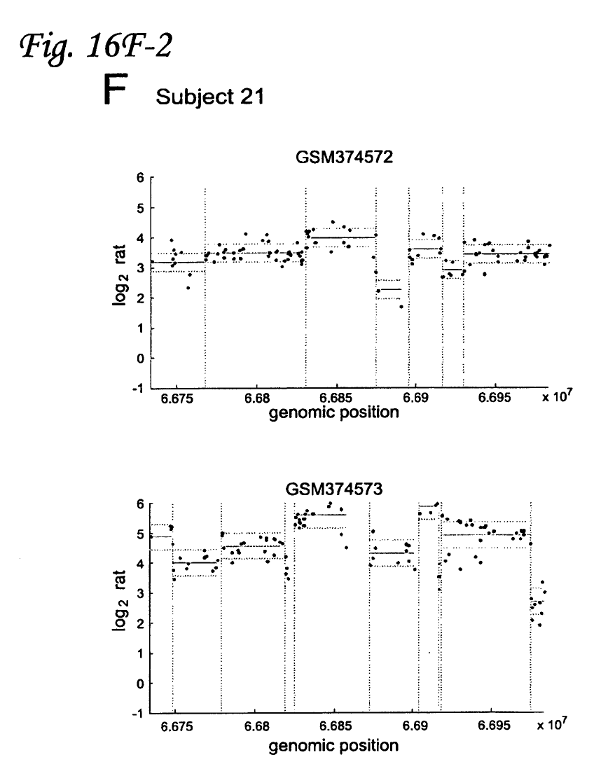

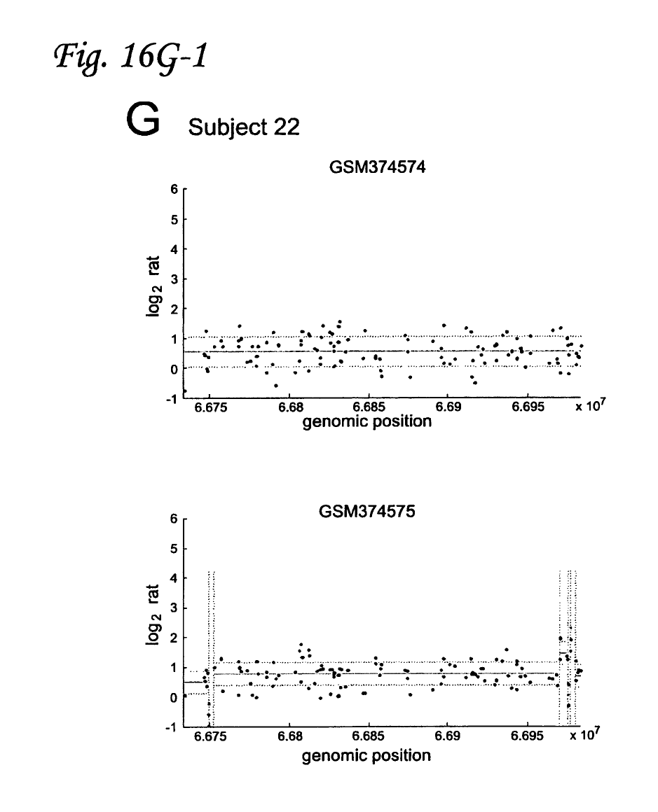

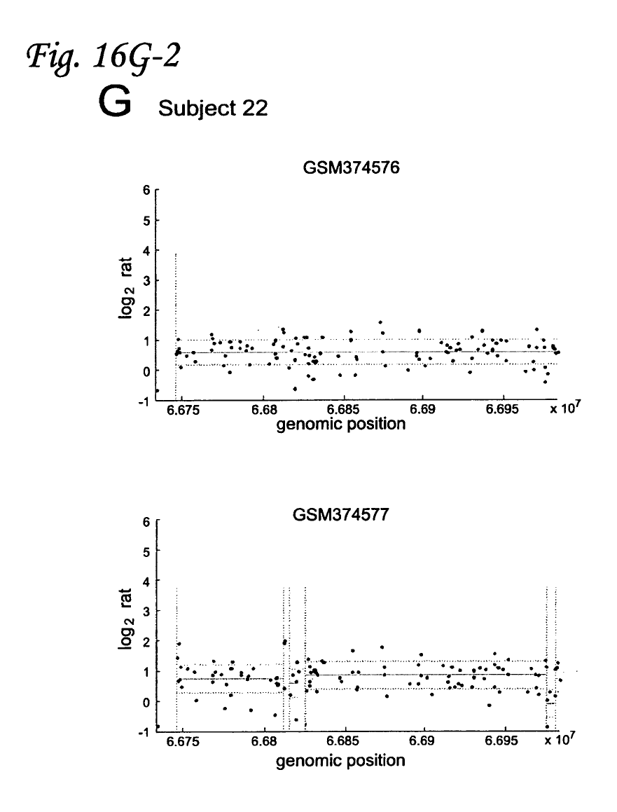

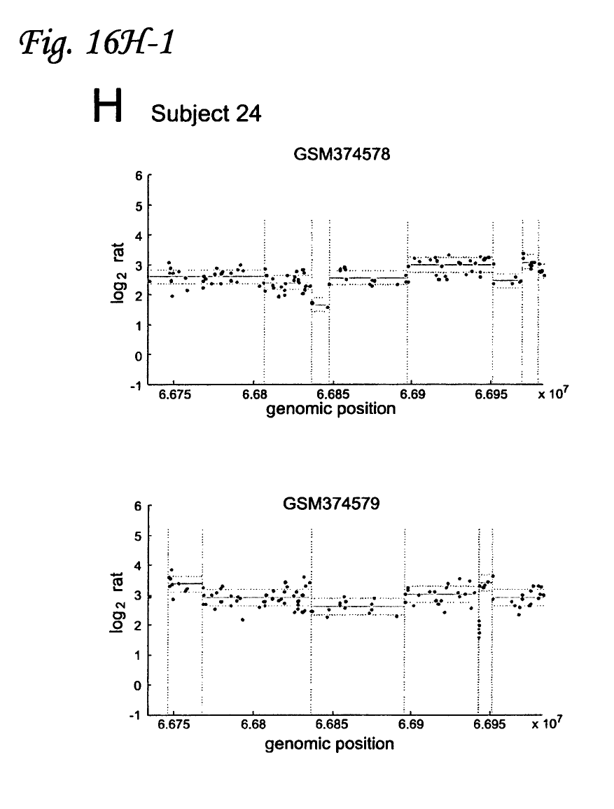

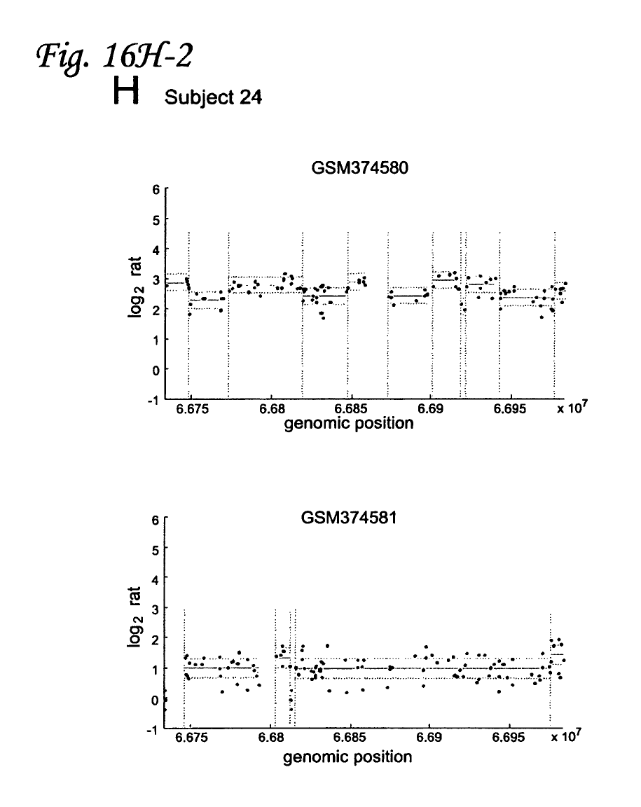

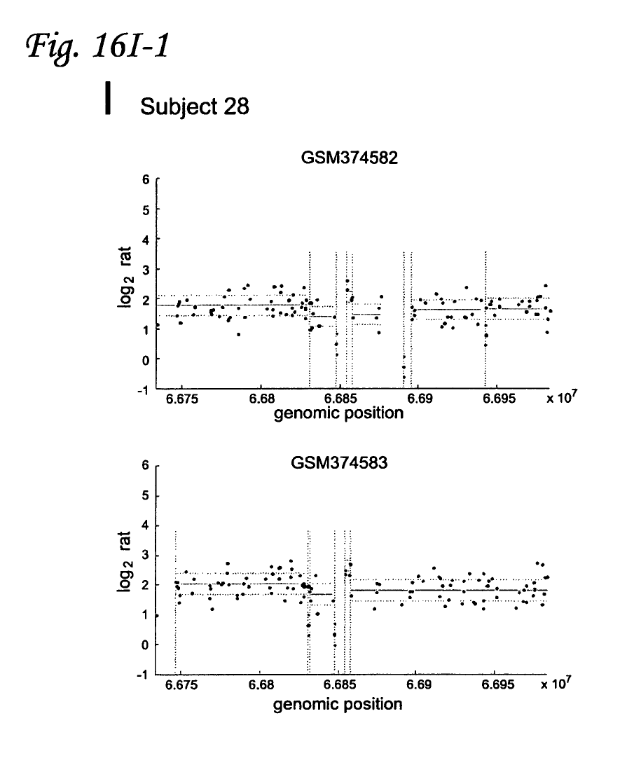

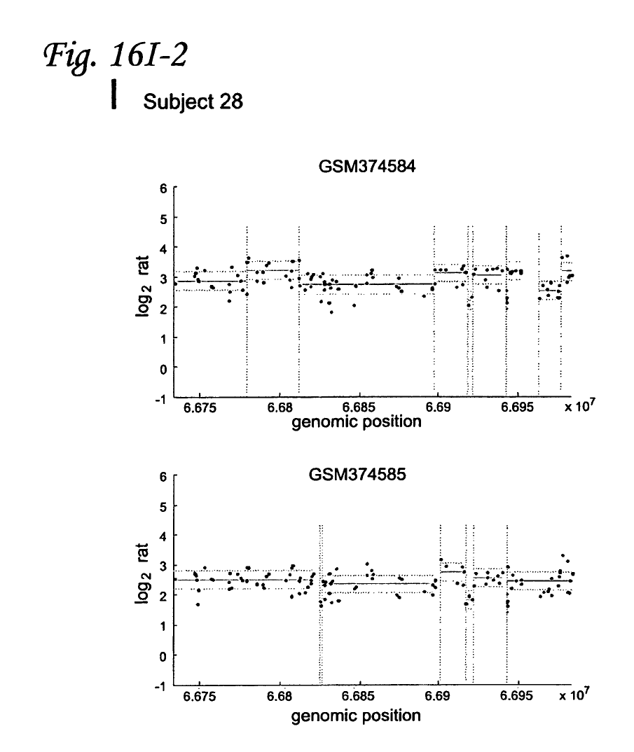

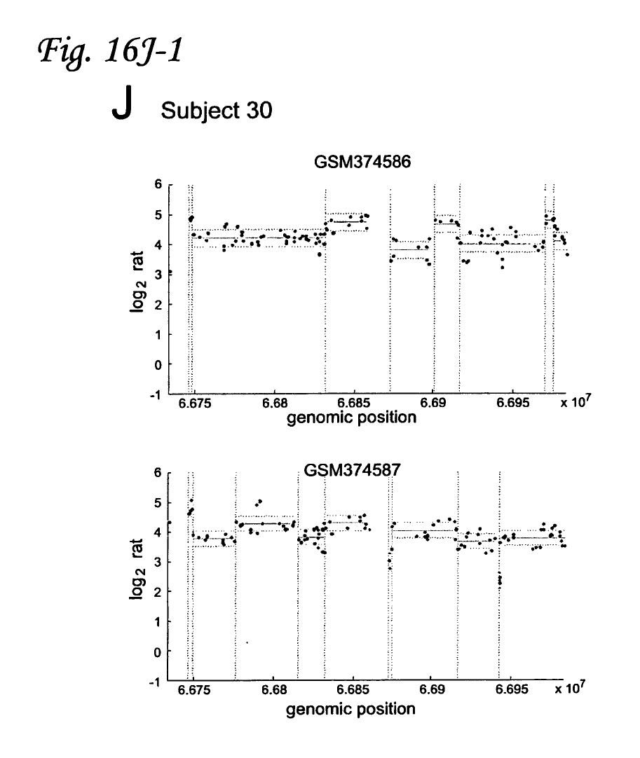

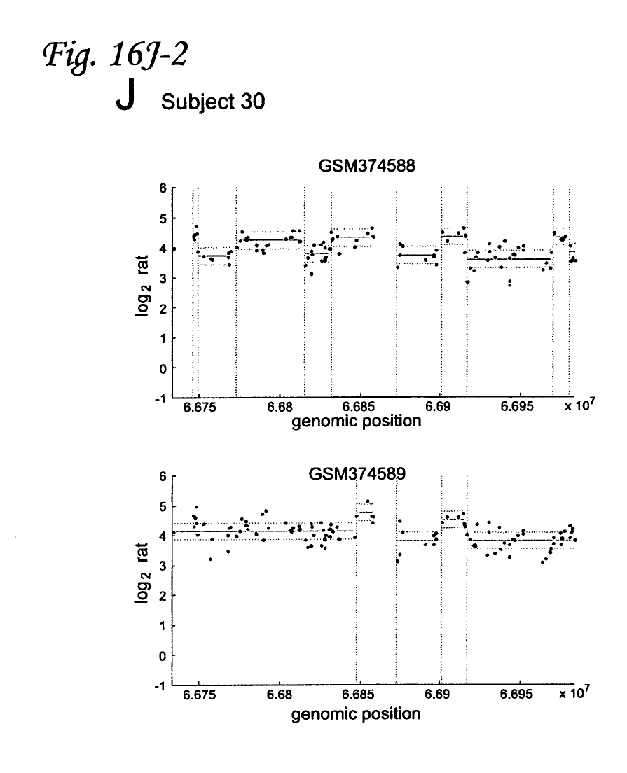

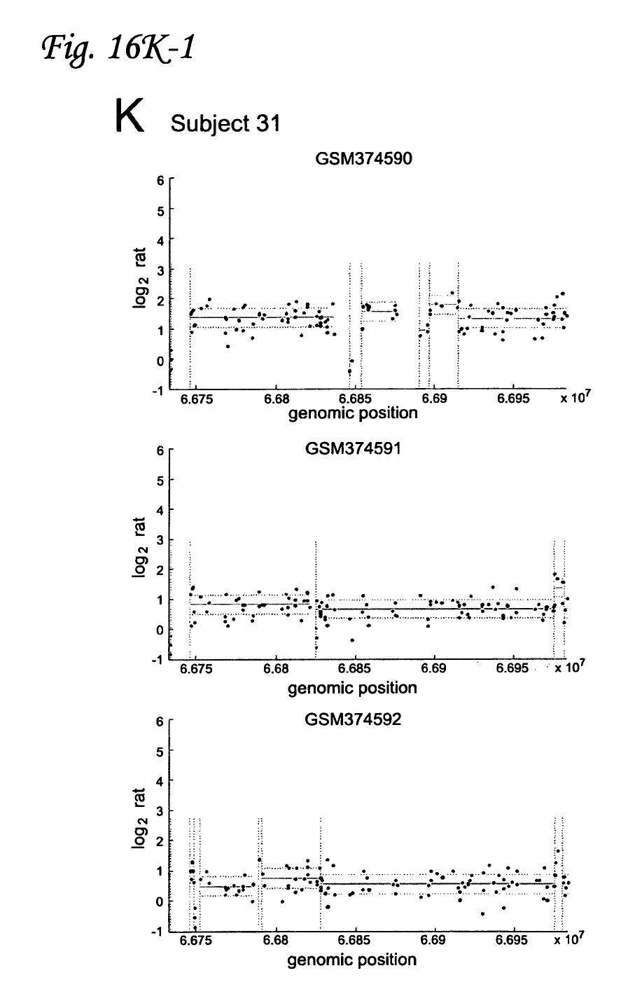

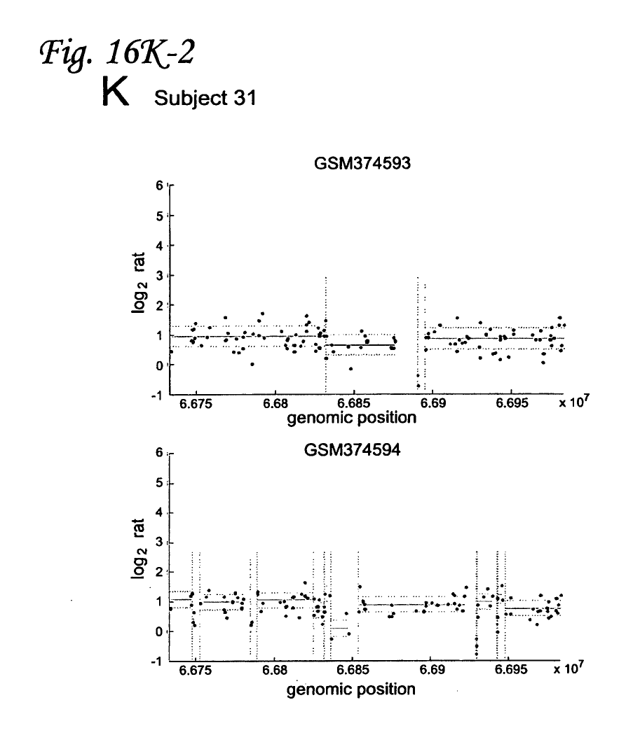

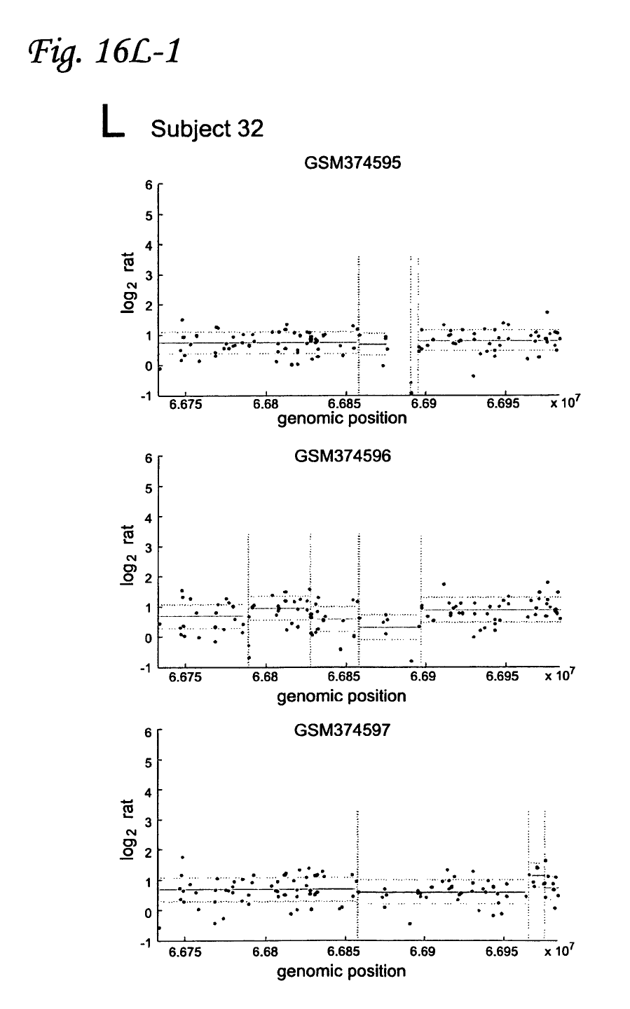

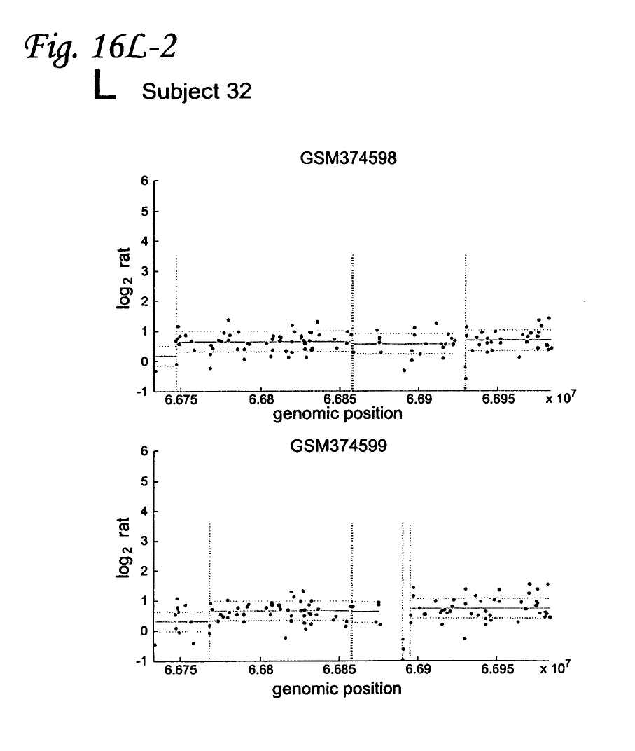

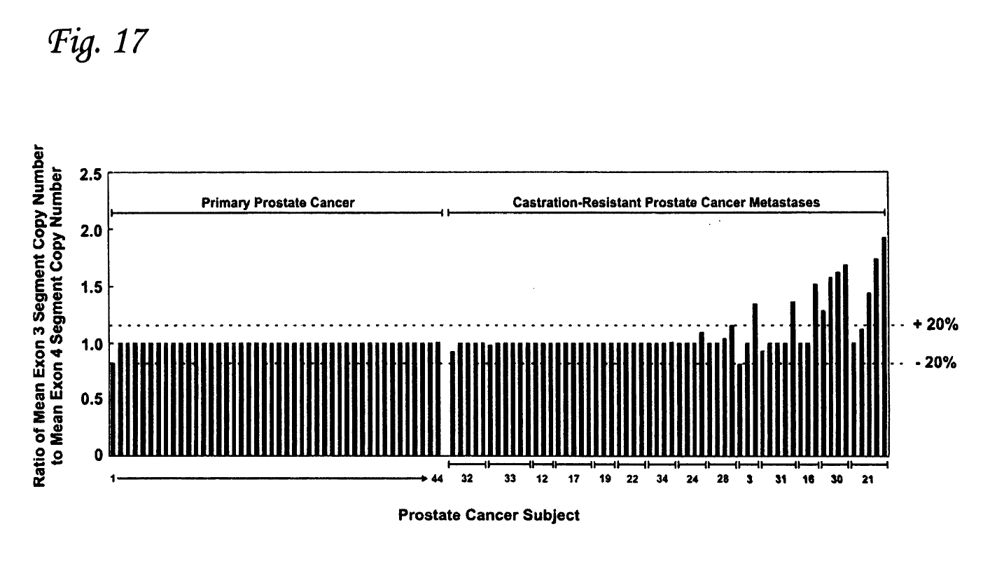

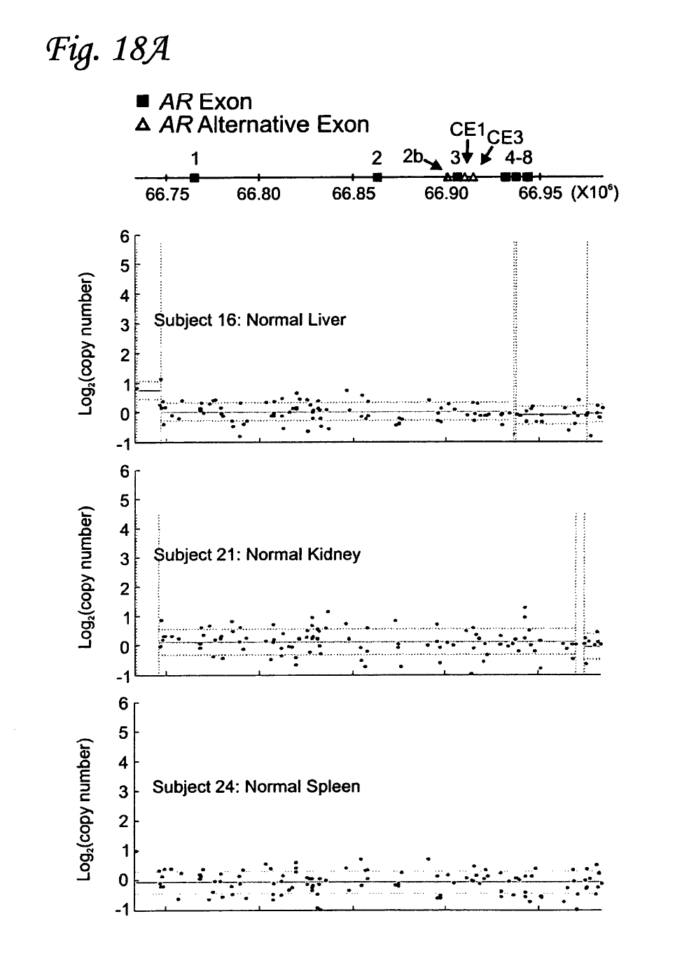

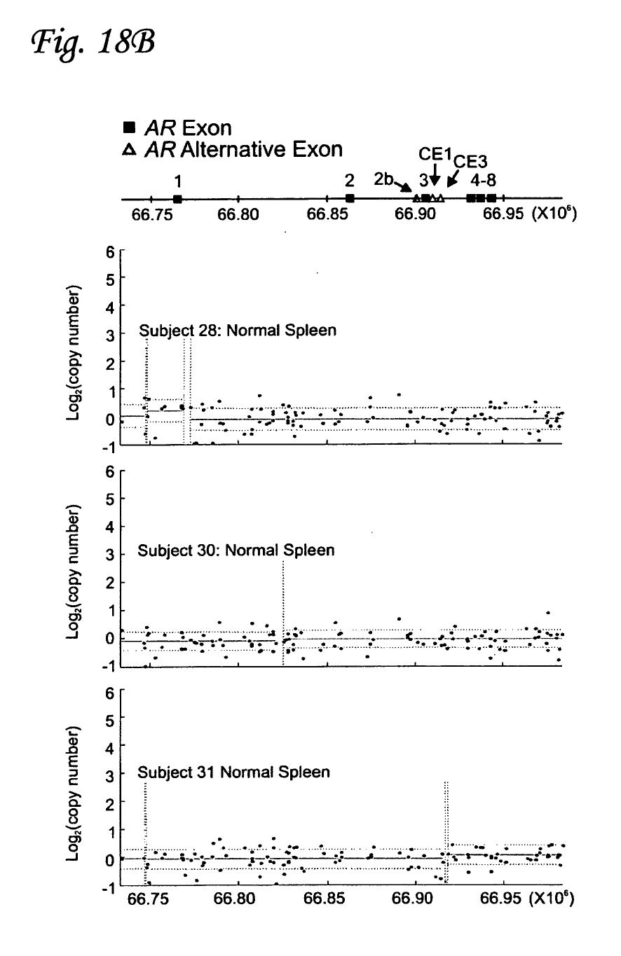

To determine whether AR intragenic copy number alterations occurred in human CRPCa, we analyzed high-resolution Affymetrix Genome-Wide Human SNP Array 6.0 (SNP6.0) data derived from clinical primary PCa (n=44 tissues from 44 patients) and metastatic CRPCa (n=58 tissues from 14 patients) (Liu et al., 2009 Nat Med 15:559-65; Mao et al., 2010 Cancer Res, 70:5207-12). To localize the boundaries of putative breakpoints, we used a dynamic program that estimates the number and locations of segments adaptively based on probe-level data. This analysis revealed a high incidence of rearrangement in conjunction with AR amplification, only in CRPCa, which to our knowledge is a novel phenomenon that has not been described (FIG. 16). Because the outcome of the 22Rv1 AR rearrangement appeared to be a focal copy number increase of a segment between AR Exons 2 and 4, resulting in higher dosage of Exon 3 and alternative exons relative to AR Exon 4 (FIG. 9), we asked whether these phenomena occurred in clinical PCa. Indeed, focal copy number increases were observed between AR Exons 2/3 and 3/4 in 12/58 (20.7%) metastases from 6/14 (42.9%) subjects, which presented as rearrangement of a segment harboring some or all alternative AR Exons 2b or CE1-3 (FIG. 10). For most of these CRPCa samples, the outcome was higher gene content of a segment containing AR Exon 3 and alternative exons compared with AR Exon 4 (FIG. 17). These alterations were not observed in genomic DNA samples from these subjects' normal tissue (FIG. 18). Focal copy number increase of this segment in one CRPCa subject was confirmed using targeted quantitative PCR (FIG. 19). SNP6.0 analysis revealed no changes in overall AR copy number or focal alterations in this region in 44 primary PCa samples (FIG. 17), indicating that CRPCa patients are more likely to harbor this rearrangement in at least one of their tumors than patients with localized, androgen dependent PCa (6/14 vs. 0/44, P=0.000074, Fisher's exact test). Overall, these data suggest that the region encompassing AR Exon 3 may represent a “hotspot” for intragenic rearrangement in CRPCa.

Comparative DNA copy number observations can be modeled as a constant function with transitions whose locations and amplitudes are unknown. The amplitude of the constant function is 1 whereas the transitions' amplitudes can be any positive number greater or less than one. Values greater than 1 indicate duplication and values less than 1 indicate deletions. Taking the log 2 ratio of the observations centers the normal observations on zero and makes the values of duplications and deletions greater or less than zero, respectively.

The cDNA observations are usually corrupted with non-parametric signal-dependent noise which requires the data to be normalized before being analyzed. In this study, the samples were normalized by centering the samples' geometric mean intensity to one (0 in log space). This modifies the observations to be consisting of a constant function with transitions corrupted by white Gaussian noise.

Formally, N observations can be considered as:

Y[n]=F[n]+W[n], n=0, 1, 2, . . . , N−1 (1)

Y[n] is the observed noisy signal, and W[n] is the additive white noise. The challenge is to extract the noise-free signal F[n] from the observation Y[n]. F[n] consists of M successive segments, each segments has unknown start, end, and mean value.

$$F [ n ] = \{ \begin{matrix}{A}_{1} & n = {n}_{0} , 1 , … , {n}_{1} - 1 , \\ {A}_{2} & n = {n}_{1} , {n}_{1} + 1 , … , {n}_{2} - 1 , \\ ⋮ & \\ {A}_{M} & n = {n}_{M - 1} , {n}_{M - 1} + 1 , … , N - 1.\end{matrix}$$

The abnormality of a segment n is proportional to the deviation between its mean (Aₙ) and the value zeros. It is of note that n₁<n₂< . . . <n_{M}.

CGH segmentation (Picard et al., 2005 BMC Bioinformatics 6:27) exhibits the principle of maximum likelihood estimator to estimate F[n] by breaking the observations Y[n] into constant segments with different mean values. The likelihood between Y[n] and F[n] is measured as P(Y[n]/F[n]), and this measurement can be expressed, considering the white Gaussian noise, as the following:

$$L ( n ) = {\prod }_{i = 1}^{M} {\prod }_{j = {n}_{i - 1}}^{{n}_{i} - 1} \frac{1}{\sqrt{2 {πσ}_{i}^{2}}} {e}^{\frac{- {( Y [ j ] - {A}_{i} )}^{2}}{2 {σ}^{2}}} \qquad ( 2 )$$

Assuming the noise variance to be uniform across the observation reduces eq(2) to:

$${L}_{1} ( n ) = {\prod }_{i = 1}^{M} {\prod }_{j = {n}_{i - 1}}^{{n}_{i} - 1} {e}^{\frac{- {( Y [ j ] - {A}_{i} )}^{2}}{2 {σ}^{2}}} \qquad ( 3 )$$

Taking the log value of eq(3) yields:

$$log \{ {L}_{1} ( n ) \} = {\sum }_{i = 1}^{M} {\sum }_{j = {n}_{i - 1}}^{{n}_{i} - 1} \frac{- {( Y [ j ] - {A}_{i} )}^{2}}{2 {σ}^{2}} \qquad ( 4 )$$

This equation is equivalent to the equation of Mean Square Error between the observations Y[n] and the noise-free signal F[n]. It is one of the Gaussian noise characteristics where the maximum-likelihood and Minimum Mean Square Error (MMSE) estimators are equivalent.

Eq(4) is concave on the number of segment, M, which guarantees that the MMSE keeps decreasing until it reaches zeros when M=N. In this case, the number of segments is equal to the number of data points and each segment will consist of only one data point. This result is useless and it must be avoided by employing two constraints: a maximum number of segments suggested by the user, and a penalty function to avoid the unnecessary growth of the total number of segments. These constrains modifies eq(4) to be:

$$MSEE ( M ) = M log ( N ) + {\sum }_{i = 1}^{M} {\sum }_{j = {n}_{i - 1}}^{{n}_{i} - 1} {( Y [ j ] - {A}_{i} )}^{2} \qquad ( 5 )$$

Therefore, the algorithm breaks the observations into M segments testing all possible breakpoints and measuring the MSEE value using this collection of breakpoints. The required number of calculations is 2^{N} which grows very fast. It is impossible to test all these permutations, and therefore, CGH segmentation algorithm exhibits a dynamic program to reduce the amount of calculations to M·N which provides a significant reduction in the computational load.

The idea of the dynamic program is to provide one new breakpoint recursively. It tests all N observations and measures the total sum of square error (SE) of the observations from 1 to the tested point, and the (SE) of the rest of the observations. The point that provides the minimum sum of square error is chosen to be the new breakpoint and then the dynamic program starts the search for the next one. To rephrase that in equations, let

$${SE}_{i} ( a , b ) = {\sum }_{n = a}^{b} {( Y [ n ] - {F}_{i} [ n ] )}^{2} \qquad ( 6 )$$

When the dynamic program starts (no breakpoints are found yet), it assigns zeros to all F[n] values. After finding a new breakpoint, the values of F[n] will be revised. All F[n] values between every two successive breakpoints will be assigned the average value of the observations, Y[n], located at that interval.

In equation (6), the variable i varies from 1 to the maximum number of segments (provided by the user), and MSEE can be measured as the following:

$${MSEE}_{i} = {min}_{h} \{ {SE}_{i} ( 1 , h ) + {SE}_{i} ( h + 1 , N ) \} \qquad ( 7 )$$

Then, the penalty function {i log(N)} is added to each value of MSEEᵢ and the Minim Mean Square Error (which is equivalent to the Maximum Likelihood Estimator) will be:

$$MMSEE = {min}_{i} \{ {MSEE}_{i} + i log ( N ) \} \qquad ( 8 )$$

Eq(8) indicates that to obtain the MMSEE, it is not necessary to break the observations into M segments. This is obvious since the Bayesian Information Criterion (BIC) penalty function is linearly increasing while MSEEᵢ is downward concave.

In this study, the M value chosen for the X chromosome was 4800 segments. This was based on previous Partek Genomics Suite analysis of this SNP6.0 data set deriving 52,227 segments for the entire ˜3000 Mbp genome, for an average of ˜50 kb/segment (Liu et al., 2009 Nat Med 15:559-65). Because we were interested in identifying putative segments <50 kb, our rationale was that doubling the maximum number of possible genomic segments to 104,454 would make for an average of ˜25 kb/segment. On this basis, the maximum number of possible segments on the 155 Mbp X chromosome would be ˜5000.

### AR Breakpoint Junction Boundaries Lie Within LINE-1 Elements in 22Rv1 Cells

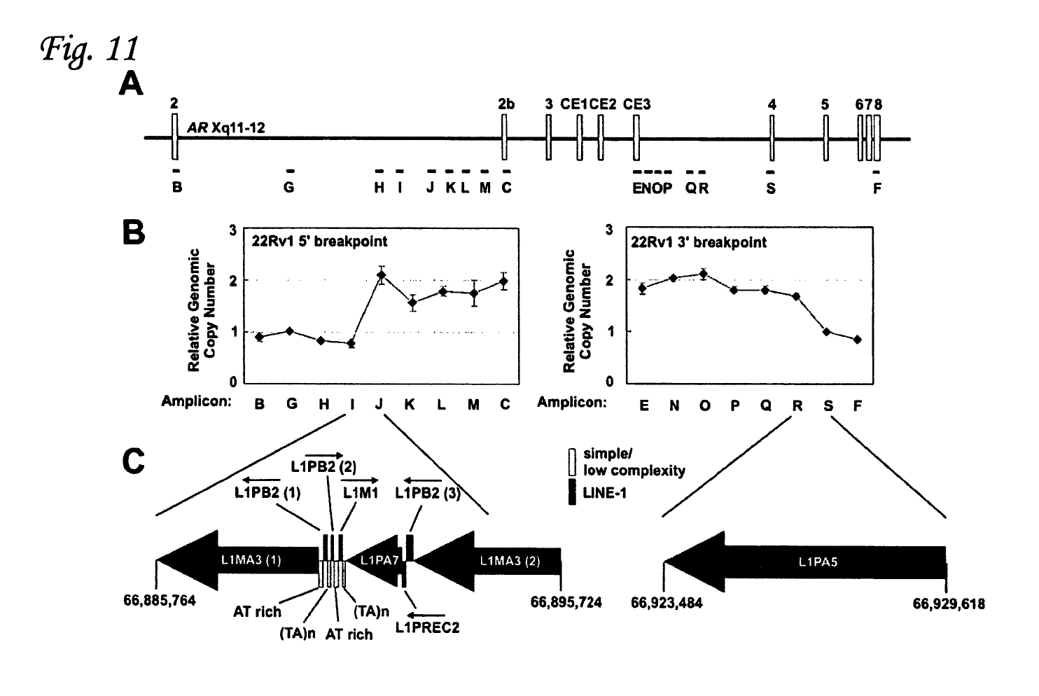

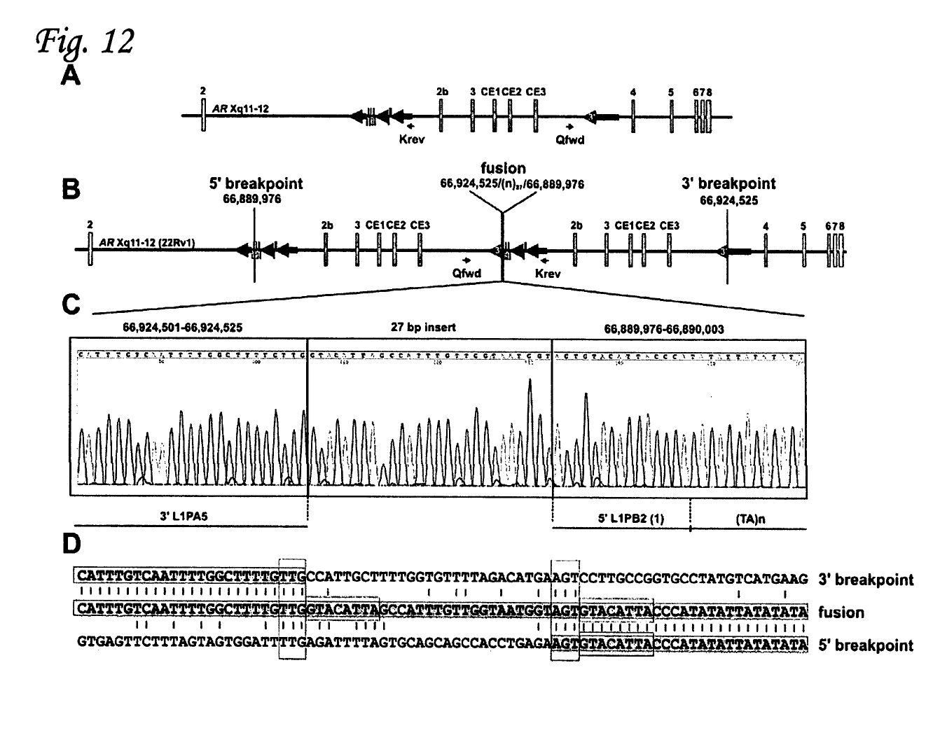

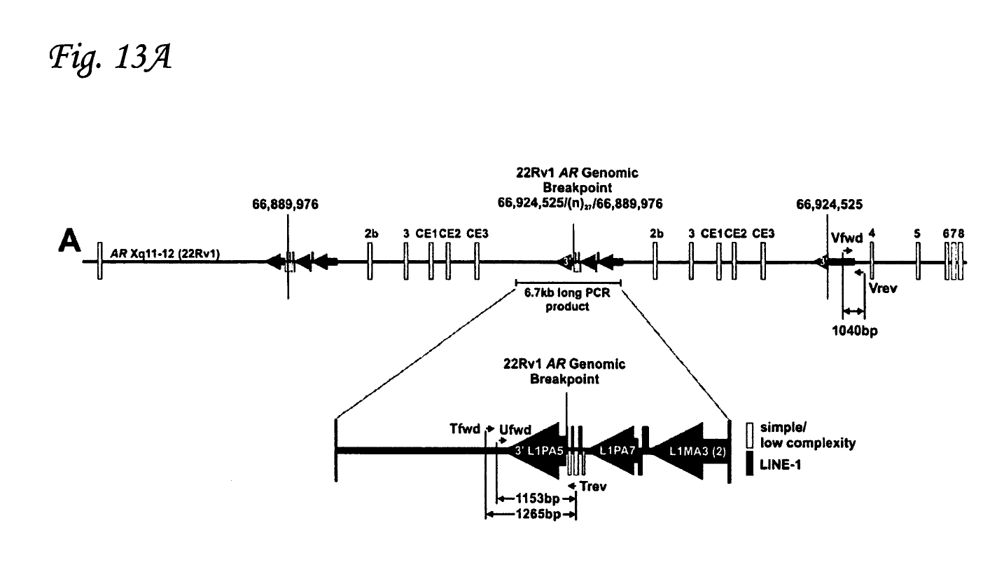

To establish with more precision the breakpoint junctions between AR Exons 2 and 2b as well as AR Exons CE3 and 4 in 22Rv1 cells, we carried out higher-resolution copy number interrogation (FIG. 11A). Using this approach, we mapped the 5′ breakpoint between AR Exons 2 and 2b to a resolution of 4 kb (FIG. 11B). Concurrently, we mapped the 3′ breakpoint between AR Exons CE3 and 4 to a resolution of 8 kb (FIG. 11B). Attempts to map the 5′ or 3′ breakpoints with higher resolution yielded real-time PCR products associated with very low Ct values in both reference and test DNA samples, indicating repetitive sequence. Indeed, analysis of public reference genome sequence revealed long interspersed nuclear elements (LINE-1) and low complexity (TA)n repeats and AT rich sequence in both of these regions (FIG. 11C).

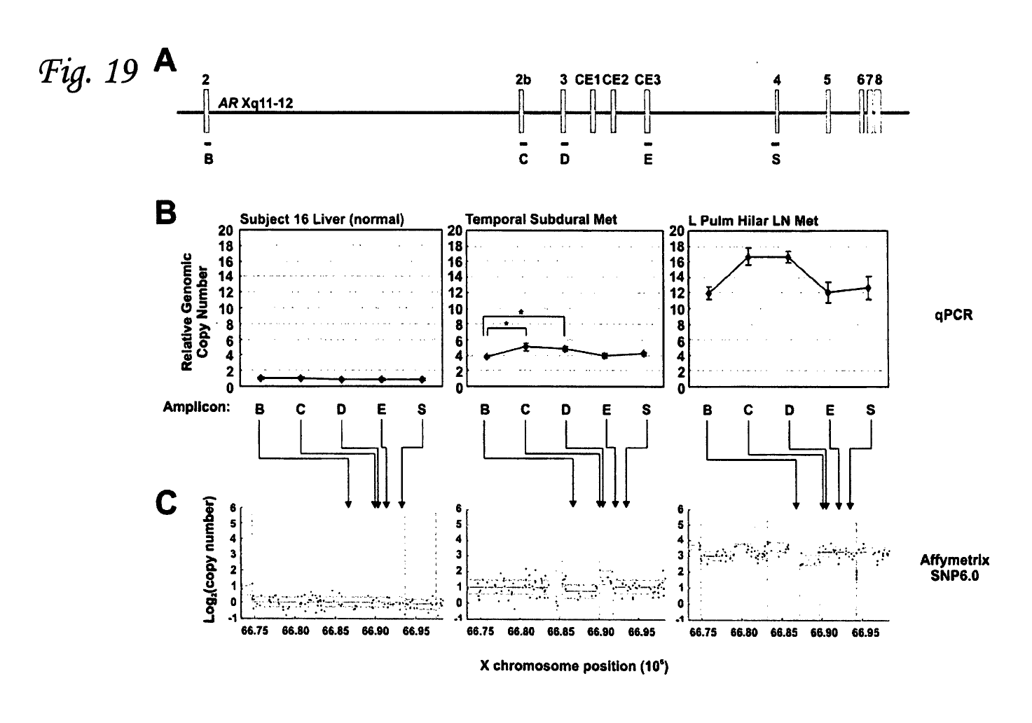

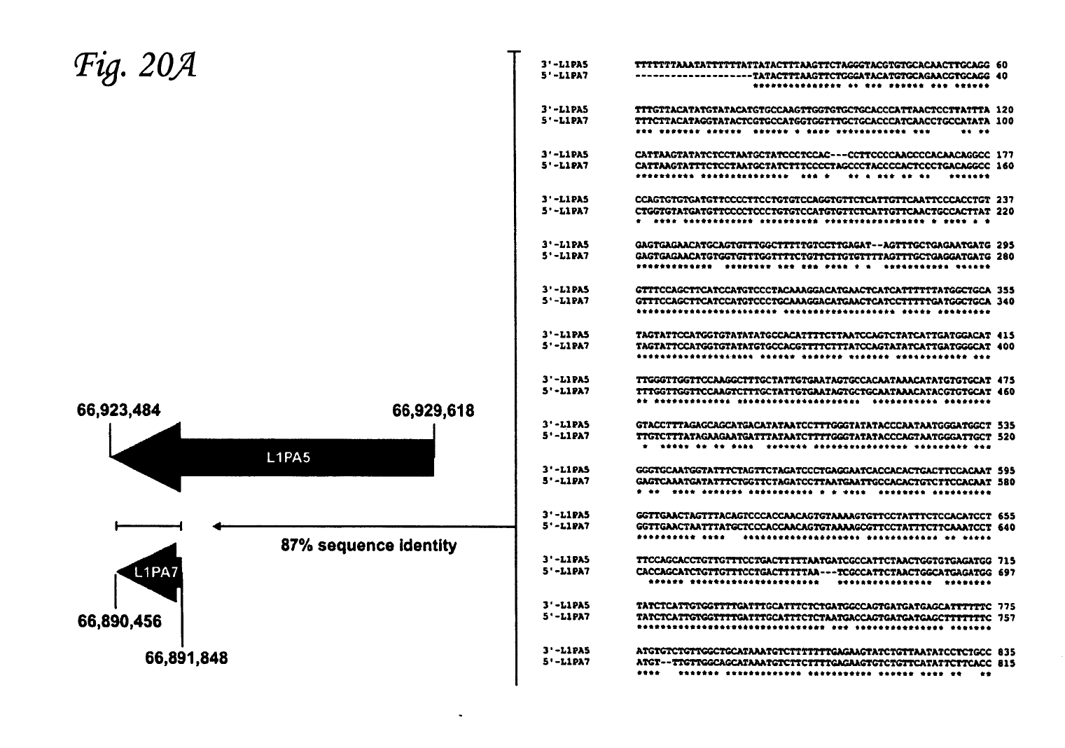

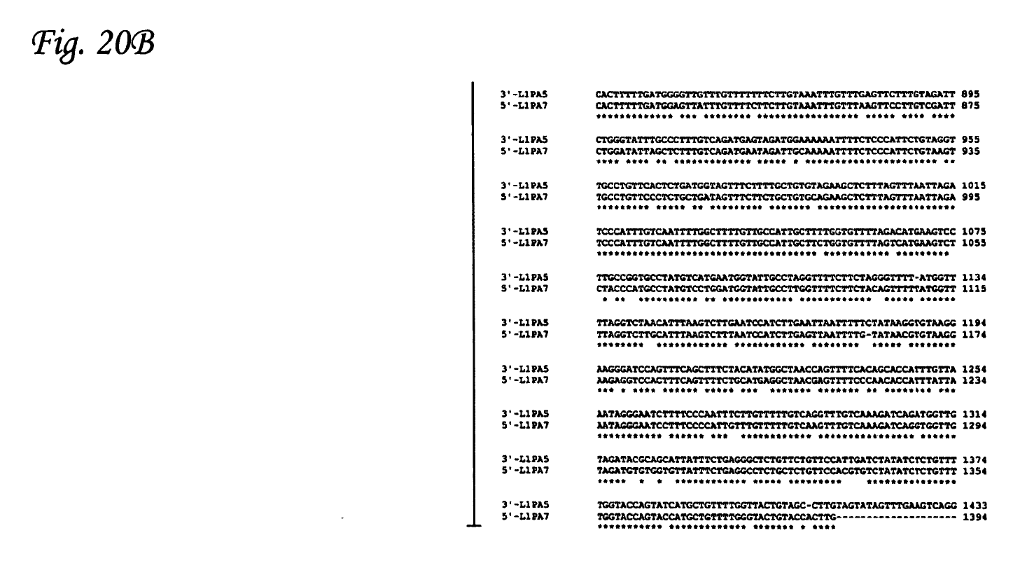

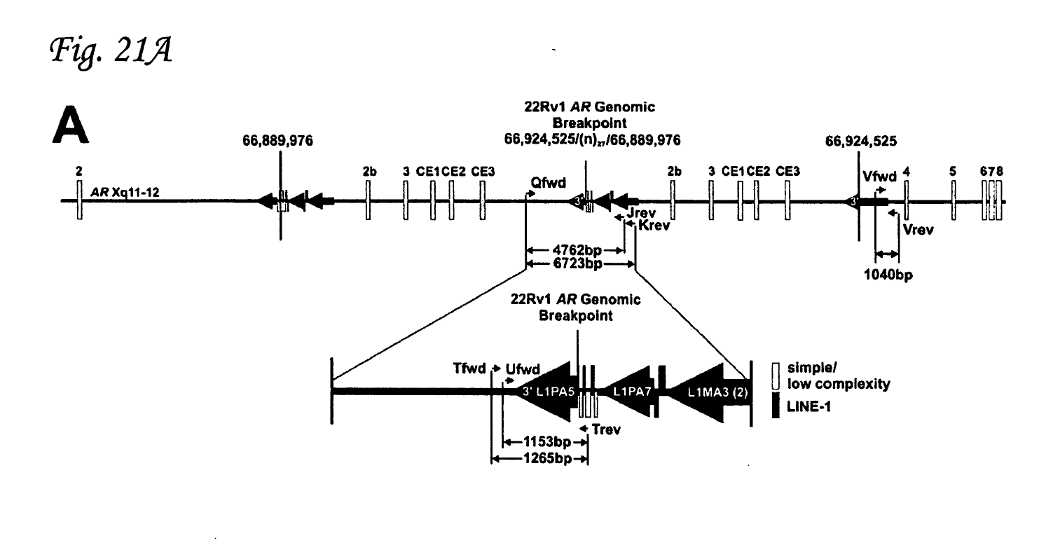

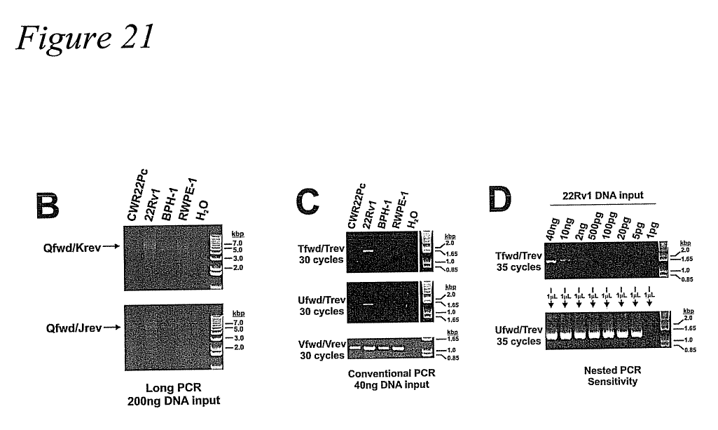

It is common for the endpoints of genomic deletions or insertions to map to repetitive elements such as LINE-1, although the underlying mechanisms are not fully-established (Belancio et al., 2008 Genome Res 18:343-58; Cordaux and Batzer, 2009 Nat Rev Genet 10:691-703). One possibility is that extensive homology between LINE-1 elements at breakpoint junctions could lead to deletion on one sister chromatid and duplication on the other via non-allelic homologous recombination (NAHR) (Hastings et al., 2009 Nat Rev Genet 10:551-64). Pairwise alignments between the 5′ LINE-1 fragments and the full-length 3′ LINE-1 element identified a >1 kb stretch of 87% sequence identity with one particular 5′ LINE-1 fragment, implicating NAHR as the basis for this rearrangement (FIG. 20). Therefore, we performed long-range PCR using two pairs of outward facing primers to isolate the breakpoint junction in 22Rv1 cells (FIG. 12A and FIG. 21). This resulted in long PCR products of 6723 and 4762 (FIG. 21). Sequencing of cloned PCR products revealed that they were identical over the common 4762 bp, and localized the 22Rv1-specific 5′ and 3′ breakpoints to genomic positions 66,889,976 and 66,924,525, respectively (FIGS. 12B and C). Analysis of the break fusion junction revealed 27 bp of inserted sequence (FIG. 12C). The origin of this sequence was not apparent by BLASTN and BLAT searches; however, the first 8 bp of this sequence perfectly matched an 8 bp motif at the 5′ breakpoint. Sequence alignments of the cloned break fusion junction and the 5′ and 3′ breakpoints demonstrated virtually no extended homology through this region (FIG. 12D). However, regions of 3 bp microhomology were found at the breakpoints (FIG. 12D). Microhomology at the breakpoints, as well as inserted sequence at the fusion site argues against NAHR and supports a microhomology-mediated break-induced replication (MIMBIR) (Hastings et al., 2009 PLoS Genet 5:e1000327) mechanism of segmental duplication in 22Rv1 cells.

### Emergence of CRPCa Cells During Long-Term CWR22Pc Castration

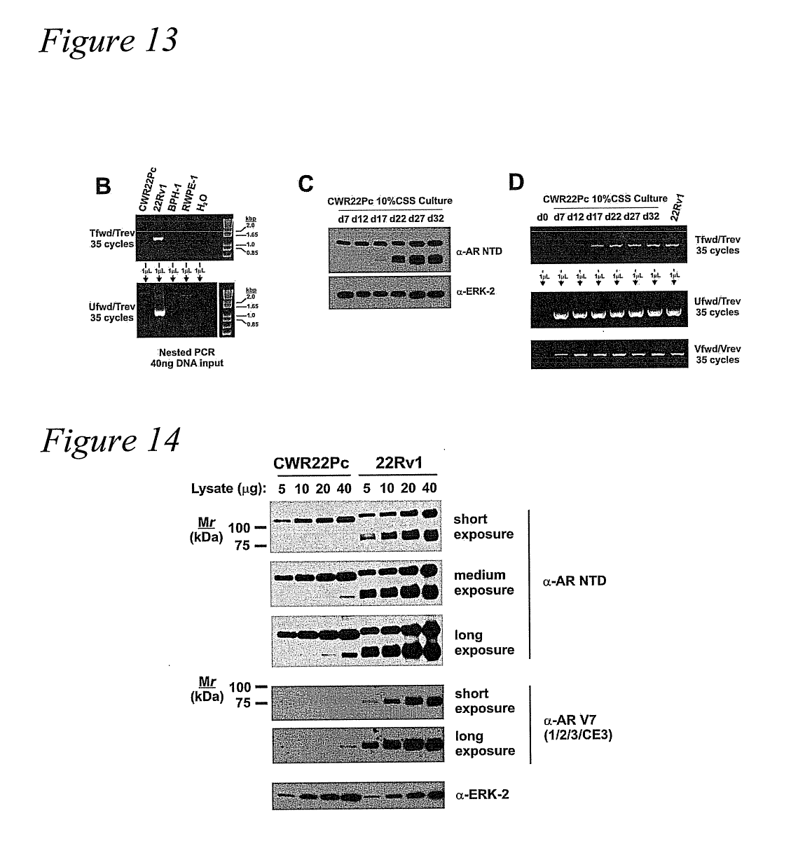

Using conventional and nested PCR strategies, we confirmed that the AR breakpoint observed in 22Rv1 cells was indeed restricted to this cell line (FIG. 21C and FIG. 13B). Previous studies have demonstrated that androgen-dependent CWR22Pc xenograft tumors initially regress during castration, but eventually recur with a CRPCa phenotype (Dagvadorj et al., 2008 Clin Cancer Res 14:6062-72). To probe the link between AR intragenic rearrangement and CRPCa, we cultured CWR22Pc cells over a one-month period in androgen-depleted medium. During the first 12 days of culture, no changes in AR protein expression patterns were observed (FIG. 13C). Interestingly, the 22Rv1 breakpoint was detected in CWR22Pc cells by nested PCR after seven days of castration (FIG. 13D). The sensitivity of this nested PCR approach was determined to be as low as 1-2 genomes in limiting dilution assays (FIG. 21D), indicating that the sub-population of cells harboring this rearrangement was very rare. By day 17, discrete proliferative foci were apparent, which coincided with faint expression of truncated AR isoforms (FIG. 13C) and detection of the 22Rv1 breakpoint via conventional PCR (FIG. 13D). On day 17, cells were trypsinized and re-seeded to disperse these proliferative foci. By day 22 and onward, androgen-independent cell growth was apparent, as was the expression of truncated AR isoforms. Together, these findings demonstrate that AR intragenic rearrangement is linked to high-level truncated AR isoform expression and CRPCa growth in a cell-based model of PCa progression.

Recent reports describing the synthesis and function of truncated, constitutively active AR isoforms have provided a novel and conceptually simple mechanism for the resistance of CRPCa cells to androgen depletion (Dehm et al., 2008 Cancer Res 68:5469-77; Guo et al., 2009 Cancer Res 69:2305-13; Hu et al., 2009 Cancer Res 69:16-22; Sun et al., 2010 J Clin Invest 120:2715-30). However, the mere presence of truncated AR isoforms does not correlate perfectly with androgen responsiveness, which highlights the importance of quantitative understanding in this area. This is especially apparent from a recent study demonstrating that AR 1/2/3/CE3 (also termed AR-V7 (Hu et al., 2009 Cancer Res 69:16-22) or AR-3 (Guo et al., 2009 Cancer Res 69:2305-13)) increases during progression to CRPCa, but is also expressed in benign prostate tissue and hormone naive PCa, (Guo et al., 2009 Cancer Res 69:2305-13). Because truncated AR isoforms were originally identified in CRPCa cells derived from the CWR22 model, the recent establishment of an androgen-dependent cell line from CWR22 xenografts has permitted an evaluation of the changes in AR mRNA splicing regulation that may occur during PCa progression in a lineage-related context. One striking difference between androgen-dependent CWR22Pc cells and 22Rv1 CRPCa cells was the expression profile of full-length and alternatively-spliced AR mRNAs. Although alternatively spliced AR mRNAs and protein were detectable in both cell lines, we found that 22Rv1 cells had an enhanced capacity to efficiently synthesize AR 1/2/2b, AR 1/2/3/2b, and AR 1/2/3/CE3 mRNAs. We further demonstrated tandem duplication of a ˜35 kb segment harboring these alternative exons as a likely basis for the de-regulation of AR mRNA splicing observed in 22Rv1 cells. Moreover, two additional alternative exons expressed in VCaP cells are clustered on this segment between AR Exons CE1 and CE3 (Watson et al., 2010 Proc Natl Acad Sci USA, 107:16759-65). Mechanistically, such a rearrangement could impair normal splicing by lengthening of the already vast distance between the AR transcription start site and AR Exon 4, increasing the likelihood of incorporating one of the 2 sets of alternative exons preceding Exon 4, disrupting the normal genomic organization of cis-acting intronic and exonic splicing elements, or any combination of these possibilities. It will be important to elucidate a clear cause/effect mechanism, but technical limitations such as the size of the AR locus (˜180 kb) and even larger aberrant locus in 22Rv1 cells (˜215 kb) will have to be addressed.

AR overexpression is common in CRPCa, and AR gene amplification is thought to be a main driver of increased AR protein expression (Edwards et al., 2003 Br J Cancer 89:552-6; Linja et al., 2001 Cancer Res 61:3550-5). Most prior assessments of AR amplification in PCa tissues employed FISH, which lacks resolution and does not permit accurate copy number assessment along the length of the AR gene (Haapala et al., 2001 Lab Invest 81:1647-51; Leversha et al., 2009 Clin Cancer Res 15:2091-7; Linja et al., 2001 Cancer Res 61:3550-5; Bubendorf et al., 1999 Cancer Res 59:803-6; Visakorpi et al., 1995 Nat Genet 9:401-6). Our findings indicate that a subset of amplified AR loci in CRPCa harbor intragenic rearrangements similar to 22Rv1, in addition to other alterations, which would clearly lead to a reconfigured AR exon organization for many of these alleles. One possibility is that there is selection for intragenic rearrangement; alternatively, rearrangement may simply occur as a byproduct AR gene amplification.

Long-term castration of CWR22Pc cells suggests that AR intragenic rearrangement and the CRPCa growth phenotype are linked, and that enrichment for cells with this genomic alteration occurs because of a selective advantage under castrate conditions. It is also possible that larger-scale genomic rearrangements may play a role in disrupted AR splicing, as evidenced by the recent identification of the truncated mAR-V4 isoform in the Myc-CaP mouse model, which results from alternative splicing of a cryptic exon nearly 1 Mb upstream of the mouse AR locus (Watson et al., 2010 Proc Natl Acad Sci USA, 107:16759-65). Together, these findings indicate that tissues displaying AR intragenic rearrangements should be prioritized for further studies of AR splice variants and their importance to PCa prognosis and therapeutic response.

Our work has revealed LINE-1 elements at the 5′ and 3′ ends of the 22Rv1 AR rearrangement. Transposable elements (TEs) are implicated in the genesis of rearrangements underlying TE-related genetic diseases, including cancer (Belancio et al., 2008 Genome Res 18:343-58), and often arise through non-allelic homologous recombination (NAHR). However, sequencing the 22Rv1 AR break fusion junction revealed a 27 bp insertion of unknown origin, which opposes a NAHR-based model. Indeed, stressed cancer cells are deficient in NAHR (Bindra et al., 2007 Cancer Metastasis Rev 26:249-60) and cancer-specific rearrangements frequently contain insertions ranging from 1 bp to 154 bp of so-called non-template sequence at the break fusion junction (Bignell et al., 2007 Genome Res 17:1296-303; Campbell et al., 2008 Nat Genet 40:722-9; Stephens et al., 2009 Nature 462:1005-10). Therefore, a new model, termed microhomology-mediated break-induced replication (MMBIR) has recently been proposed to account for this class of break fusion junctions in cancer cells (Hastings et al., 2009 PLoS Genet 5:e1000327).

In summary, our work describes a novel AR intragenic rearrangement in the 22Rv1 model of PCa progression, which is linked to enhanced synthesis of truncated AR isoforms and androgen-independent growth. We further demonstrate that similar genomic rearrangements occur in metastatic CRPCa specimens. A genomic basis for pathologic AR isoform expression may serve as a stable mechanism-based marker for resistance to androgen depletion therapies.

Thus, rearrangements in the AR gene underlie stable and efficient synthesis of alternatively-spliced, truncated AR isoforms that can support the CRPCa phenotype.

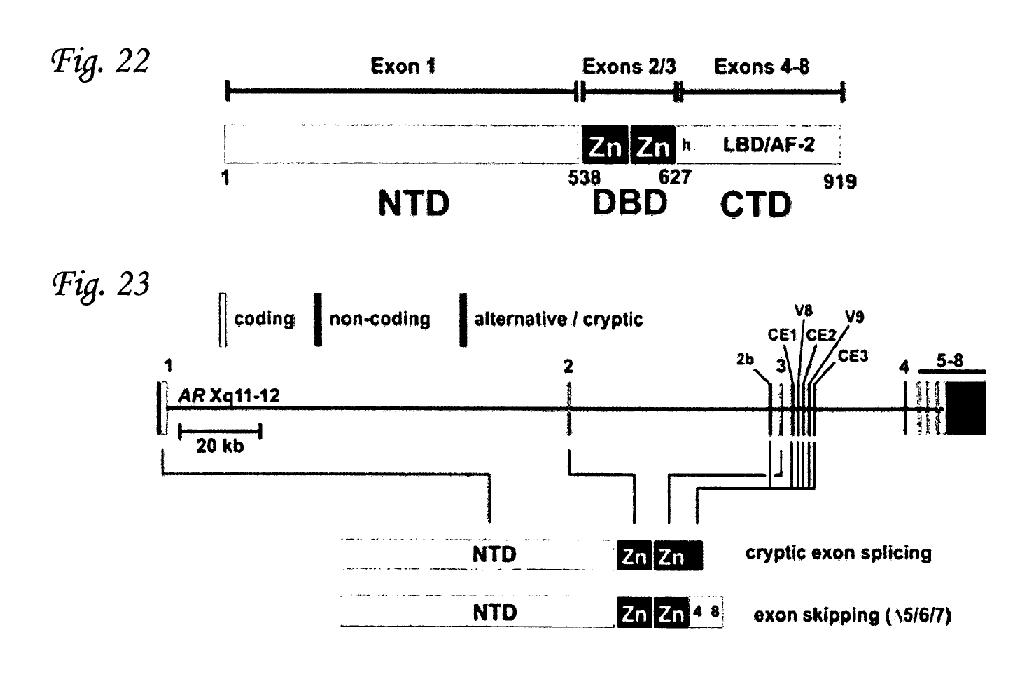

AR protein modularity is reflected by the organization of exons within the 180 kb AR locus at chromosome position Xq11-12 (FIG. 22). AR Exon 1 encodes the entire 538 amino acid AR NH2-terminal domain (NTD), which is structurally flexible and accounts for the majority of AR transcriptional activity (Layery and McEwan, 2005 Biochem J, 391(Pt 3):449-64; Layery and McEwan, 2006 Biochem Soc Trans 34(Pt 6):1054-7; Dehm and Tindall, 2007 Mol Endocrinol, 21(12):2855-63). Exons 2 and 3 each encode one of the two zinc fingers constituting the 89 amino acid DNA binding domain (DBD), and Exons 4-8 encode the 292 amino acid COOH-terminal domain (CTD) which harbors a short hinge region, the ligand binding domain (LBD), and transcriptional activation function-2 (AF-2) (Dehm and Tindall, 2007 Mol Endocrinol, 21(12):2855-63; Warnmark et al., 2003 Mol Endocrinol, 17(10):1901-9; He et al., 2004 Mol Cell, 16(3):425-38). To date, AR mutation analysis has been restricted to these coding sequences (Haapala et al., 2001 Lab Invest, 81(12):1647-51; Hyytinen et al., 2002 Lab Invest, 82(11):1591-8; Steinkamp et al., 2009 Cancer Res, 69(10):4434-42; Taplin et al., 1999 Cancer Res, 59(11):2511-5; Taplin et al., 1995 N Engl J Med, 332(21):1393-8; Taplin et al., 2003 J Clin Oncol, 21(14):2673-8; Thompson et al., 2003 Lab Invest, 83(12):1709-13; Tilley et al., 1990 Cancer Res, 50(17): p. 5382-6). The prevalence of AR mutations found in different studies varies, but appears to be low at approximately 10% (Haapala et al., 2001 Lab Invest, 81(12):1647-51; Hyytinen et al., 2002 Lab Invest, 82(11):1591-8; Steinkamp et al., 2009 Cancer Res, 69(10):4434-42; Taplin et al., 1999 Cancer Res, 59(11):2511-5; Taplin et al., 1995 N Engl J Med, 332(21):1393-8; Taplin et al., 2003 J Clin Oncol, 21(14):2673-8; Thompson et al., 2003 Lab Invest, 83(12):1709-13; Tilley et al., 1990 Cancer Res, 50(17): p. 5382-6) However, patients treated with antiandrogens, particularly flutamide, likely exhibit a higher rate (Steinkamp et al., 2009 Cancer Res, 69(10):4434-42; Taplin et al., 1999 Cancer Res, 59(11):2511-5). Functional studies of AR mutants have demonstrated altered ligand specificity, allowing inappropriate activation by alternative steroid ligands and antiandrogens (Culig et al., 1993 Mol Endocrinol, 7(12):1541-50; Culig et al., 1996 Cancer Detect Prey, 20(1):68-75; Zhao et al., 2000 Nat Med, 6(6):703-6), as well as transactivation in response to low levels of DHT (Buchanan et al., 2001 Mol Endocrinol, 15(1):46-56).

Prior to the AR isoforms described herein, most alternatively-spliced AR exons are clustered around AR Exon 3 (FIG. 23 and Dehm et al., 2008 Cancer Res, 68(13):5469-77; Guo et al., 2009 Cancer Res, 69(6):2305-13; Hu et al., 2009 Cancer Res, 69(1):16-22; Watson et al., 2010 Proc Natl Acad Sci USA, 107:16759-65). We describe herein, however, discovery of intragenic rearrangement involving this genomic segment as a mechanism for disrupted AR splicing in CRPCa cells. These data support a new genetic paradigm for AR variants in CRPCa, which predicts that structural alterations in the ˜180 kb AR gene underlie disrupted splicing and enhanced synthesis of constitutively active, truncated AR isoforms that contribute to ADT resistance.

With this new genetic paradigm of PCa progression in mind, our bioinformatics team devised a new computational algorithm for genomic segmentation using high-resolution genome-wide copy number data from PCa tissues. This approach identified focal copy number alterations involving AR Exon 3 and alternative exons in at least one metastasis from 6/14 (42.9%) rapid autopsy CRPCa subjects, but not in primary PCa (Example 2). Intriguingly, focal copy number alterations within the AR Exon 3 region were accompanied by additional AR copy number alterations as well as AR amplification, indicating a frequent and high degree of complexity in AR gene structure that has not been recognized previously. This is likely because AR gene analysis has traditionally been performed using low-resolution approaches such as FISH, and sequencing efforts have been restricted to AR exons, which represent only ˜1.5% of the AR gene. Therefore, one can define the sequence and structure of rearranged and amplified AR “alleles” in CRPCa using, for example, a novel high-throughput multiplexed assay to rapidly identify AR copy number imbalances, a paired-end next-generation sequencing workflow that can resolve the structure and sequence of focal AR gene rearrangements in CRPCa tissues, and, for example, an absolute quantification assay as well as next-generation RNA-Seq. Such analysis can reveal the impact of altered AR gene structure on AR splicing patterns.

These tools can lead to the development and clinical testing of new AR-targeted therapies for PCa, which can be evaluated using unique vectors and models of PCa progression described herein.

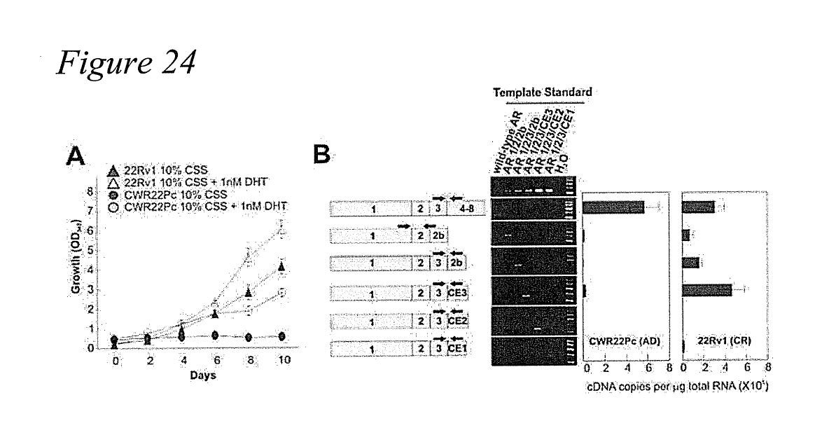

We have studied isogenic pairs of PCa cell lines to investigate the function of truncated AR isoforms during PCa progression. Both CWR22Pc and 22Rv1 cells were derived from the same CWR22 human PCa xenograft (Dagvadorj et al., 2008 Clin Cancer Res, 14(19):6062-72), but CWR22Pc cells are strictly androgen-dependent while 22Rv1 cells can grow with a CRPCa phenotype that is mediated by constitutive activity of truncated AR isoforms (FIG. 24A and Dam et al., 2008 Cancer Res, 68(13):5469-77; Guo et al., 2009 Cancer Res, 69(6):2305-13; Hu et al., 2009 Cancer Res, 69(1):16-22). Because analysis of the various alternatively-spliced AR mRNA isoforms by RT-PCR requires the use of different 3′ PCR primers with different amplification efficiencies, the traditional differential threshold cycle of amplification (2^{-ΔΔCt}) method of quantification is not appropriate. To overcome this technical hurdle, we developed an absolute quantification strategy using standard curves to derive copy number (FIG. 24B). Using this approach, high levels of full-length AR mRNA as well as the AR 1/2/2b, 1/2/312b, and AR 1/2/3/CE3 mRNAs were observed in 22Rv1 cells (FIG. 24B). In androgen-dependent CWR22Pc cells, full-length AR expression was predominant, but expression of AR 1/2/3/CE3 and AR 1/2/2b mRNA was also detectable (FIG. 24B and Example 2). These mRNA expression profiles were stable and matched the expression profile of full-length and truncated AR proteins in these cell lines (Example 2). These data demonstrate that both androgen-dependent CWR22Pc and CRPCa 22Rv1 cells can synthesize truncated AR isoforms at the mRNA and protein level, but 22Rv1 cells are able to sustain stable, high-level expression.

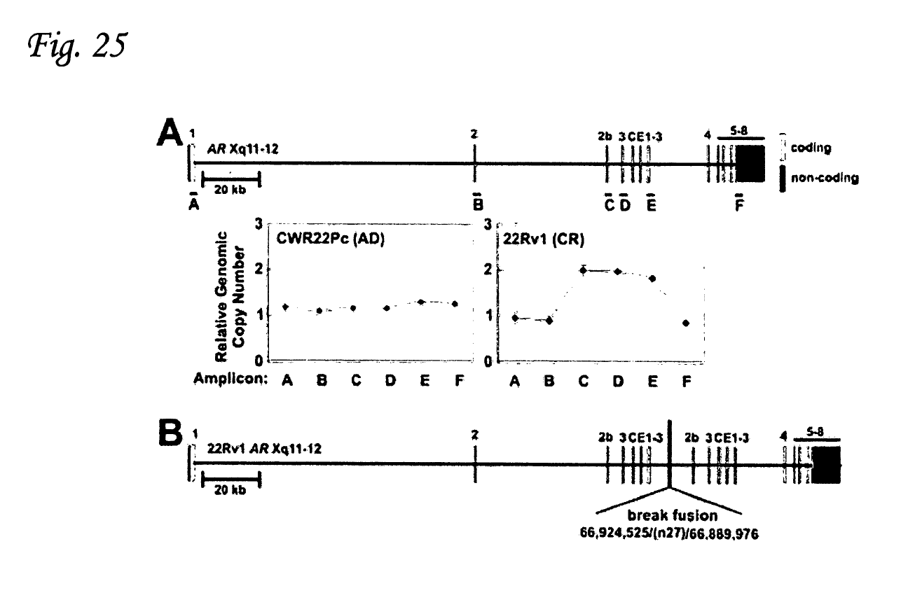

Because 22Rv1 cells can efficiently synthesize mRNAs with contiguously-spliced Exons 1, 2, 3, and 2b (FIGS. 23 and 24), and Exon 2b is situated upstream of Exon 3, a genomic aberration in the AR locus may underlie the stable splicing alterations observed in these cells. Indeed, in CRPCa 22Rv1 cells, we observed increased copy number of AR Exons 2b, 3, and CE3, confirming a rearrangement involving this genomic segment (FIG. 25A). Higher-resolution copy number interrogation as well as breakpoint PCR with outward-facing primers led to the cloning and characterization of this genomic 22Rv1 breakpoint, revealing tandem duplication of a ˜35 kb segment encompassing AR Exon 3 and alternative exons (FIG. 25B and Example 2). This finding defines a novel class of genomic aberration involving the AR locus in CRPCa cells.

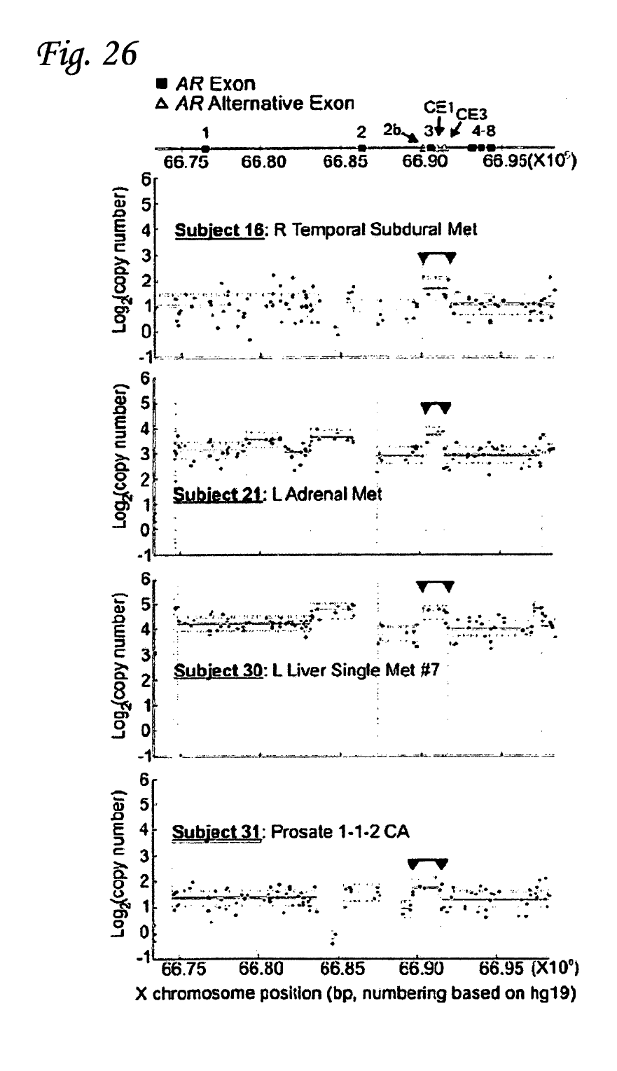

AR gene alterations in PCa have been studied for over a decade, and have revealed point mutations and amplification as important mechanisms for CRPCa. Intragenic rearrangements such as those observed in 22Rv1 cells would not have been identified previously because sequencing has been restricted to AR exons or cDNAs (Haapala et al., 2001 Lab Invest, 81(12):1647-51; Hyytinen et al., 2002 Lab Invest, 82(11):1591-8; Steinkamp et al., 2009 Cancer Res, 69(10):4434-42; Taplin et al., 1999 Cancer Res, 59(11):2511-5; Taplin et al., 1995 N Engl J Med, 332(21):1393-8; Taplin et al., 2003 J Clin Oncol, 21(14):2673-8; Thompson et al., 2003 Lab Invest, 83(12):1709-13; Tilley et al., 1990 Cancer Res, 50(17): p. 5382-6), and AR FISH with large BAC probes does not have the resolution to identify structural alterations of this scale (Edwards et al., 2003 Br J Cancer, 89(3):552-6; Linja et al., 2001 Cancer Res, 61(9):3550-5; Ford et al., 2003 J Urol, 170(5):1817-21; Visakorpi et al., 1995 Nat Genet, 9(4):401-6; Liu et al., 2009 Nat Med, 15(5):559-65). Therefore, to determine whether related AR gene structural alterations occur in clinical PCa, we developed a computational algorithm for segmentation analysis of high-resolution Affymetrix Genome-Wide Human SNP Array 6.0 (SNP6.0) data derived from clinical primary PCa (44 tumors from 44 patients) and metastatic CRPCa (58 metastases from 14 patients) (Liu et al., 2009 Nat Med, 15(5):559-65; Mao et al., 2010 Cancer Res, 70(13):5207-12). The algorithm is dynamic and designed to estimate the number and locations of segments adaptively based on probe-level data. Using this approach, focal copy number increases were observed between AR Exons 2/3 and 3/4 in 12/58 (20.7%) CRPCa metastases from 6/14 (42.9%) rapid autopsy subjects, which presented as rearrangement of a segment encompassing AR Exon 3 and alternative exons (FIG. 26). Copy number alterations involving this genomic segment were consistently observed concurrent with AR gene amplification in CRPCa. We confirmed focal copy number increase in the region between AR Exons 2 and 4 in two separate metastases from a single rapid autopsy subject using our PCR-based copy number assay (Example 2). SNP6.0 analysis identified additional breakpoints within the AR locus in most CRPCa samples, which indicates even more complexity in the architecture of amplified AR alleles than anticipated (FIG. 26). This concept of amplified and rearranged AR loci in CRPCa is further supported by truncated mouse AR variant 4 (mAR-V4), which results from splicing of mouse AR Exons 1, 2, 3, 4, and a cryptic exon located 1 Mbp upstream of the amplified AR gene in Hi-Myc mouse PCa cells (Watson et al., 2010 Proc Natl Acad Sci USA, 107:16759-65).

Overall, these data identify AR intragenic rearrangement as a novel genetic aberration that alters AR gene architecture in CRPCa. AR intragenic rearrangements can correlate with disrupted AR splicing patterns in CRPCa.

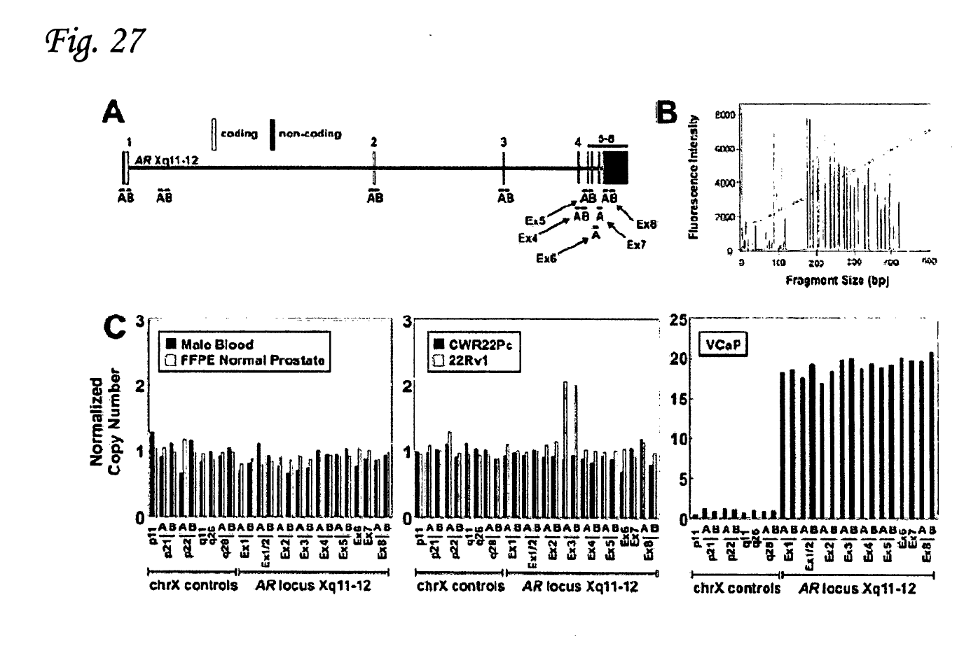

The qPCR-based copy number assay we developed was effective for discovering AR intragenic rearrangements, but may not be optimal for use with large numbers of clinical samples (FIG. 25A). Moreover, the accuracy of qPCR can decrease somewhat with increasing gene copy number (Perne et al., 2009 Biotechniques, 47(6):1023-8) and our data indicate that AR loci harboring AR intragenic rearrangements also may be amplified (FIG. 26). However, multiplex ligation-dependent probe assay (MLPA) may be used to determine AR copy number in clinical PCa specimens. This is a high-throughput, multiplexed assay that measures AR copy number at multiple loci (FIG. 27A) in a single tube using as little as 10 ng of input DNA (den Dunnen and White, “MLPA and MAPH: sensitive detection of deletions and duplications,” in Current Protocols in Human Genetics, 2006. Chapter 7: p. Unit 7.14). Multiplexing is based on the use of variable-length 2-part probes, which hybridize to target regions and can be ligated and amplified with labeled universal 5′ and 3′ primers. The variable-length products are resolved by capillary electrophoresis (FIG. 27B). Peak areas are normalized to derive copy number. We have verified this approach with various input samples, including normal male blood, formalin-fixed, paraffin-embedded (FFPE) PCa tissue, CWR22Pc/22Rv1 cells, as well as androgen-dependent VCaP cells, which display uniform AR gene amplification (FIG. 27C). One caveat is that this commercially-available AR MLPA kit (MRC Holland; Amsterdam, the Netherlands) mainly interrogates copy number at AR Exons (FIG. 27A), whereas we identify focal copy number changes involving both coding and non-coding sequence. One can, however, use a commercially-available probe-free, reagent-only MLPA kit (MRC Holland; Amsterdam, the Netherlands), and create a second AR Intron-focused MLPA assay. To this end, one can add the same X-chromosome control probes that are used in the exon-focused AR MLPA kit, and design and optimize a set of 16 additional probes that interrogate copy number between AR Exons 1 and 4. AR Exon- and Intron-focused MLPA kits can be used to assess AR gene copy number in, for example, 170 androgen-dependent PCa and, for example, 81 CRPCa tissue specimens of local and metastatic disease shown in Table 5.

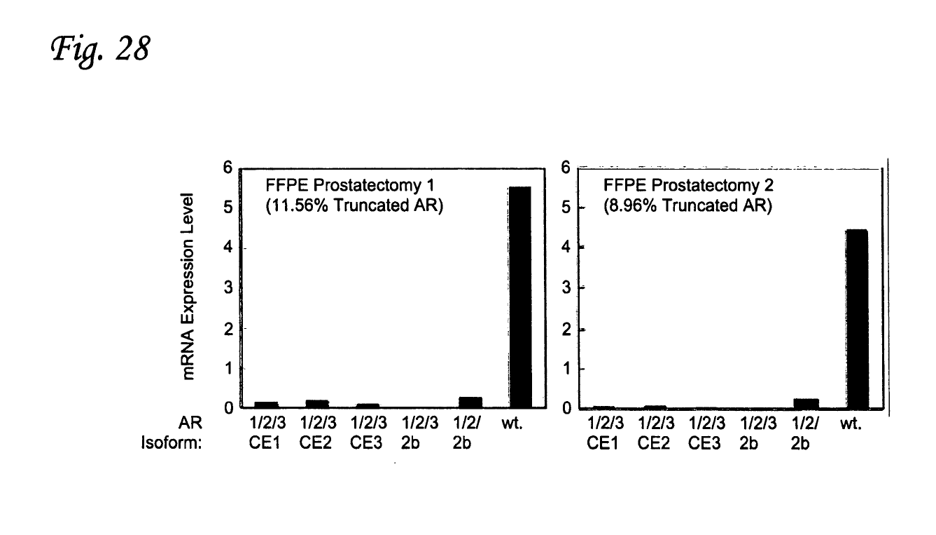

One can also assess AR copy number in, for example, 24 PCa xenografts, many of which are isogenic androgen-dependent/CRPCa pairs that express increased levels of alternatively-spliced truncated AR isoforms during progression (Dam et al., 2008 Cancer Res, 68(13):5469-77; Hu et al., 2009 Cancer Res, 69(1):16-22; Sun et al., 2010 J Clin Invest, 120(8):2715-30). One also can employ targeted RT-PCR with these tissue specimens to determine whether focal changes in AR gene copy number correlate with enhanced synthesis of known alternatively-spliced, truncated AR isoforms. We have verified that our absolute quantification assay is suitable for FFPE tissue, using RNA isolated from 1 mm×0.5 mm tissue discs punched from 10-year-old prostatectomy blocks. Agilent Bioanalyzer analysis indicating that the RNA was highly degraded, yet RT-PCR analysis resulted in detection and absolute quantification of full-length and truncated AR mRNA isoforms (FIG. 28).

TABLE 5
PCA Tissue Specimens

| | Number | Source | Storage |
|---|---|---|---|
| Androgen Dependent, | 170^{a} | prostatecomy | FFPE^{e} |
| Primary | | | |
| Castration Resistant, | 40^{b} | TURP^{d} | FFPE^{e} |
| Local Recurrence | | | |
| Castration Resistant, | 26^{a} | biopsy | FFPE^{e} |
| Metastasis | | | |
| Castration Resistant, | 15^{c} | rapid autopsy | fresh frozen |
| Metastasis | | | |
| PCa Xenografts, variable | 24^{c} | mouse xenograft | fresh frozen |
| androgen-dependence | | | |

^{a}Dr. Schmechel, U of MN BioNET;
^{b}Dr. Bismar, U of Calgary;
^{c}Dr. Vessella, U of Washington
^{d}TURP, transurethral resection of the prostate;
^{e}FFPE, formalin-fixed, paraffin embedded

Truncated AR isoform expression can be higher in CRPCa versus androgen-dependent PCa. Quantitative AR mRNA expression data can be analyzed using unpaired T-tests or Mann-Whitney test. Also, a subset of CRPCa specimens can display changes in the ratio of alternatively-spliced AR relative to wild-type AR, and this can correspond to those CRPCa specimens that display quantitative copy number imbalances within the AR locus, including the Exon 3 region. To test for this association, one can perform Pearson or Spearman correlation or regression (nonlinear or logistic) analysis.

### Next-Generation Genomic DNA and RNA Sequencing in Clinical CRPCa Metastases.

The genomic analysis described immediately above measures copy number, but may not identify the sequence or architecture of rearranged segments in the AR locus. To understand how AR gene structure alterations may impact AR splicing and expression patterns, one can define the sequence, structure, and/or coding capacity of the various AR “alleles” that arise in CRPCa. To this end, one can sequence and assemble the entire AR gene as well as sequence resulting AR mRNAs in CRPCa metastasis and PCa xenografts.

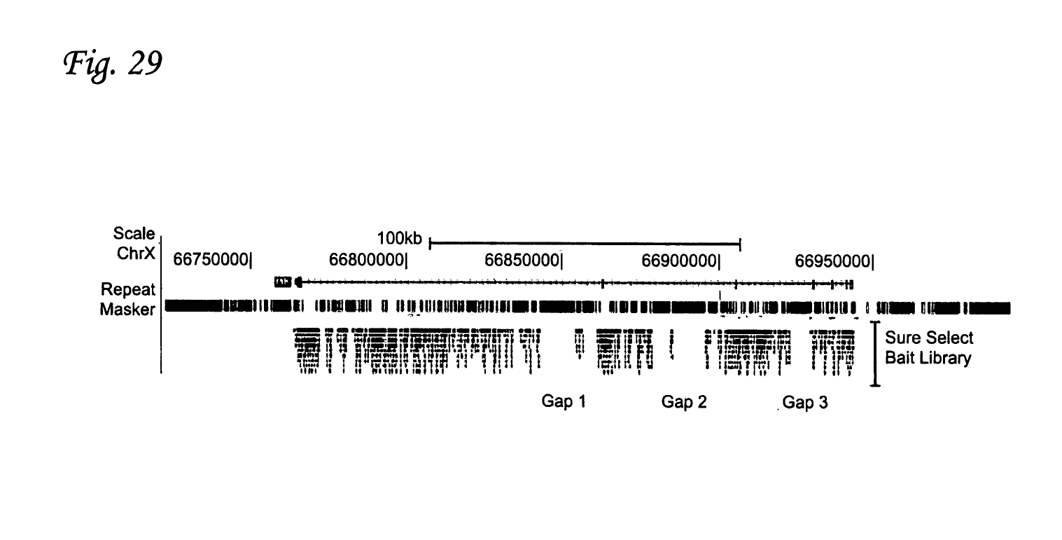

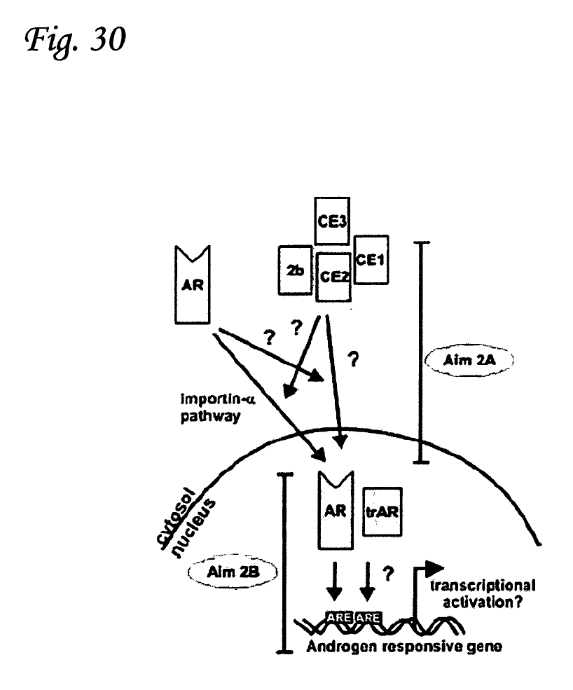

One can use an approach that includes, for example, sequence capture and paired end sequencing. Genomic DNA from, for example, 15 rapid autopsy CRPCa metastases and, for example, 24 mouse xenografts can be sheared and size-selected to a uniform size of, for example, either 2 kb or 20 kb. The fragments may be used to generate two sets of paired-end sequencing libraries using commercially available kits and protocols. Library fragments representing the AR locus can be isolated using a custom-designed Agilent SureSelect liquid-phase bait capture library (FIG. 29), and 75 by paired-end reads can be obtained using an Illumina GAIIx sequencer. Captured genomic DNA library fragments can be sequenced in pools of, for example, 12 samples, which is a common maximum number of barcodes available in commercial paired-end sequencing kits. Assuming 90% SureSelect enrichment, 12 pooled barcoded samples, and a total of 80×10⁶ individual reads of 150 bp (75 bp paired-end reads) per sequencing run, this can result in 5,000× coverage of the ˜180 kb AR gene per sample.

One can also perform high-throughput RNA sequencing on AR mRNAs in these metastases and xenografts. To this end, total RNA can be reverse transcribed to cDNA and fragmented. Size-selected fragments can be used to generate paired-end sequencing libraries. Library fragments originating from the AR locus can be isolated using, for example, the same custom-designed Agilent SureSelect liquid-phase capture kit that can be used for genome sequencing (FIG. 29). Assuming 90% enrichment using SureSelect and a total of 80×10⁶ individual reads of 150 bp (75 bp paired-end reads) with the GAIIx instrument, this can result in 300,000× coverage of the ˜3 kb full-length AR cDNA per sample.

Analysis of paired-end reads using the Cuflinks algorithm (Trapnell et al., 2010 Nat Biotechnol, 28(5):511-5) can identify alternative splicing events quantitatively. Any frequent alternative splicing events discovered using this approach can be verified with targeted studies as described herein. Altered AR splicing patterns can occur in tissues displaying AR gene rearrangements. Paired end sequencing technology has been superior to single-read sequencing for reducing ambiguities in mapping to reference genomes as well as identifying remote translocations/gene fusions and local medium-scale rearrangements (Quinlan et al., 2010 Genome Res. 20(5):623-35). Based on the 22Rv1 AR rearrangement and SNP6.0 analysis of the AR locus in CRPCa cells (FIGS. 25 and 26), we have designed an algorithm for identifying rearrangements and assembling paired-end reads into intact AR alleles. This strategy can be implemented and optimized with paired-end reads from CWR22Pc and 22Rv1 genomic DNA to “re-discover” the tandem duplication shown in FIG. 25B de novo. First, one can map all reads to the reference human genome using the highly efficient and accurate algorithms bowtie or BWA (Langmead et al., 2009 Genome Biol, 10(3):R25; Li and Durbin, 2009 Bioinformatics 25(14):1754-60) and compute depth of coverage ratios relative to a reference sample of pooled normal male DNA. Mapped read pairs can be classified as normal (mapping distance within 2 standard deviations of expected fragment size and reads with expected inward-facing orientation), or aberrant (unmappable, singleton-mapped, stretched, shortened, and/or aberrant outward/forward/reverse-facing orientation). Candidate rearrangement units including inversions, tandem repeats, deletions, and insertions, can be identified using heuristic criteria based on discriminating sequence properties of each of these units. One can also, for example, require at least two corroborating aberrant reads for each rearrangement identified. A fictitious altered genomic reference sequence can then be created by “undoing” each rearrangement with approximated breakpoints identified from zeroes in coverage ratio plots. Breakpoint regions for tandem repeats can also be determined from these plots at the positions where average coverage ratios jump to a higher integral value. More precise breakpoints can be identified using the unmappable reads with an algorithm similar to that used by TopHat (Trapnell et al., 2009 Bioinformatics, 25(9):1105-11), wherein 75 bp reads can be split into subfragments (e.g., 3 subfragments of 25 bp each). Two of these subfragments should map properly, anchoring the overall mapping, and all possible sub alignments of the intervening region can be indexed (taking into consideration permutations of hypothetical inversions or repeats) for quick identification of the actual breakpoint. Once precise breakpoints are identified, computational inversions, tandem repeats, and deletions can be carried out on the reference genome directly. Novel insertions can be reconstructed by assembling the unmappable reads using Velvet (Zerbino and Birney, 2008 Genome Res, 18(5):821-9), and picking out those contigs that are flanked on either end by singleton-mapped reads whose reference-mapped ends join up with the reference sequence flanking the insert on both sides. At the end of this procedure, the original reads from a particular sample can be mapped back to the newly rearranged “reference” locus to ensure that unmappable reads and read mapping aberrations are minimized.

A scenario of genomic rearrangements involving the AR locus in CRPCa leading to disrupted splicing patterns may indicate that new truncated AR isoforms with different COOH-terminal extensions can be identified, thus adding to a growing list of proteins (Dehm et al., 2008 Cancer Res, 68(13):5469-77; Guo et al., 2009 Cancer Res, 69(6):2305-13; Hu et al., 2009 Cancer Res, 69(1):16-22; Sun et al., 2010 J Clin Invest, 120(8):2715-30; Watson et al., 2010 Proc Natl Acad Sci USA, 107:16759-65) that are nearly identical in structure and sequence (FIG. 23). Therefore, one can establish a set of “facts” or “rules” regarding the similarities or differences in the biochemical properties of these truncated AR isoforms. For example, the unique COOH-terminal extensions of individual truncated AR isoforms may influence modulating activity due to their proximity to the AR DBD (Guo et al., 2009 Cancer Res, 69(6):2305-13; Sun et al., 2010 J Clin Invest, 120(8):2715-30). Moreover, truncated AR isoforms may exert their cellular activities in part through interactions with wild-type AR, which may be relevant because truncated and wild-type AR isoforms are often co-expressed (Dehm et al., 2008 Cancer Res, 68(13):5469-77; Guo et al., 2009 Cancer Res, 69(6):2305-13; Hu et al., 2009 Cancer Res, 69(1):16-22; Sun et al., 2010 J Clin Invest, 120(8):2715-30; Watson et al., 2010 Proc Nati Acad Sci USA, 107:16759-65). Determining the mechanisms by which certain steps in transcriptional activation are regulated may identify new PCa therapeutic targets and reveal mechanisms for any specific differences that may be mediated by divergent COOH-terminal extensions.

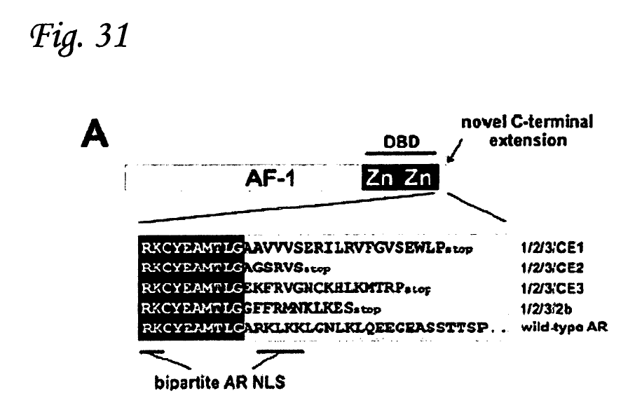

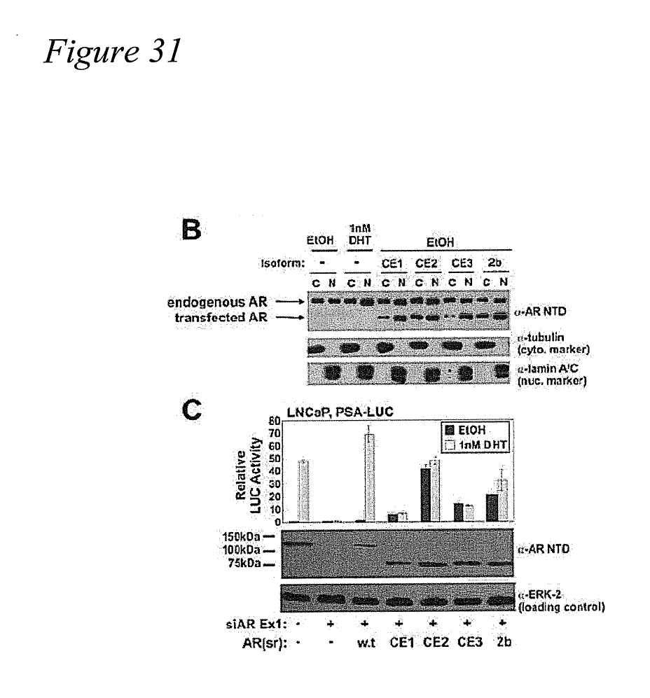

Truncated AR isoforms with divergent COOH-terminal extensions can access the nucleus, albeit to varying degrees (FIG. 31A and FIG. 31B). It is not entirely clear how truncated AR isoforms can access the nucleus, because alternative splicing of cryptic exons disrupts the well characterized bipartite AR nuclear localization signal (NLS) RKcyeamtlgaRKLKK (SEQ ID NO: 102), which interacts directly with importin-α (Black and Paschal, 2004 Trends Endocrinol Metab, 15(9):411-7). However, it is possible that cryptic exon-encoded amino acids can reconstitute a functional bipartite NLS. For example, exons CE3 and 2b encode KHLK and KLK motifs, respectively (FIG. 31A). Because the AR 1/2/3/4/8 truncated AR isoform retains the intact AR NLS (Sun et al., 2010 J Clin Invest, 120(8):2715-30), this isoform can also be included in these studies.

To evaluate the impact of specific COOH-terminal residues on nuclear localization in more detail, one can take a deletion and site-directed mutagenesis approach. For example, first, one can generate an AR 1/2/3 mutant with a stop codon immediately following the AR Exon 3-encoded CYEAMTLG (SEQ ID NO: 103) (FIG. 10A). One also can generate versions of truncated ARs with RK to AA mutations, which can disrupt the first half of the bipartite NLS. For these experiments, one can transfect DU145 (AR-null) and LNCaP (AR-dependent) PCa cells with wild-type and mutant constructs. One also can use, for example, COS-7 cells to confirm salient findings because much of the steroid receptor localization literature is based on the use of these cells. For localization experiments in LNCaP cells, one can use N-terminal hemagglutinin (HA) epitope-tagged versions of all constructs in order to discriminate between ectopic and endogenous AR proteins. Nuclear localization can be assessed using biochemical fractionation and confocal microscopy.

The AR 1/2/3/4/8 isoform can physically interact with full-length AR and thereby enhance nuclear localization of the un-liganded receptor (Sun et al., 2010 J Clin Invest, 120(8):2715-30). The effects of other AR isoforms on localization of full-length AR (and vice versa) have not been addressed. To this end, one can exploit siRNA-resistant versions of truncated AR isoforms we have developed (FIG. 31C). Localization of tagged, siRNA-resistant versions of truncated AR isoforms can be tested in LNCaP cells co-transfected with control siRNA (full-length AR expressed) or Exon 1-targeted siRNA (all endogenous AR expression knocked down). For biochemical fractionation experiments, Western blots can be probed with a “pan-AR” antibody that can detect all AR isoforms by virtue of recognizing the AR NTD (FIG. 31B and FIG. 31C). For confocal microscopy, detection of truncated and full-length AR isoforms can be achieved using either HA-specific or AR CTD-specific antibodies, respectively. Salient findings generated in LNCaP cells can be confirmed in DU145 and COS-7 cells by co-expressing wild-type and truncated versions of the AR. If full-length AR and truncated AR isoforms are deemed to influence each others' localization, one can examine the mechanism of this association using various mutant versions of full-length and truncated AR isoforms. These studies can be carried out using co-immunoprecipitation and GST pull-down experiments with an initial focus on the second zinc finger in the AR DBD, which encodes the AR dimerization interface (Shaffer et al., 2004 Proc Natl Acad Sci USA, 101(14):4758-63). Co-immunoprecipitation experiments also can be performed following depletion of DNA with ethidium bromide (Yu et al., 2010 Cancer Cell, 17(5):443-54) to determine whether interactions are DNA-dependent.

AR import proceeds through a mechanism in which ligand binding induces a conformational change in the AR LBD, which exposes the bipartite AR NLS and allows interaction with importin-α, leading to transport through the nuclear pore complex (Black and Paschal, 2004 Trends Endocrinol Metab, 15(9):411-7; Cutress et al., 2008 J Cell Sci, 121(Pt 7):957-68). One can evaluate the extent to which truncated AR isoforms use this import pathway by testing interactions between wild-type and mutant versions of truncated AR isoforms and endogenous importin-α in transfected cells using, for example, a combination of co-immunoprecipitation and confocal microscopy co-localization with AR- and importin-α antibodies as described (Cutress et al., 2008 J Cell Sci, 121(Pt 7):957-68; Kaku et al., 2008 Endocrinology, 149(8):3960-9). One can follow up with additional mutagenesis to evaluate how the second half of the bipartite AR NLS may be “reconstituted” by novel COOH-terminal extensions encoded by cryptic exons. The locations and identities of specific amino acid substitutions can be guided by the recently solved crystal structure of the AR DBD/LBD fragment in complex with importin-α (Cutress et al., 2008 J Cell Sci, 121(Pt 7):957-68).

Confocal microscopy data can be quantified by scoring at least 100 cells as primarily nuclear, primarily cytoplasmic, or equally nuclear and cytoplasmic. Student's T-tests can be used to evaluate quantitative differences in these studies. Based on our data showing that the AR 1/2/3/CE2 isoform can access the nucleus and activate transcription despite an apparent bipartite NLS (FIG. 31), the exact composition of the various COOH-termini may only modestly impact nuclear localization. Truncated AR isoforms can have strong constitutive transcriptional activity in the absence of full-length AR (FIG. 31C and Dehm et al., 2008 Cancer Res, 68(13):5469-77; Guo et al., 2009 Cancer Res, 69(6):2305-13); therefore, the full-length receptor may not necessarily be required for the localization of truncated AR isoforms. However, based on data showing that the truncated AR 1/2/3/4/8 isoform can enhance nuclear import of the full-length AR (Sun et al., 2010 J Clin Invest, 120(8):2715-30), truncated AR isoforms can influence nuclear localization of the full-length AR.

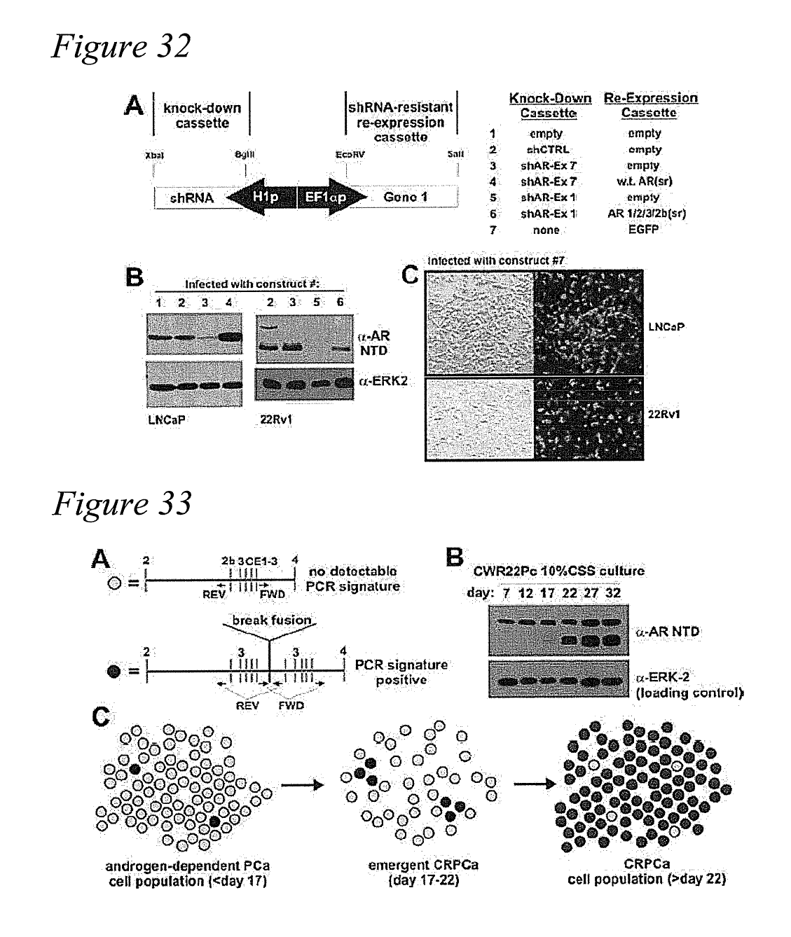

FIG. 31C shows that truncated AR isoforms can activate transcription of AR-responsive reporter genes in a ligand-independent fashion. The level of transcriptional activity may vary depending on cell line and promoter context, and may not correlate directly with nuclear localization (Dehm et al., 2008 Cancer Res, 68(13):5469-77; Guo et al., 2009 Cancer Res, 69(6):2305-13; Hu et al., 2009 Cancer Res, 69(1):16-22; Sun et al., 2010 J Chin Invest, 120(8):2715-30; Watson et al., 2010 Proc Nath Acad Sci USA, 107:16759-65; and FIG. 31). Because reporter gene assays may not recapitulate the complex chromatin organization or regulation of endogenous gene transcription, one may wish to determine whether truncated AR isoforms can access chromatin and activate endogenous gene transcription, and whether the various COOH-terminal extensions modulate these steps. To this end, we have generated a novel bidirectional lentivirus-based expression system to allow flexible knock-down and/or re-expression of snRNA-resistant wild-type and mutant versions of the AR in infected cells (FIG. 32). One strength of this system is that transduction efficiency can be consistently >95% and can remain stable at this level following more than a month of in vitro culture. One can first use this system to infect LNCaP and LAPC4 cells with either lentivirus that express truncated AR isoforms under control of the EF1α promoter or empty control virus. AR isoforms can be tagged at the N-terminus with a FLAG epitope to facilitate downstream molecular analysis. Infected cells can be treated with 1 nM DHT or maintained under castrate conditions. Expression of a panel of, for example, 12 androgen-regulated genes can be assessed by quantitative RT-PCR. Exemplary androgen-regulated genes include AR targets such as, for example, PSA, hK2, TMPRSS2, FKBP52, SCAP, and NKX3.1, but also genes identified (and validated) by genome-wide approaches as being important for PCa cell cycle regulation during prostate cancer progression (Dehm et al., 2008 Cancer Res, 68(13):5469-77; Dehm et al., 2007 Cancer Res, 67(20):10067-77; Dehm and Tindall, 2006 J Cell Biochem, 99(2):333-44; Ngan et al., 2009 Oncogene, 28(19):2051-63; Wang et al., 2009 Cell, 138(2):245-56). One can also perform chromatin immunoprecipitation (ChIP) to assess AR binding to relevant promoter and/or enhancer regions of these target genes, using anti-FLAG antibodies and primers for quantitative PCR that have been established in the literature (Wang et al., 2009 Cell, 138(2):245-56; Shang et al., 2002 Mol Cell, 9(3):601-10; Wang et al., 2005 Mol Cell, 19(5):631-42). These evaluations can identify common targets of truncated AR isoforms, but also can reveal differences in chromatin binding and/or transcriptional activity. One can also confirm important ChIP findings by testing endogenous AR 1/2/3/CE3 binding in 22Rv1 cells using an antibody specific for this isoform that we have used (Example 2). Where differences in target gene binding or activation by individual isoforms are observed, one can use COOH-terminal mutants generated as described herein to interrogate the role of specific amino acid sequences in these processes.

To test for functional associations between truncated AR isoforms and full-length AR, one can perform a similar set of experiments with the inclusion of AR-targeted or control shRNAs expressed from the histone H1 promoter in the bidirectional lentiviral vector. This can allow one to test whether the presence/absence of endogenous AR affects activity of ectopic snRNA-resistant AR isoforms. Where one obtains evidence for interactions between wild-type and truncated AR isoforms, one can use the bidirectional lentivirus system to express deletion and point mutant versions of truncated AR isoforms in LNCaP and LAPC4 cells to identify domains responsible for functional interaction with full-length AR, using, for example, qRT-PCR and ChIP as primary readouts. Similarly, one can knock-down full-length AR expression in 22Rv1 cells using shRNA targeted to AR Exon 7, and re-express deletion and point mutant versions of wild-type AR. The co-IP and co-localization approaches discussed herein can complement these RT-PCR and ChIP experiments.

Student's T-tests can be used to evaluate quantitative differences in chromatin binding and target gene activation in these studies. These studies can reveal differences in the sets of genes bound and activated by wild-type AR and individual truncated AR isoforms. However, the various truncated AR isoforms may activate similar target genes. Also, interactions may be observed between wild-type AR and truncated AR isoforms at different gene promoters, which may indicate heterodimerization.

Our data indicates that the CWR22Pc cell line has a strict androgen-dependent growth phenotype modulated by full-length AR (FIG. 24). However, as is the case for clinical disease, established CWR22Pc xenograft tumors can initially regress during castration, but eventually may recur with a CRPCa phenotype (Dagvadorj et al., 2008 Clin Cancer Res, 14(19):6062-72). Indeed, during one-month of maintenance under castrate conditions in vitro, the CWR22Pc cell line eventually assumes a CRPCa growth phenotype, which is marked by increased expression of truncated AR isoforms and enrichment of a rare sub-population of cells with the 22Rv1 break fusion junction signature (FIG. 33). These data demonstrate that AR intragenic rearrangement is linked to high-level truncated AR isoform expression and CRPCa growth in this novel model of PCa progression. To our knowledge, there are no other models of PCa progression where a measurable genomic marker of the CRPCa phenotype in an otherwise androgen-dependent PCa cell population is known a priori.

A fundamental theory in cancer biology is that targeted therapies (e.g. ADT) exert selective pressure that favors the emergence of rare tumor cell sub-populations with advantageous changes in the target (e.g. the AR). Our data suggests that a rare sub-population of CWR22Pc cells have a selective growth advantage under castrate conditions due to genomic rearrangement and efficient synthesis of truncated AR isoforms (FIG. 33). To characterize this model in greater detail, one can culture CWR22Pc cells in vitro under castrate versus 1 nM DHT conditions over a 30-day period, and measure various molecular and cellular parameters every five days. Emergence of the 22Rv1 break fusion junction can be monitored via breakpoint PCR (FIG. 33A). Truncated AR isoform expression can be assessed by, for example, Western blot as well as immunofluorescence using an antibody specific for the AR 1/2/3/CE3 isoform. One can also monitor the expression of an AR-regulated gene panel by, for example, quantitative RT-PCR. The CWR22Pc cell line can be highly tumorigenic in castrated mice supplemented with sustained-release DHT pellets, but may not form tumors in castrated mice (Dagvadorj et al., 2008 Clin Cancer Res, 14(19):6062-72). Therefore, to obtain a robust measure of the CRPCa phenotype for the overall cell population, one can, for example, collect cells every five days during this 30-day time-course and assess tumor-forming ability in, for example, athymic (Foxn1nu) mice castrated two weeks prior to tumor cell implantation (which can allow serum androgens to nadir). For these experiments, one can inject cells subcutaneously in each flank of, for example, six mice per time point as described (Dagvadorj et al., 2008 Clin Cancer Res, 14(19):6062-72), for a total of 82 mice (42 castrated, 42 castrated+DHT). These animal numbers can yield the requisite statistical power, which has been demonstrated by previous studies with similar models (Guo et al., 2009 Cancer Res, 69(6):2305-13; Corey et al., 2003 Prostate, 56(2):110-4; Sramkoski et al., 1999 In Vitro Cell Dev Biol Anim, 35(7):403-9). Tumor establishment and growth can be monitored with calipers, and animals can be euthanized if/when tumors reach a predetermined size such as, for example, 800 mm³. The time to establish tumors of the predetermined size can be recorded. All tumors can be removed, weighed, measured and frozen. Intratumoral AR expression can be assessed by Western blot and immunohistochemistry with pan-AR and AR 1/2/3/CE3 antibodies, and intratumoral AR function can be tested by measuring expression of an AR-regulated gene panel by quantitative RT-PCR.

One can further use lentivirus to express AR 1/2/3/2b or AR 1/2/3/CE3 under control of an EF1α promoter in CWR22Pc cells, which can induce androgen-independent AR activity and growth in infected cells (Guo et al., 2009 Cancer Res, 69(6):2305-13 Watson et al., 2010 Proc Natl Acad Sci USA, 107:16759-65). Empty lentivirus can be used as a control. Infected cells can be cultured under castrate versus 1 nM DHT conditions for 30 days, and subjected to the same molecular and cellular assays that can be used for model characterization. For this set of studies, in vivo xenograft experiments may be performed at, for example, only the Day 5 or Day 10 time point to establish whether transgene expression induces a CRPCa growth phenotype independent of the 22Rv1 breakpoint signature de novo.

One also can co-infect CWR22Pc cells with separate lentivirus vectors encoding shRNAs targeted to AR Exon 2b and CE3, which can elicit truncated AR isoform-specific knock-down (Dehm et al., 2008 Cancer Res, 68(13):5469-77; Guo et al., 2009 Cancer Res, 69(6):2305-13). Lentivirus expressing non-targeted shRNA can be used as a control. Infected cells can be cultured under castrate versus 1 nM DHT conditions for 30 days, and subjected to the same molecular and cellular assays that can be used for model characterization. For this set of studies, in vivo xenograft experiments may be performed at, for example, only the Day 25 or Day 30 time point to establish whether isoform knock-down has prevented emergence of a CRPCa population enriched for the 22Rv1 breakpoint signature.

Student's T-tests can be used to evaluate quantitative differences in all molecular and cellular parameters between experimental and control groups. Changes in cellular and molecular parameters during in vitro castration can be analyzed by ANOVA. Increasing duration of in vitro castration can result in a population with increased capacity to form tumors in castrated mice. Emergence of molecular features such as truncated AR expression and the 22Rv1 break fusion junction signature may not be observed in cells maintained in culture with 1 nM DHT, and these cells may not form tumors in castrated mice. Ectopic expression of AR 1/2/3/2b or AR 1/2/3/CE3 in CWR22Pc cells can induce a CRPCa phenotype and thus may negate the enrichment of CRPCa cells with a break fusion junction signature. Similarly, lentiviral expression of shRNA targeted to AR Exon 2b and/or Exon CE3 may prevent or delay the emergence of cells with a CRPCa phenotype and a break fusion junction signature. Where isogenic PCa xenograft pairs that progress to CRPCa through a route of AR gene alteration are identified, and one is able to establish PCR-based methods for monitoring the emergence of these alterations longitudinally, then one can design similar sets of characterization studies on these xenografts in vivo to expand the potential utility of these popular progression models.

MDV3100 is a next-generation antiandrogen that was designed to more effectively inhibit AR activity, even under conditions of AR overexpression (Tran et al., 2009 Science 324(5928):787-90). Data from a Phase II trial indicates that one-half of CRPCa patients receiving MDV3100 displayed a robust decrease in serum PSA levels (defined as a decrease of at least 50%), stabilized bone disease, and conversion of CTC counts from unfavorable to favorable (Scher et al., 2010 Lancet 375(9724):1437-46). These data indicate that mechanisms of resistance to MDV3100 pre-exist in nearly half of CRPCa patients. Moreover, in initial responders, resistance can develop during treatment (Scher et al., 2010 Lancet 375(9724):1437-46).

MDV3100 binds the AR LBD, and truncated AR isoforms may undermine the efficacy of this promising new therapeutic. However, a recent study has shown that MDV3100 can inhibit the androgen-independent growth of LNCaP cells expressing truncated AR isoforms, although the mechanism for this observation is unknown (Watson et al., 2010 Proc Natl Acad Sci USA, 107:16759-65). The CWR22Pc model of PCa progression can be used to test the effects of MDV3100 on the function of endogenous truncated AR isoforms. One can therefore culture CWR22Pc cells in the absence of androgens for 30 days as outlined above, in conjunction with 10 μM MDV3100 or vehicle control (Watson et al., 2010 Proc Natl Acad Sci USA, 107:16759-65). Cells can be subjected to the same molecular and cellular assays outlined above. For this set of studies, in vivo xenograft experiments may be performed at, for example, only the Day 25 or Day 30 time point to establish whether MDV3100 treatment has prevented emergence of a CRPCa phenotype. Parallel experiments with 22Rv1 cells can be performed for the Day 0, Day 5, and Day 10 time points. These studies can be corroborated with shRNA-mediated inhibition with AR Exon 7-targeted shRNA (to model durable inhibition of the AR LBD) or AR Exon 1-targeted shRNA (to model durable inhibition of the AR NTD) (FIG. 32). These shRNA-based evaluations can be carried out as outlined above for AR Exon 2b- or CE3-targeted shRNA.

Student's T-tests can be used to evaluate quantitative differences in all molecular and cellular parameters between experimental and control groups. MDV3100 may not impair androgen-independent AR activity or growth of 22Rv1 cells under castrate conditions in vitro or in vivo. Similarly, infection with AR Exon 7-targeted shRNA may not impair androgen-independent AR activity or growth of 22Rv1 cells under castrate conditions in vitro or in vivo. Similarly, neither MDV3100 nor AR Exon 7-targeted shRNA may block emergence of CRPCa cells harboring the 22Rv1 breakpoint signature during long-term CWR22Pc culture. Conversely, AR Exon 1-targeted shRNA may durably suppress 22Rv1 cell growth and/or prevent or delay emergence of CRPCa in the CWR22Pc progression model. Where truncated AR isoform activity and CRPCa growth are impervious to MDV3100 or full-length AR knock-down, one can use lentivirus-expressed shRNAs targeted to AR Exons 2b and/or CE3 to confirm that activity of these truncated AR isoforms contributes to resistance in emergent cells.

For any method disclosed herein that includes discrete steps, the steps may be conducted in any feasible order. And, as appropriate, any combination of two or more steps may be conducted simultaneously.

### Examples

### Example 1

### Steps

1. PCR AR or AR isoform from CMV5 vector (EcoRV FWD/Sal1 RV)
2. Cut PCR product with EcoRV/SalI, cut pLV with EcoRV/SalI
3. Ligate, screen.

### Step 1 PCR Primers:

Primer 1:
(SEQ ID NO: 20)
5′-TGGGATATCCAGCCAAGCTCAAGG-3′
Primer 2:
(SEQ ID NO: 21)
5′-GGGAGTCGACACAGGGATGCCA-3′

The “CMV5 vector” referred to in Step 1 is the expression construct that we use for all of our AR cDNAs. The two primers listed above can amplify any AR species from the CMV5 vector.

Plasmid 1/2/2b was generated by mutating h5HBhAR (this is shRNA-resistant wild-type AR in the CMV5 vector, it is described in Dehm et al., (2007 Cancer Res 67:10067-77) to generate an XbaI site within Exon 2 using mutagenic primers:

FW:
(SEQ ID NO: 22)
5′-GGAAGCTGCAAGGTCTTCTAGAAAAGAGCCGCTGAAGG-3′,
RV:
(SEQ ID NO: 23)
5′-CCTTCAGCGGCTCTTTTCTAGAAGACCTTGCAGCTTCC-3′,

and a Site Directed Mutagenesis Kit (Stratagene, Agilent Technologies, La Jolla, Calif.). Two oligonucleotides (SEQ ID NO:24 and SEQ ID NO:25) were synthesized and annealed to generate a cassette which contained Exon 2 sequence downstream from the XbaI site spliced to Exon 2b.

FW:
(SEQ ID NO: 24)
5′-CTAGAAAAGAGCCGCTGAAGGATTTTTCAGAATGAACAAATTAAAA
GAATCATAAG-3′
RV:
(SEQ ID NO: 25)
5′-CTAGCTTATGATTCTTTTAATTTGTTCATTCTGAAAAATCCTTCA
GCGGCTCTTTT-3′

This cassette was phosphorylated and inserted into XbaI-cut h5HBhAR. The XbaI site within Exon 2 was then converted back to wild-type sequence via site directed mutagenesis. The same strategy was used to generate 1/2/3/2b, 1/2/3/CE1, 1/2/3/CE2, and 1/2/3/CE3, but in this case the mutagenic primers used were:

FW:
(SEQ ID NO: 26)
5′-GAAGCAGGGATGACTCTAGAAGCCCGGAAGCTGAAG-3′
RV:
(SEQ ID NO: 27)
5′-CTTCAGCTTCCGGGCTTCTAGAGTCATCCCTGCTTC-3′
1/2/3/2b:
FW:
(SEQ ID NO: 28)
5′-CTAGGAGGATTTTTCAGAATGAACAAATTAAAAGAATCATAAT-3′
RV:
(SEQ ID NO: 29)
5′-CTAGATTATGATTCTTTTAATTTGTTCATTCTGAAAAATCCTC-3′
1/2/3/CE1:
FW:
(SEQ ID NO: 30)
5′-ctagGAGCTGTTGTTGTTTCTGAAAGAATCTTGAGGGTGTTTGGAG
TCTCAGAATGGCTTCCTTAAt-3′
RV:
(SEQ ID NO: 31)
5′-ctagaTTAAGGAAGCCATTCTGAGACTCCAAACACCCTCAAGATTC
TTTCAGAAACAACAACAGCTC-3′
1/2/3/CE2
FW:
(SEQ ID NO: 32)
5′-ctagGAGCAGGCAGCAGAGTGTCATAAt-3′
RV:
(SEQ ID NO: 33)
5′-ctagaTTATGACACTCTGCTGCCTGCTC-3′
1/2/3/CE3:
FW:
(SEQ ID NO: 34)
5′-ctagGAGAAAAATTCCGGGTTGGCAATTGCAAGCATCTCAAAATGA
CCAGACCCTGAt-3′
RV:
(SEQ ID NO: 35)
5′-ctagaTCAGGGTCTGGTCATTTTGAGATGCTTGCAATTGCCAACCC
GGAATTTTTCTC-3′

The shRNA-resistant, wild-type androgen receptor in the CMV5 vector is the template upon which all other cDNAs were built. The cDNA sequence for this “parental vector” is shown in SEQ ID NO:19.

### Example 2

### Materials and Methods

Cell Culture.

Benign prostate BPH-1 cells were generously provided by Dr. Haojie Huang (University of Minnesota) and cultured in RPMI 1640 (Invitrogen; Carlsbad, Calif.) with 10% FBS (Invitrogen; Carlsbad, Calif.). The CRPCa 22Rv1 cell line was obtained from ATCC and cultured in RPMI 1640 medium with 10% FBS. Androgen-dependent PCa CWR22Pc cells were generously provided by Dr. Marja Nevalainen (Thomas Jefferson University; Dagvadorj et al., 2008 Clin Cancer Res 14:6062-72) and cultured in RPMI 1640 supplemented with 10% FBS, 2.5 mM L-glutamine, and 0.8 nM dihydrotestosterone (Sigma; St. Louis, Mo.). Cell growth in RMPI 1640 medium containing 10% charcoal-stripped serum (CSS)+/−1 nM DHT was monitored by crystal violet staining. For androgen response experiments, cells were cultured in RPMI 1640+10% CSS for 48 hours, treated at t=0 with 1 nM DHT (Sigma; St. Louis, Mo.) or vehicle (EtOH), and then harvested at indicated time points. For long-term androgen deprivation, CWR22Pc cells were cultured in RPMI 1640+10% CSS for seven days, and then split to fresh plates in RPMI 1640+10% CSS. Cells were trypsinized and re-seeded in RPMI 1640+10% CSS after an additional 10 days to disperse emerging foci of growth. Samples were harvested following 7, 12, 17, 22, 27, and 32 days of culture in RPMI 1640+10% CSS.

Western Blot.

Western blotting of CWR22Pc and 22Rv1 lysates with AR (Santa Cruz N-20), ERK-2 (Santa Cruz D-2), and ARV-7 (Precision Antibody #AG10008) antibodies was performed exactly as described (Dehm et al., 2008 Cancer Res 68:5469-77).

Quantitative Real-Time RT-PCR.

Total cellular RNA was isolated from CWR22Pc and 22Rv1 cells as described (Dehm and Tindall, 2006 J Biol Chem 281:27882-93). RNA was reverse transcribed using a RT kit and an oligo(dT) primer (Roche; Madison, Wis.). Absolute quantitation of AR mRNA species was performed using forward and reverse primers listed in Table 4.

TABLE 4
Primer Sequences

| PRIMER NAME | USE | SEQUENCE 5′------->3′ | SEQ ID NO: |
|---|---|---|---|
| qSPLICE Exon3 FW | qRT-PCR | AAC AGA AGT ACC TGT GCG CC | 47 |
| qSPLICE CE1 RV | qRT-PCR | TGA GAC TCC AAA CAC CCT CA | 48 |
| qSPLICE CE2 RV | qRT-PCR | TAT GAC ACT CTG CTG CCT GC | 49 |
| qSPLICE CE3 RV | qRT-PCR | TCA GGG TCT GGT CAT TTT GA | 50 |
| qSPLICE Exon2b RV | qRT-PCR | TTC TGT CAG TCC CAT TGG TG | 51 |
| qSPLICE Exon2/2b FW | qRT-PCR | TGG ATG GAT AGC TAC TCC GG | 52 |
| qSPLICE Exon2/2b RV | qRT-PCR | GTT CAT TCT GAA AAA TCC TTC AGC | 53 |
| qSPLICE Exon1 FW | qRT-PCR | TGG ATG GAT AGC TAC TCC GG | 54 |
| qSPLICE Exon2 RV | qRT-PCR | CCC AGA AGC TTC ATC TCC AC | 55 |
| qSPLICE Exon4 RV | qRT-PCR | TTC AGA TTA CCA AGT TTC TTC AGC | 56 |
| qGENOME A FW | Genomic qPCR | TGG ATG GAT AGC TAC TCC GG | 57 |
| qGENOME A RV | Genomic qPCR | TTT ACC CTG CTG AGC TCT CC | 58 |
| qGENOME B FW | Genomic qPCR | GTG GAA GCT GCA AGG TCT TC | 59 |
| qGENOME B RV | Genomic qPCR | TAT TTG ATA GGG CCT TGC CA | 60 |
| qGENOME C FW | Genomic qPCR | CCT CCT CTG TTC CAA ACA GG | 61 |
| qGENOME C RV | Genomic qPCR | TTC TGT CAG TCC CAT TGG TG | 62 |
| qGENOME D FW | Genomic qPCR | TGT CCA TCT TGT CGT CTT CG | 63 |
| qGENOME D RV | Genomic qPCR | TGT GTC TAG AGC ATG GCT GG | 64 |
| qGENOME E FW | Genomic qPCR | GCA ATT GCA AGC ATC TCA AA | 65 |
| qGENOME E RV | Genomic qPCR | CAA CCC CAA CGT CAA AGT CT | 66 |
| qGENOME F FW | Genomic qPCR | CAC CTC CTT GTC AAC CCT GT | 67 |
| qGENOME F RV | Genomic qPCR | GAA AGG ATC TTG GGC ACT TG | 68 |
| qGENOME G FW | Genomic qPCR | CGG CAG ATA CAA TGT GAT GG | 69 |
| qGENOME G RV | Genomic qPCR | TTC CAA ACC TGC TGA TAG GG | 70 |
| qGENOME H FW | Genomic qPCR | CTA CCC AGA TCT TTT GCC CA | 71 |
| qGENOME H RV | Genomic qPCR | CAA CCC ACA GAA TGA GAG CA | 72 |
| qGENOME I FW | Genomic qPCR | GGC CTT GTA GAA TGA GTT TGG | 73 |
| qGENOME I RV | Genomic qPCR | TGA AAA GTC TCC CCA GCA GT | 74 |
| qGENOME J FW | Genomic qPCR | TTG CTT GGA GAG TTT CGT CC | 75 |
| qGENOME J RV | Genomic qPCR | GGG AGA CAG GAA AGA AGG AA | 76 |
| qGENOME K FW | Genomic qPCR | CCA TTG GGA CTG TGC TAG GT | 77 |
| qGENOME K RV | Genomic qPCR | CTG GCT TCA CCA CTG ACT GA | 78 |
| qGENOME L FW | Genomic qPCR | TCT CCC CTC TCC TGT CTT CA | 79 |
| qGENOME L RV | Genomic qPCR | GGG GAA CAT GCG ACC TAG TA | 80 |
| qGENOME M FW | Genomic qPCR | AGG GGT TCA TGT GCA TGT TT | 81 |
| qGENOME M RV | Genomic qPCR | TTG AGG TTG TAG GGT GGG AG | 82 |
| qGENOME N FW | Genomic qPCR | CAG TGA GCC CCT TTC TTC TG | 83 |
| qGENOME N RV | Genomic qPCR | GGC CAG GCA AAA GAA TAT CA | 84 |
| qGENOME O FW | Genomic qPCR | GGT TTG GAG GAG GAA GGA AG | 85 |
| qGENOME O RV | Genomic qPCR | GCA GAT CCC CAA CAG TCC TA | 86 |
| qGENOME P FW | Genomic qPCR | TCC TGC TTT CTG CCA TTC TT | 87 |
| qGENOME P RV | Genomic qPCR | CTT GGG ACA CAG TGA ACC CT | 88 |
| qGENOME Q FW | Genomic qPCR | TAA ATG GTC TGG CAA CTC CC | 89 |
| qGENOME Q RV | Genomic qPCR | CTT GGA CAC AGC TCC ACA GA | 90 |
| qGENOME R FW | Genomic qPCR | TGG GTG ACA GAG GAA GAT CC | 91 |
| qGENOME R RV | Genomic qPCR | TGC TCT CAT CTG TGT CTG GC | 92 |
| qGENOME S FW | Genomic qPCR | CTG TGA CCA GGG AGA ATG GT | 93 |
| qGENOME S RV | Genomic qPCR | TTC AGA TTA CCA AGT TTC TTC AGC | 94 |
| Breakpoint T FW | Outward-facing PCR | TTG AGG ACT TCA GCC TTT CAC CGC | 95 |
| Breakpoint T RV | Outward-facing PCR | GTG CAG CAG TCA CCT GAG AA | 96 |
| Breakpoint U FW | Outward-facing PCR | GGG TAC GTG TGC ACA ACT TG | 97 |
| Genome V FW | Genomic PCR control | AGC TGC AGG TCT GTT GGA GT | 98 |
| Genome V RV | Genomic PCR control | GCC TTG AAG GTC CTT TCC AT | 99 |

Concurrently, quantitative PCR with serial dilutions of plasmids harboring wild-type AR, AR 1/2/2b, AR 1/2/3/2b, AR 1/2/3/CE1, AR 1/2/3/CE2, and AR 1/2/3/CE3 cDNAs was performed using a SYBRGreen fastmix (PerfeCTa, VWR Life Sciences; Radnor, Pa.) and an iCycler instrument (BioRad; Hercules, Calif.) exactly as described (Dehm et al., 2008 Cancer Res 68:5469-77). Threshold cycle of amplification (Ct) values obtained from cDNA standards were used to construct Ct vs. cDNA standard copy number standard curves. Ct values obtained from real-time RT-PCR were plotted on these standard curves to derive copy number values for individual AR mRNA isoforms. For relative quantitation, fold expression change relative to GAPDH was determined by the comparative Ct method (2^{-ΔΔCt}).

Genomic PCR.

Genomic DNA was isolated from BPH-1, CWR22Pc, and 22Rv1 cells using a Nucleospin Kit (Clontech; Mountain View, Calif.). Genomic DNA from clinical CRPCa tissues was isolated as described previously (Liu et al., 2009 Nat Med 15:559-65). PCR primers designed using the Primer3 program of the MacVector software package and are listed in Table 4. For copy number determination, quantitative PCR with serial dilutions of BPH-1 genomic DNA was performed for each primer pair using SYBRGreen fastmix and an iCycler instrument. Ct values obtained from BPH-1 genomic DNA dilutions were used to construct Ct vs. genomic copy number standard curves, with the inference that one BPH-1 genome contains one copy of the X chromosome and therefore one copy of the target region. Ct values obtained from test genomic DNA in real-time PCR reactions were plotted on these standard curves to derive genomic copy numbers for each of the PCR target regions. For conventional PCR, genomic DNA was amplified using a Taq Polymerase PCR kit (Qiagen; Valencia, Calif.) according to the manufacturer's protocol. For long range PCR, genomic DNA was amplified using outward facing primers (Table 4) and a LongRange PCR kit (Qiagen; Valencia, Calif.). Cloned PCR products originating from the AR locus were completely sequenced to identify the 22Rv1 AR locus break fusion junction.

Affymetrix Genome-Wide Human SNP Array 6.0 Analysis.

Affymetrix SNP6.0 profiling of primary PCa (Mao et al., 2010 Cancer Res, 70:5207-12) and metastatic CRPCa (Liu et al., 2009 Nat Med 15:559-65) was performed in previous studies. Raw data in .CEL format was obtained from the Gene Expression Omnnibus website (accession numbers GSE18333 and GSE14996). Copy numbers were calculated for each probeset using Partek Genomics Suite 6.4 analysis software with default settings. Briefly, for each probeset, raw intensity was corrected for fragment length and sequence, and the geometric means of allele intensity values were scaled to 1 (0 in Log 2 space). Copy number was calculated from these summarized intensities by normalizing intensity of each individual tumor samples to the mean intensity of the pooled noncancerous samples. Probe level copy number data was used as input in an algorithm designed to determine the collection of breakpoints that satisfy the maximum likelihood between the input data and the noise-free version. The detailed algorithm is described in the Supplemental Methods section below and is available in MATLAB (The MathWorks) upon request.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1
CAAGGGAGGUUACACCAAAUU
SEQ ID NO: 2
UUUGGUGUAACCUCCCUUGUU
SEQ ID NO: 3
GGAACUCGAUCGUAUCAUUUU
SEQ ID NO: 4
AAUGAUACGAUCGAGUUCCUU
SEQ ID NO: 5
gatccccGCCATACTGCATGGCAGCAttcaagagaTGCTGCCATGCAGTATGGCtttttggaaa
SEQ ID NO: 6
agcttttccaaaaaGCCATACTGCATGGCAGCAtctcttgaaTGCTGCCATGCAGTATGGCggg
SEQ ID NO: 7
gatccccGGAACTCGATCGTATCATTttcaagagaAATGATACGATCGAGTTCCtttttggaaa
SEQ ID NO: 8
agcttttccaaaaaGGAACTCGATCGTATCATTtctcttgaaAATGATACGATCGAGTTCCggg
SEQ ID NO: 9
AAC AGA AGT ACC TGT GCG CC
SEQ ID NO: 10
TGA GAC TCC AAA CAC CCT CA
SEQ ID NO: 11
TAT GAC ACT CTG CTG CCT GC
SEQ ID NO: 12
TCA GGG TCT GGT CAT TTT GA
SEQ ID NO: 13
TTC TGT CAG TCC CAT TGG TG
SEQ ID NO: 14
TGG ATG GAT AGC TAC TCC GG
SEQ ID NO: 15
GTT CAT TCT GAA AAA TCC TTC AGC
SEQ ID NO: 16
TGG ATG GAT AGC TAC TCC GG
SEQ ID NO: 17
CCC AGA AGC TTC ATC TCC AC
SEQ ID NO: 18
TTC AGA TTA CCA AGT TTC TTC AGC
SEQ ID NO: 19
CCCATTCGCCATTCAGGCTGCGCAACTGTTGGGAAGGGCGATCGGTGCGGGCCTCTTCGCTATTACGCCAGCTGGCGAAAGGGGGATGTGCTGC
AAGGCGATTAAGTTGGGTAACGCCAGGGTTTTCCCAGTCACGACGTTGTAAAACGACGGCCAGTGCCAAGCTGATCTATACATTGAATCAATAT
TGGCAATTAGCCATATTAGTCATTGGTTATATAGCATAAATCAATATTGGCTATTGGCCATTGCATACGTTGTATCTATATCATAATATGTACA
TTTATATTGGCTCATGTCCAATATGACCGCCATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAACGACCCCCGCCCATTGACGTCAATAATGACG
TATGTTCCCATAGTAACGCCAATAGGGACTTTCCATTGACGTCAATGGGTGGAGTATTTACGGTAAACTGCCCACTTGGCAGTACATCAAGTGT
ATCATATGCCAAGTCCGCCCCCTATTGACGTCAATGACGGTAAATGGCCCGCCTGGCATTATGCCCAGTACATGACCTTACGGGACTTTCCTAC
TTGGCAGTACATCTACGTATTAGTCATCGCTATTACCATGGTGATGCGGTTTTGGCAGTACACCAATGGGCGTGGATAGCGGTTTGACTCACGG
GGATTTCCAAGTCTCCACCCCATTGACGTCAATGGGAGTTTGTTTTGGCACCAAAATCAACGGGACTTTCCAAAATGTCGTAATAACCCCGCCC
CGTTGACGCAAATGGGCGGTAGGCGTGTACGGTGGGAGGTCTATATAAGCAGAGCTCGTTTAGTGAACCGTCAGAATTCAGATCTTGTCCACCG
TGTGTCTTCTTCTGCACGAGACTTTGAGGCTGTCAGAGCGCTTTTTGCGTGGTTGCTCCCGCAAGTTTCCTTCTCTGGAGCTTCCCGCAGGTGG
GCAGCTAGCTGCAGCGACTACCGCATCATCACAGCCTGTTGAACTCTTCTGAGCAAGAGAAGGGGAGGCGGGGTAAGGGAAGTAGGTGGAAGAT
TCAGCCAAGCTCAAGGATGGAAGTGCAGTTAGGGCTGGGAAGGGTCTACCCTCGGCCGCCGTCCAAGACCTACCGAGGAGCTTTCCAGAATCTG
TTCCAGAGCGTGCGCGAAGTGATCCAGAACCCGGGCCCCAGGCACCCAGAGGCCGCGAGCGCAGCACCTCCCGGCGCCAGTTTGCTGCTGCTGC
AGCAGCAGCAGCAGCAGCAGCAGCAGCAGCAGCAGCAGCAGCAGCAGCAGCAGCAGCAGCAGCAGCAAGAGACTAGCCCCAGGCAGCAGCAGCA
GCAGCAGGGTGAGGATGGTTCTCCCCAAGCCCATCGTAGAGGCCCCACAGGCTACCTGGTCCTGGATGAGGAACAGCAACCTTCACAGCCGCAG
TCGGCCCTGGAGTGCCACCCCGAGAGAGGTTGCGTCCCAGAGCCTGGAGCCGCCGTGGCCGCCAGCAAGGGGCTGCCGCAGCAGCTGCCAGCAC
CTCCGGACGAGGATGACTCAGCTGCCCCATCCACGTTGTCCCTGCTGGGCCCCACTTTCCCCGGCTTAAGCAGCTGCTCCGCTGACCTTAAAGA
CATCCTGAGCGAGGCCAGCACCATGCAACTCCTTCAGCAACAGCAGCAGGAAGCAGTATCCGAAGGCAGCAGCAGCGGGAGAGCGAGGGAGGCC
TCGGGGGCTCCCACTTCCTCCAAGGACAATTACTTAGGGGGCACTTCGACCATTTCTGACAACGCCAAGGAGTTGTGTAAGGCAGTGTCGGTGT
CCATGGGCCTGGGTGTGGAGGCGTTGGAGCATCTGAGTCCAGGGGAACAGCTTCGGGGGGATTGCATGTACGCCCCACTTTTGGGAGTTCCACC
CGCTGTGCGTCCCACTCCTTGTGCCCCATTGGCCGAATGCAAAGGTTCTCTGCTAGACGACAGCGCAGGCAAGAGCACTGAAGATACTGCTGAG
TATTCCCCTTTCAAGGGAGGTTACACCAAAGGGCTAGAAGGCGAGAGCCTAGGCTGCTCTGGCAGCGCTGCAGCAGGGAGCTCCGGGACACTTG
AACTGCCGTCTACCCTGTCTCTCTACAAGTCCGGAGCACTGGACGAGGCAGCTGCGTACCAGAGTCGCGACTACTACAACTTTCCACTGGCTCT
GGCCGGACCGCCGCCCCCTCCGCCGCCTCCCCATCCCCACGCTCGCATCAAGCTGGAGAACCCGCTGGACTACGGCAGCGCCTGGGCGGCTGCG
GCGGCGCAGTGCCGCTATGGGGACCTGGCGAGCCTGCATGGCGCGGGTGCAGCGGGACCCGGTTCTGGGTCACCCTCAGCCGCCGCTTCCTCAT
CCTGGCACACTCTCTTCACAGCCGAAGAAGGCCAGTTGTATGGACCGTGTGGTGGTGGTGGGGGTGGTGGCGGCGGCGGCGGCGGCGGCGGCGG
CGGCGGCGGCGGCGGCGGCGGCGGCGAGGCGGGAGCTGTAGCCCCCTACGGCTACACTCGGCCCCCTCAGGGGCTGGCGGGCCAGGAAAGCGAC
TTCACCGCACCTGATGTGTGGTACCCTGGCGGCATGGTGAGCAGAGTGCCCTATCCCAGTCCCACTTGTGTCAAAAGCGAAATGGGCCCCTGGA
TGGATAGCTACTCCGGACCTTACGGGGACATGCGTTTGGAGACTGCCAGGGACCATGTTTTGCCCATTGACTATTACTTTCCACCCCAGAAGAC
CTGCCTGATCTGTGGAGATGAAGCTTCTGGGTGTCACTATGGAGCTCTCACATGTGGAAGCTGCAAGGTCTTCTTCAAAAGAGCCGCTGAAGGG
AAACAGAAGTACCTGTGCGCCAGCAGAAATGATTGCACTATTGATAAATTCCGAAGGAAAAATTGTCCATCTTGTCGTCTTCGGAAATGTTATG
AAGCAGGGATGACTCTGGGAGCCCGGAAGCTGAAGAAACTTGGTAATCTGAAACTACAGGAGGAAGGAGAGGCTTCCAGCACCACCAGCCCCAC
TGAGGAGACAACCCAGAAGCTGACAGTGTCACACATTGAAGGCTATGAATGTCAGCCCATCTTTCTGAATGTCCTGGAAGCCATTGAGCCAGGT
GTAGTGTGTGCTGGACACGACAACAACCAGCCCGACTCCTTTGCAGCCTTGCTCTCTAGCCTCAATGAACTGGGAGAGAGACAGCTTGTACACG
TGGTCAAGTGGGCCAAGGCCTTGCCTGGCTTCCGCAACTTACACGTGGACGACCAGATGGCTGTCATTCAGTACTCCTGGATGGGGCTCATGGT
GTTTGCCATGGGCTGGCGATCCTTCACCAATGTCAACTCCAGGATGCTCTACTTCGCCCCTGATCTGGTTTTCAATGAGTACCGCATGCACAAG
TCCCGGATGTACAGCCAGTGTGTCCGAATGAGGCACCTCTCTCAAGAGTTTGGATGGCTCCAAATCACCCCCCAGGAATTCCTGTGCATGAAAG
CACTGCTACTCTTCAGCATTATTCCAGTGGATGGGCTGAAAAATCAAAAATTCTTTGATGAACTTCGAATGAACTACATCAAGGAACTCGATCG
TATCATTGCATGCAAAAGAAAAAATCCCACATCCTGCTCAAGACGCTTCTACCAGCTCACCAAGCTCCTGGACTCCGTGCAGCCTATTGCGAGA
GAGCTGCATCAGTTCACTTTTGACCTGCTAATCAAGTCACACATGGTGAGCGTGGACTTTCCGGAAATGATGGCAGAGATCATCTCTGTGCAAG
TGCCCAAGATCCTTTCTGGGAAAGTCAAGCCCATCTATTTCCACACCCAGTGAAGCATTGGAAACCCTATTTCCCCACCCCAGCTCATGCCCCC
TTTCAGATGTCTTCTGCCTGTTATAACTCTGCACTACTCCTCTGCAGTGCCTTGGGGAATTTCCTCTATTGATGTACAGTCTGTCATGCGCGTA
TCGATAAAGCTTGCATGCCTGCAGGTCGACTCTAGAGGATCCCGGGTGGCATCCCTGTGACCCCTCCCCAGTGCCTCTCCTGGCCCTGGAAGTT
GCCACTCCAGTGCCCACCAGCCTTGTCCTAATAAAATTAAGTTGCATCATTTTGTCTGACTAGGTGTCCTTCTATAATATTATGGGGTGGAGGG
GGGTGGTATGGAGCAAGGGGCAAGTTGGGAAGACAACCTGTAGGGCCTGCGGGGTCTATTGGGAACCAAGCTGGAGTGCAGTGGCACAATCTTG
GCTCACTGCAATCTCCGCCTCCTGGGTTCAAGCGATTCTCCTGCCTCAGCCTCCCGAGTTGTTGGGATTCCAGGCATGCATGACCAGGCTCAGC
TAATTTTTGTTTTTTTGGTAGAGACGGGGTTTCACCATATTGGCCAGGCTGGTCTCCAACTCCTAATCTCAGGTGATCTACCCACCTTGGCCTC
CCAAATTGCTGGGATTACAGGCGTGAACCACTGCTCCCTTCCCTGTCCTTCTGATTTTAAAATAACTATACCAGCAGGAGGACGTCCAGACACA
GCATAGGCTACCTGCCATGGCCCAACCGGTGGGACATTTGAGTTGCTTGCTTGGCACTGTCCTCTCATGCGTTGGGTCCACTCAGTAGATGCCT
GTTGAATTGGGTACGCGGCCAGCTTCTGTGGAATGTGTGTCAGTTAGGGTGTGGAAAGTCCCCAGGCTCCCCAGCAGGCAGAAGTATGCAAAGC
ATGCATCTCAATTAGTCAGCAACCAGGTGTGGAAAAGTCCCCAGGCTCCCCAGCAGGCAGAAGTATGCAAAGCATGCATCTCAATTAGTCAGCA
ACCATAGTCCCGCCCCTAACTCCGCCCATCCCGCCCCTAACTCCGCCCAGTTCCGCCCATTCTCCGCCCCATGGCTGACTAATTTTTTTTATTT
ATGCAGAGGCCGAGGCCGCCTCGGCCTCTGAGCTATTCCAGAAGTAGTGAGGAGGCTTTTTTGGAGGCCTAGGCTTTTGCAAAAAGCTCCTCGA
GGAACTGAAAAACCAGAAAGTTAATTCCCTATAGTGAGTCGTATTAAATTCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGCGAGCGGTATCAGCTCACTCAAAGGCGGTAATACGGTTATCCACA
GAATCAGGGGATAACGCAGGAAAGAACATGTGAGCAAAAGGCCAGCAAAAGGCCAGGAACCGTAAAAGGCCGCGTTGCTGGCGTTTTTCCATAG
GCTCCGCCCCCCTGACGAGCATCACAAAAATCGACGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCCTTTCTCCCTTCGGGAAGCGTGGCGCTTTCTCAAT
GCTCACGCTGTAGGTATCTCAGTTCGGTGTAGGTCGTTCGCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGGACAGTATTTGGTATCTGCGCTCTGCTGAAGCCAGTTA
CCTTCGGAAAAAGAGTTGGTAGCTCTTGATCCGGCAAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGAACGAAAACTCACGTTAAGGGATTTTGGTCATGAGA
TTATCAAAAAGGATCTTCACCTAGATCCTTTTAAATTAAAAATGAAGTTTTAAATCAATCTAAAGTATATATGAGTAAACTTGGTCTGACAGTT
ACCAATGCTTAATCAGTGAGGCACCTATCTCAGCGATCTGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACGCTCACCGGCTCCAGATTTATCAGCAATAAACCAGCCAGCC
GGAAGGGCCGAGCGCAGAAGTGGTCCTGCAACTTTATCCGCCTCCATCCAGTCTATTAATTGTTGCCGGGAAGCTAGAGTAAGTAGTTCGCCAG
TTAATAGTTTGCGCAACGTTGTTGCCATTGCTACAGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCCTCCGATCGTTGTCAGAAGTAAGTTGGCCGCAGTG
TTATCACTCATGGTTATGGCAGCACTGCATAATTCTCTTACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCATTTATCAGGGTTATTGTCTCATGAGCGGATACATATTTGAATGTATTTAGAA
AAATAAACAAATAGGGGTTCCGCGCACATTTCCCCGAAAAGTGCCACCTGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTTCGCTTTCTTCCCTTCCTTTCTCGCCACGTTCGCCGGCTTTCCCC
GTCAAGCTCTAAATCGGGGCATCCCTTTAGGGTTCCGATTTAGTGCTTTACGGCACCTCGACCCCAAAAAACTTGATTAGGGTGATGGTTCACG
TAGTGGGCCATCGCCCTGATAGACGGTTTTTCGCCCTTTGACGTTGGAGTCCACGTTCTTTAATAGTGGACTCTTGTTCCAAACTGGAACAACA
CTCAACCCTATCTCGGTCTATTCTTTTGATTTATAAGGGATTTTGCCGATTTCGGCCTATTGGTTAAAAAATGAGCTGATTTAACAAAAATTTA
ACGCGAATTTTAACAAAATATTAACAAAATATTAACGTTTACAATTTC
SEQ ID NO: 20
TGGGATATCCAGCCAAGCTCAAGG
SEQ ID NO: 21
GGGAGTCGACACAGGGATGCCA
SEQ ID NO: 22
GGAAGCTGCAAGGTCTTCTAGAAAAGAGCCGCTGAAGG
SEQ ID NO: 23
CCTTCAGCGGCTCTTTTCTAGAAGACCTTGCAGCTTCC
SEQ ID NO: 24
CTAGAAAAGAGCCGCTGAAGGATTTTTCAGAATGAACAAATTAAAAGAATCATAAG
SEQ ID NO: 25
CTAGCTTATGATTCTTTTAATTTGTTCATTCTGAAAAATCCTTCAGCGGCTCTTTT
SEQ ID NO: 26
GAAGCAGGGATGACTCTAGAAGCCCGGAAGCTGAAG
SEQ ID NO: 27
CTTCAGCTTCCGGGCTTCTAGAGTCATCCCTGCTTC-3′
SEQ ID NO: 28
CTAGGAGGATTTTTCAGAATGAACAAATTAAAAGAATCATAAT
SEQ ID NO: 29
CTAGATTATGATTCTTTTAATTTGTTCATTCTGAAAAATCCTC
SEQ ID NO: 30
ctagGAGCTGTTGTTGTTTCTGAAAGAATCTTGAGGGTGTTTGGAGTCTCAGAATGGCTTCCTTAAt
SEQ ID NO: 31
ctagaTTAAGGAAGCCATTCTGAGACTCCAAACACCCTCAAGATTCTTTCAGAAACAACAACAGCTC
SEQ ID NO: 32
ctagGAGCAGGCAGCAGAGTGTCATAAt
SEQ ID NO: 33
ctagaTTATGACACTCTGCTGCCTGCTC
SEQ ID NO: 34
ctagGAGAAAAATTCCGGGTTGGCAATTGCAAGCATCTCAAAATGACCAGACCCTGAt
SEQ ID NO: 35
ctagaTCAGGGTCTGGTCATTTTGAGATGCTTGCAATTGCCAACCCGGAATTTTTCTC

1. A method for detecting unbalanced amplification of a polynucleotide that encodes an androgen receptor (AR) comprising:
receiving a biological sample obtained from a subject, the biological sample comprising cells expressing a plurality of non-wild-type androgen receptors, each non-wild-type androgen receptor comprising a copy number;
measuring the copy number of at least a first polynucleotide that encodes a non-wild-type androgen receptor and a second polynucleotide that encodes a non-wild-type androgen receptor, thereby producing an expression ratio; and
identifying the sample as exhibiting an expression ratio of no less than a predetermined expression ratio, thereby detecting unbalanced amplification of the androgen receptor.

2. The method of claim 1 wherein the first non-wild-type androgen receptor comprises AR Exon 3.

3. The method of claim 1 wherein the second non-wild-type androgen receptor comprises AR Exon 8.

4. The method of claim 1 further comprising identifying the subject as at risk for androgen depletion-independent prostate cancer.

5. The method of claim 1 wherein the subject has received treatment for prostate cancer.

6. The method of claim 1 further comprising either initiating or modifying treatment of the subject based on detecting unbalanced amplification of a polynucleotide that encodes an androgen receptor.

7. The method of claim 6 wherein initiating or modifying treatment comprises administering to the subject at least one pharmaceutical composition effective for treating androgen depletion-independent prostate cancer.

8. The method of claim 1 wherein the predetermined expression ratio is 1.5:1.

9. A method of analyzing a biological sample from a subject, the method comprising:
receiving the biological sample, the biological sample comprising cells expressing a plurality of androgen receptor isoforms;
measuring expression of at least one androgen receptor isoform; and
identifying the sample as exhibiting a predetermined pattern of androgen receptor isoform expression if at least one of the following is true:
wild-type isoform is no more than a predetermined percentage of total androgen receptor isoform expression,
isoform 1/2/3/2b is expressed as a greater percentage of total androgen receptor isoform expression than is observed in a normal control, or
isoform 1/2/3/CE3 is expressed as a greater percentage of total androgen receptor isoform expression than is observed in a normal control.

10. The method of claim 9 further comprising identifying the subject as at risk for androgen depletion-independent prostate cancer.

11. The method of claim 9 wherein the subject has received treatment for prostate cancer.

12. The method of claim 9, further comprising either initiating or modifying treatment of the subject based on detecting unbalanced amplification of a polynucleotide that encodes an androgen receptor.

13. The method of claim 12 wherein initiating or modifying treatment comprises administering to the subject at least one pharmaceutical composition effective for treating androgen depletion-independent prostate cancer.
